(19) United States
(12) Patent Application Publication
(10) Pub. No.: US 2021/0147566 A1
(43) Pub. Date: May 20, 2021

(54) ANTIBODIES AND USES THEREOF

(71) Applicant: Distributed Bio, Inc., South San Francisco, CA (US)

(72) Inventors: Jacob Glanville, San Francisco, CA (US); Chelsea Jones, South San Fancisco, CA (US); David Maurer, South San Francisco, CA (US); Sawsan Youssef, Menlo Park, CA (US)

(21) Appl. No.: 17/072,258

(22) Filed: Oct. 16, 2020

Related U.S. Application Data
(60) Provisional application No. 62/923,420, filed on Oct. 18, 2019.

(51) Int. Cl. C07K 16/28; A61P 25/28; A61P 25/16; A61P 35/00; A61P 21/00; A61P 25/08; A61P 25/00; A61P 9/10; A61P 31/12

(52) U.S. Cl. CPC C07K 16/2881; A61K 2039/505; A61P 25/16; A61P 35/00; A61P 21/00; A61P 25/08; A61P 25/00; A61P 9/10; A61P 31/12; C07K 2317/56; C07K 2317/565; C07K 2317/526; C07K 2319/00; C07K 2317/31; C07K 2317/622; C07K 2317/77; C07K 2317/92; A61P 25/28

(57) ABSTRACT

Binding agents that bind to a transferrin receptor are provided. This transferrin receptor binding can allow binding agents to cross the blood-brain barrier and into other tissues, such as the eye and synovium. These binding agents can be utilized for diagnostic or therapeutic purposes. The binding agents can also be modified to improve their activity, to bind to more than one target antigen, or a combination thereof.

### CROSS REFERENCE

This application claims the benefit of U.S. Application No. 62/923,420, filed on Oct. 18, 2019, which application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 14, 2020, is named 44561-715_201_SL.txt and is 63,856 bytes in size.

### BACKGROUND

Transferrin receptors (TfRs) are transmembrane glycoproteins expressed by all nucleated cells of the body. These receptors mediate cellular uptake of iron from the plasma glycoprotein transferrin via receptor-mediated endocytosis of ligand-occupied TfR into specialized endosomes. Acidification within the endosomes leads to iron release. TfRs are involved in the development of erythrocytes and the nervous system and can positively regulate T and B cell proliferation through iron uptake.

### SUMMARY

The present disclosure provides antibodies, and antigen-binding fragments thereof, that selectively bind to a transferrin receptor (TfR) and are able to deliver a therapeutic agent to an immune privileged site for treatment of a disease or disorder.

In one aspect, provided herein is an antibody, or antigen-binding fragment thereof, that selectively binds to a transferrin receptor, that comprises a heavy chain variable region (VH) and a light chain variable region (VL), wherein the VL comprises a CDR3 having an amino acid sequence that is at least about 80%, 85%, 90%, 91%, 92%, 93%, 94% 95%, 96%, 97%, 98%, or 99% identical to any one of SEQ ID NOS: 47-59 or an amino acid sequence of CQX₁X₂X₃X₄X₅PX₆TF (SEQ ID NO: 47), wherein X₁ comprises Q or K; X₂ comprises S, A, G, Y, H; X₃ comprises Y, N, F, K, G, or L; X₄ comprises K, S, or R; X₅ comprises T, F, Y, A, L, R, P, or S; and X₆ comprises Y, W, F, R, L, or I.

In another aspect, provided herein is an antibody, or antigen-binding fragment thereof, that selectively binds to a transferrin receptor, that comprises a heavy chain variable region (VH) and a light chain variable region (VL), wherein the VL comprises a CDR1 having an amino acid sequence that is at least about 80%, 85%, 90%, 91%, 92%, 93%, 94% 95%, 96%, 97%, 98%, or 99% identical to any one of SEQ ID NOS: 13-27 or an amino acid sequence of RASQTLYTNYLA (SEQ ID NO: 26); KSSRSVLRTSKNKNFLA (SEQ ID NO: 27); or X₁ASX₂X₃X₄X₅X₆X₇LX₈ (SEQ ID NO: 13), wherein X₁ comprises R or Q; X₂ comprises Q or R; X₃ comprises G, D, S or N; X₄ comprises I or V; X₅ comprises S, R, G, N or K; X₆ comprises R, K, S, G, or D; X₇ comprises N, W, Y, A, R, or K; and X₈ comprises A or N.

In another aspect, provided herein is an antibody, or antigen-binding fragment thereof, that selectively binds to a transferrin receptor, that comprises a heavy chain variable region (VH) and a light chain variable region (VL), wherein the VL comprises a CDR2 having an amino acid sequence that is at least about 80%, 85%, 90%, 91%, 92%, 93%, 94% 95%, 96%, 97%, 98%, or 99% identical to any one of SEQ ID NOS: 32-41 or an amino acid sequence of X₁X₂X₃X₄X₅X₆X₇ (SEQ ID NO: 32), wherein X₁ comprises G, A, K, W, or S; X₂ comprises A or T; X₃ comprises F or S, X₄ comprises T, R, S, or N; X₅ comprises R or L; X₆ comprises R, Q, A, or E; and X₇ comprises S, N, or T.

In another aspect, provided herein is an antibody, or antigen-binding fragment thereof, that selectively binds to a transferrin receptor (TfR), that comprises a heavy chain variable region (VH) and a light chain variable region (VL), wherein the VH comprises a complementarity determining region (CDR) 3 (CDR3) having an amino acid sequence that is encoded by a nucleic acid sequence of SEQ ID NO: 1, or a nucleic acid sequence that is at least about 80%, 85%, 90%, 91%, 92%, 93%, 94% 95%, 96%, 97%, 98%, or 99% identical to SEQ ID NO: 8.

In another aspect, provided herein is an antibody, or antigen-binding fragment thereof, that selectively binds to a transferrin receptor, that comprises a heavy chain variable region (VH) and a light chain variable region (VL), wherein the VH comprises a complementarity determining region CDR3 having an amino acid sequence that is at least about 80%, 85%, 90%, 91%, 92%, 93%, 94% 95%, 96%, 97%, 98%, or 99% identical to SEQ ID NO: 7.

In another aspect, provided herein is an antibody, or antigen-binding fragment thereof, that selectively binds to a transferrin receptor, that comprises a heavy chain variable region (VH) and a light chain variable region (VL), wherein the VL comprises: (i) a complementarity determining region (CDR) 1 (CDR1) having an amino acid sequence that is at least about 80%, 85%, 90%, 91%, 92%, 93%, 94% 95%, 96%, 97%, 98%, or 99% identical to any one of SEQ ID NOS: 13-27 or an amino acid sequence of RASQTLYTNYLA (SEQ ID NO: 26); KSSRSVLRTSKNKNFLA (SEQ ID NO: 27); or X₁ASX₂X₃X₄X₅X₆X₇LX₈ (SEQ ID NO: 13), wherein X₁ comprises R or Q; X₂ comprises Q or R; X₃ comprises G, D, S or N; X₄ comprises I or V; X₅ comprises S, R, G, N or K; X₆ comprises R, K, S, G, or D; X₇ comprises N, W, Y, A, R, or K; and X₈ comprises A or N; (ii) a CDR2 having an amino acid sequence that is at least about 80%, 85%, 90%, 91%, 92%, 93%, 94% 95%, 96%, 97%, 98%, or 99% identical to any one of SEQ ID NOS: 32-41 or an amino acid sequence of X₁X₂X₃X₄X₅ X₆X₇ (SEQ ID NO: 32), wherein X₁ comprises G, A, K, W, or S; X₂ comprises A or T; X₃ comprises F or S, X₄ comprises T, R, S, or N; X₅ comprises R or L; X₆ comprises R, Q, A, or E; and X₇ comprises S, N, or T; and (iii) a CDR3 having an amino acid sequence that is at least about 80%, 85%, 90%, 91%, 92%, 93%, 94% 95%, 96%, 97%, 98%, or 99% identical to any one of SEQ ID NOS: 47-59 or an amino acid sequence of CQX₁X₂X₃X₄X₅PX₆TF (SEQ ID NO: 47), wherein X₁ comprises Q or K; X₂ comprises S, A, G, Y, H; X₃ comprises Y, N, F, K, G, or L; X₄ comprises K, S, or R; X₅ comprises T, F, Y, A, L, R, P, or S; and X₆ comprises Y, W, F, R, L, or I.

In another aspect, provided herein is an antibody, or antigen-binding fragment thereof, that selectively binds to a transferrin receptor, that comprises a heavy chain variable region (VH) and a light chain variable region (VL), wherein the VL comprises: (i) a complementarity determining region (CDR) 1 (CDR1) having an amino acid sequence of any one of SEQ ID NOS: 13-27 or an amino acid sequence of RASQTLYTNYLA (SEQ ID NO: 26); KSSRSVLRTSKNKNFLA (SEQ ID NO: 27); or X₁ASX₂X₃X₄X₅X₆X₇LX₈ (SEQ ID NO: 13), wherein X₁ comprises R or Q; X₂ comprises Q or R; X₃ comprises G, D, S or N; X₄ comprises I or V; X₅ comprises S, R, G, N or K; X₆ comprises R, K, S, G, or D; X₇ comprises N, W, Y, A, R, or K; and X₈ comprises A or N; (ii) a CDR2 having an amino acid sequence of any one of SEQ ID NOS: 32-41 or an amino acid sequence of X₁X₂X₃X₄X₅ X₆X₇ (SEQ ID NO: 32), wherein X₁ comprises G, A, K, W, or S; X₂ comprises A or T; X₃ comprises F or S, X₄ comprises T, R, S, or N; X₅ comprises R or L; X₆ comprises R, Q, A, or E; and X₇ comprises S, N, or T; and (iii) a CDR3 having an amino acid sequence of any one of SEQ ID NOS: 47-59 or an amino acid sequence of CQX₁X₂X₃X₄X₅PX₆TF (SEQ ID NO: 47), wherein X₁ comprises Q or K; X₂ comprises S, A, G, Y, H; X₃ comprises Y, N, F, K, G, or L; X₄ comprises K, S, or R; X₅ comprises T, F, Y, A, L, R, P, or S; and X₆ comprises Y, W, F, R, L, or I.

In another aspect, provided herein is an antibody, or antigen-binding fragment thereof, that selectively binds to a transferrin receptor, that comprises a heavy chain variable region (VH) and a light chain variable region (VL), wherein the VL comprises: (i) a complementarity determining region (CDR) 1 (CDR1) having an amino acid sequence of any one of SEQ ID NOS: 13-27; (ii) a CDR2 having an amino acid sequence of any one of SEQ ID NOS: 32-41; and (iii) a CDR3 having an amino acid sequence of any one of SEQ ID NOS: 47-59.

In another aspect, provided herein is an antibody, or antigen-binding fragment thereof, that selectively binds to a transferrin receptor, that comprises a heavy chain variable region (VH) and a light chain variable region (VL); wherein the VL comprises: (a) a FR1 having an amino acid sequence that is at least about 80%, 85%, 90%, 91%, 92%, 93%, 94% 95%, 96%, 97%, 98%, or 99% identical to any one of SEQ ID NOS: 10-12 or amino acid sequence of X₁IX₂MTQSPX₃X₄LX₅X₆SX₇GX₈RX₉TX₁₀X₁₁C (SEQ ID NO: 9), wherein X₁ comprises D or E; X₂ comprises Q or V; X₃ comprises S, D, or A; X₄ comprises S or T; X₅ comprises S or A; X₆ comprises A or V; X₇ comprises L or P; X₈ comprises D or E; X₉ comprises V or A, X₁₀ comprises I or L; and X₁₁ comprises T, N, or S; (b) a FR2 having an amino acid sequence that is at least about 80%, 85%, 90%, 91%, 92%, 93%, 94% 95%, 96%, 97%, 98%, or 99% identical to any one of SEQ ID NOS: 29-31 or amino acid sequence of WYQQKPGXX₂PX₃LLIY (SEQ ID NO: 28), wherein X₁ comprises Q or K; X₂ comprises A or P; and X₃ comprises R or K; (c) a FR3 having an amino acid sequence that is at least about 80%, 85%, 90%, 91%, 92%, 93%, 94% 95%, 96%, 97%, 98%, or 99% identical to any one of SEQ ID NOS: 44-46 or amino acid sequence of GX₁PX₂RFSGSGSGTX₃FTLTISSLQX₄EDX₅AX₆YY (SEQ ID NO: 43), wherein X₁ comprises I or V; X₂ comprises A, D, or S; X₃ comprises E or D; X₄ comprises S. P, or A; X₅ comprises F or V; and X₆ comprises V or T; (d) a FR4 having an amino acid sequence that is at least about 80%, 85%, 90%, 91%, 92%, 93%, 94% 95%, 96%, 97%, 98%, or 99% identical to any one of SEQ ID NOS: 61-63 or amino acid sequence of GXGTX₂X₃X₄IK (SEQ ID NO: 60), wherein X₁ comprises G, Q, or P; X₂ comprises K or R; X₃ comprises L or V; and X₄ comprises E or D.

In another aspect, provided herein is an antibody, or antigen-binding fragment thereof, that selectively binds to a transferrin receptor, that comprises a heavy chain variable region (VH) and a light chain variable region (VL); wherein the VH comprises a FR1 having an amino acid sequence that is at least about 80%, 85%, 90%, 91%, 92%, 93%, 94% 95%, 96%, 97%, 98%, or 99% identical to SEQ ID NO: 1; a FR2 having an amino acid sequence that is at least about 80%, 85%, 90%, 91%, 92%, 93%, 94% 95%, 96%, 97%, 98%, or 99% identical to SEQ ID NO: 2; a FR3 having an amino acid sequence that is at least about 80%, 85%, 90%, 91%, 92%, 93%, 94% 95%, 96%, 97%, 98%, or 99% identical to SEQ ID NO: 3; and a FR4 having an amino acid sequence that is at least about 80%, 85%, 90%, 91%, 92%, 93%, 94% 95%, 96%, 97%, 98%, or 99% identical to SEQ ID NO: 4.

In another aspect, provided herein is an antibody, or antigen-binding fragment thereof, that selectively binds to a transferrin receptor, that comprises a heavy chain variable region (VH) and a light chain variable region (VL), wherein the VH comprises a CDR3 amino acid sequence that is encoded by SEQ ID NO: 8, or an amino acid sequence that is encoded by a nucleic acid sequence that is at least about 80%, 85%, 90%, 91%, 92%, 93%, 94% 95%, 96%, 97%, 98%, or 99% identical to SEQ ID NO: 8.

In another aspect, provided herein is an antibody, or antigen-binding fragment thereof, that selectively binds to a transferrin receptor, that comprises a heavy chain variable region (VH) and a light chain variable region (VL), wherein the VH comprises a CDR1 having an amino acid sequence that is at least about 80%, 85%, 90%, 91%, 92%, 93%, 94% 95%, 96%, 97%, 98%, or 99% identical to SEQ ID NO: 4; a CDR2 having an amino acid sequence that is at least about 80%, 85%, 90%, 91%, 92%, 93%, 94% 95%, 96%, 97%, 98%, or 99% identical to SEQ ID NO: 3; and a CDR3 having an amino acid sequence that is at least about 80%, 85%, 90%, 91%, 92%, 93%, 94% 95%, 96%, 97%, 98%, or 99% identical to SEQ ID NO: 6.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 26; (ii) a VL CDR2 having an amino acid sequence that is at least about 80%, 85%, 90%, 91%, 92%, 93%, 94% 95%, 96%, 97%, 98%, or 99% identical to SEQ ID NO: 33; and (iii) a VL CDR3 having an amino acid sequence that is at least about 80%, 85%, 90%, 91%, 92%, 93%, 94% 95%, 96%, 97%, 98%, or 99% identical to SEQ ID NO: 48.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 15; (ii) a VL CDR2 having an amino acid sequence that is at least about 80%, 85%, 90%, 91%, 92%, 93%, 94% 95%, 96%, 97%, 98%, or 99% identical to SEQ ID NO: 34; and (iii) a VL CDR3 having an amino acid sequence that is at least about 80%, 85%, 90%, 91%, 92%, 93%, 94% 95%, 96%, 97%, 98%, or 99% identical to SEQ ID NO: 49.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 14; (ii) a VL CDR2 having an amino acid sequence that is at least about 80%, 85%, 90%, 91%, 92%, 93%, 94% 95%, 96%, 97%, 98%, or 99% identical to SEQ ID NO: 35; and (iii) a VL CDR3 having an amino acid sequence that is at least about 80%, 85%, 90%, 91%, 92%, 93%, 94% 95%, 96%, 97%, 98%, or 99% identical to SEQ ID NO: 50.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 16; (ii) a VL CDR2 having an amino acid sequence that is at least about 80%, 85%, 90%, 91%, 92%, 93%, 94% 95%, 96%, 97%, 98%, or 99% identical to SEQ ID NO: 36; and (iii) a VL CDR3 having an amino acid sequence that is at least about 80%, 85%, 90%, 91%, 92%, 93%, 94% 95%, 96%, 97%, 98%, or 99% identical to SEQ ID NO: 51.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 17; (ii) a VL CDR2 having an amino acid sequence that is at least about 80%, 85%, 90%, 91%, 92%, 93%, 94% 95%, 96%, 97%, 98%, or 99% identical to SEQ ID NO: 35; and (iii) a VL CDR3 having an amino acid sequence that is at least about 80%, 85%, 90%, 91%, 92%, 93%, 94% 95%, 96%, 97%, 98%, or 99% identical to SEQ ID NO: 50.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 18; (ii) a VL CDR2 having an amino acid sequence that is at least about 80%, 85%, 90%, 91%, 92%, 93%, 94% 95%, 96%, 97%, 98%, or 99% identical to SEQ ID NO: 37; and (iii) a VL CDR3 having an amino acid sequence that is at least about 80%, 85%, 90%, 91%, 92%, 93%, 94% 95%, 96%, 97%, 98%, or 99% identical to SEQ ID NO: 50.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 19; (ii) a VL CDR2 having an amino acid sequence that is at least about 80%, 85%, 90%, 91%, 92%, 93%, 94% 95%, 96%, 97%, 98%, or 99% identical to SEQ ID NO: 33; and (iii) a VL CDR3 having an amino acid sequence that is at least about 80%, 85%, 90%, 91%, 92%, 93%, 94% 95%, 96%, 97%, 98%, or 99% identical to SEQ ID NO: 52.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 27; (ii) a VL CDR2 having an amino acid sequence that is at least about 80%, 85%, 90%, 91%, 92%, 93%, 94% 95%, 96%, 97%, 98%, or 99% identical to SEQ ID NO: 38; and (iii) a VL CDR3 having an amino acid sequence that is at least about 80%, 85%, 90%, 91%, 92%, 93%, 94% 95%, 96%, 97%, 98%, or 99% identical to SEQ ID NO: 53.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 20; (ii) a VL CDR2 having an amino acid sequence that is at least about 80%, 85%, 90%, 91%, 92%, 93%, 94% 95%, 96%, 97%, 98%, or 99% identical to SEQ ID NO: 39; and (iii) a VL CDR3 having an amino acid sequence that is at least about 80%, 85%, 90%, 91%, 92%, 93%, 94% 95%, 96%, 97%, 98%, or 99% identical to SEQ ID NO: 54.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 21; (ii) a VL CDR2 having an amino acid sequence that is at least about 80%, 85%, 90%, 91%, 92%, 93%, 94% 95%, 96%, 97%, 98%, or 99% identical to SEQ ID NO: 40; and (iii) a VL CDR3 having an amino acid sequence that is at least about 80%, 85%, 90%, 91%, 92%, 93%, 94% 95%, 96%, 97%, 98%, or 99% identical to SEQ ID NO: 55.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 22; (ii) a VL CDR2 having an amino acid sequence that is at least about 80%, 85%, 90%, 91%, 92%, 93%, 94% 95%, 96%, 97%, 98%, or 99% identical to SEQ ID NO: 41; and (iii) a VL CDR3 having an amino acid sequence that is at least about 80%, 85%, 90%, 91%, 92%, 93%, 94% 95%, 96%, 97%, 98%, or 99% identical to SEQ ID NO: 56.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 23; (ii) a VL CDR2 having an amino acid sequence that is at least about 80%, 85%, 90%, 91%, 92%, 93%, 94% 95%, 96%, 97%, 98%, or 99% identical to SEQ ID NO: 33; and (iii) a VL CDR3 having an amino acid sequence that is at least about 80%, 85%, 90%, 91%, 92%, 93%, 94% 95%, 96%, 97%, 98%, or 99% identical to SEQ ID NO: 57.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 24; (ii) a VL CDR2 having an amino acid sequence that is at least about 80%, 85%, 90%, 91%, 92%, 93%, 94% 95%, 96%, 97%, 98%, or 99% identical to SEQ ID NO: 42; and (iii) a VL CDR3 having an amino acid sequence that is at least about 80%, 85%, 90%, 91%, 92%, 93%, 94% 95%, 96%, 97%, 98%, or 99% identical to SEQ ID NO: 58.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a VL CDR1 having an amino acid sequence that is at least about 80%, 85%, 90%, 91%, 92%, 93%, 94% 95%, 96%, 97%, 98%, or 99% identical to SEQ ID NO: 25; (ii) a VL CDR2 having an amino acid sequence that is at least about 80%, 85%, 90%, 91%, 92%, 93%, 94% 95%, 96%, 97%, 98%, or 99% identical to SEQ ID NO: 33; and (iii) a VL CDR3 having an amino acid sequence that is at least about 80%, 85%, 90%, 91%, 92%, 93%, 94% 95%, 96%, 97%, 98%, or 99% identical to SEQ ID NO: 59.

Alternatively or additionally, an antibody, or antigen-binding fragment thereof, that selectively binds to a transferrin receptor, that comprises a heavy chain variable region (VH) and a light chain variable region (VL), wherein the VL comprises: (i) a FR1 having an amino acid sequence that is at least about 80%, 85%, 90%, 91%, 92%, 93%, 94% 95%, 96%, 97%, 98%, or 99% identical to any one of SEQ ID NOS: 10-12 or amino acid sequence of X₁IX₂MTQSPX₃X₄LX₅X₆SX₇GXsRX₉TX₁₀X₁₁C (SEQ ID NO: 9), wherein X₁ comprises D or E; X₂ comprises Q or V; X₃ comprises S, D, or A; X₄ comprises S or T; X₅ comprises S or A; X₆ comprises A or V; X₇ comprises L or P; X₈ comprises D or E; X₉ comprises V or A, X₁₀ comprises I or L; and X₁₁ comprises T, N, or S; (ii) a CDR1 having an amino acid sequence that is at least about 80%, 85%, 90%, 91%, 92%, 93%, 94% 95%, 96%, 97%, 98%, or 99% identical to any one of SEQ ID NOS: 13-27 or an amino acid sequence of RASQTLYTNYLA (SEQ ID NO: 26); KSSRSVLRTSKNKNFLA (SEQ ID NO: 27); or X₁ASX₂X₃X₄X₅X₆X₇LX₈ (SEQ ID NO: 13), wherein X₁ comprises R or Q; X₂ comprises Q or R; X₃ comprises G, D, S or N; X₄ comprises I or V; X₅ comprises S, R, G, N or K; X₆ comprises R, K, S, G, or D; X₇ comprises N, W, Y, A, R, or K; and X₅ comprises A or N; (iii) a FR2 having an amino acid sequence that is at least about 80%, 85%, 90%, 91%, 92%, 93%, 94% 95%, 96%, 97%, 98%, or 99% identical to any one of SEQ ID NOS: 29-31 or amino acid sequence of WYQQKPGXX₂PX₃LLIY (SEQ ID NO: 28), wherein X₁ comprises Q or K; X₂ comprises A or P; and X₃ comprises R or K; (iv) a CDR2 having an amino acid sequence that is at least about 80%, 85%, 90%, 91%, 92%, 93%, 94% 95%, 96%, 97%, 98%, or 99% identical to any one of SEQ ID NOS:32-41 or an amino acid sequence of X₁X₂X₃X₄X₅ X₆X₇ (SEQ ID NO: 32), wherein X₁ comprises G, A, K, W, or S; X₂ comprises A or T; X₃ comprises F or S, X₄ comprises T, R, S, or N; X₅ comprises R or L; X₆ comprises R, Q, A, or E; and X₇ comprises S, N, or T; (v) a FR3 having an amino acid sequence that is at least about 80%, 85%, 90%, 91%, 92%, 93%, 94% 95%, 96%, 97%, 98%, or 99% identical to any one of SEQ ID NOS: 44-46 or amino acid sequence of GX₁PX₂RFSGSGSGTX₃FTLTISSLQX₄EDX₅AX₆YY (SEQ ID NO: 43), wherein X₁ comprises I or V; X₂ comprises A, D, or S; X₃ comprises E or D; X₄ comprises S. P, or A; X₅ comprises F or V; and X₆ comprises V or T; (vi) a CDR3 having an amino acid sequence that is 47-59 or an amino acid sequence of CQX₁X₂X₃X₄X₅PX₆TF (SEQ ID NO: 47), wherein X₁ comprises Q or K; X₂ comprises S, A, G, Y, H; X₃ comprises Y, N, F, K, G, or L; X₄ comprises K, S, or R; X₅ comprises T, F, Y, A, L, R, P, or S; and X₆ comprises Y, W, F, R, L, or I; and (vii) a FR4 having an amino acid sequence that is at least about 80%, 85%, 90%, 91%, 92%, 93%, 94% 95%, 96%, 97%, 98%, or 99% identical to any one of SEQ ID NOS: 61-63 or amino acid sequence of GX₁GTX₂X₃X₄IK (SEQ ID NO: 60), wherein X₁ comprises G, Q, or P; X₂ comprises K or R; X₃ comprises L or V; and X₄ comprises E or D.

Alternatively or additionally, an antibody, or antigen-binding fragment thereof, that selectively binds to a transferrin receptor, that comprises a heavy chain variable region (VH) and a light chain variable region (VL), wherein the VH comprises an amino acid sequence that is at least about 80%, 85%, 90%, 91%, 92%, 93%, 94% 95%, 96%, 97%, 98%, or 99% identical to SEQ ID NO: 87.

Alternatively or additionally, an antibody, or antigen-binding fragment thereof, that selectively binds to a transferrin receptor, that comprises a heavy chain variable region (VH) and a light chain variable region (VL), wherein the VL comprises an amino acid sequence that is at least about 80%, 85%, 90%, 91%, 92%, 93%, 94% 95%, 96%, 97%, 98%, or 99% identical to any one of SEQ ID NOS: 64-65, 70-74, or 80-86.

An antibody can be, for example, a monoclonal antibody, a chimeric antibody, a human antibody, a bi-valent antibody, a multi-valent antibody, a maxibody, a humanized antibody, a deimmunized antibody, a humanized and deimmunized antibody, a mimetic thereof, a conjugate thereof, a fusion thereof, or a combination thereof. An antigen-binding fragment can be, for example, a Fab, a Fab′, a F(ab′)₂, a Fv, a scFv, a triabody, a tetrabody, a minibody, a bispecific F(ab′)₂, a trispecific F(ab′)₂, a diabody, a bispecific diabody, a single chain binding polypeptide, or a bispecific scFv. An antibody, or antigen-binding fragment thereof, provided herein may comprise one or more modifications. An antibody, or antigen-binding fragment thereof, provided herein may be isolated, recombinant, native, synthetic, purified, or a combination thereof. An antibody, or antigen-binding fragment thereof, provided herein may be an IgG, an IgA, an IgD, an IgE, or an IgM.

An antibody, or antigen-binding fragment thereof, provided herein may have a binding affinity to the TfR of from about 1 nM to about 5 μM. In one instance, the antibody, or antigen-binding fragment thereof, comprises a binding affinity to TfR of from about 1 nM to about 500 nM, from about 50 nM to about 400 nM, from about 100 nM to about 300 nM, from about 150 nM to about 250 nM, or from about 175 nM to about 225 nM.

In one aspect, a modified antibody provided herein may comprise a first polypeptide and a second polypeptide, each comprising a C_{H}3antibody constant domain, wherein the first and second polypeptides meet at an engineered interface within the C_{H}3 domain, and wherein the first polypeptide or the second polypeptide comprises a VH that selectively binds to a transferrin receptor and the VH comprises a CDR3 that is encoded by the nucleic acid sequence of SEQ ID NO: 8, or a nucleic acid sequence that is at least about 80%, 85%, 90%, 91%, 92%, 93%, 94% 95%, 96%, 97%, 98%, or 99% identical to SEQ ID NO: 8.

In one instance, the VH comprises a complementarity determining region CDR3 having an amino acid sequence that is at least about 80%, 85%, 90%, 91%, 92%, 93%, 94% 95%, 96%, 97%, 98%, or 99% identical to SEQ ID NO: 6. For example, a VH may comprise a framework 1 (FR1) having an amino acid sequence that is at least about 80%, 85%, 90%, 91%, 92%, 93%, 94% 95%, 96%, 97%, 98%, or 99% identical to SEQ ID NO: 1, a FR2 having an amino acid sequence that is at least about 80%, 85%, 90%, 91%, 92%, 93%, 94% 95%, 96%, 97%, 98%, or 99% identical to SEQ ID NO: 3, a FR3 having an amino acid sequence that is at least about 80%, 85%, 90%, 91%, 92%, 93%, 94% 95%, 96%, 97%, 98%, or 99% identical to SEQ ID NO: 5, and a FR4 having an amino acid sequence that is at least about 80%, 85%, 90%, 91%, 92%, 93%, 94% 95%, 96%, 97%, 98%, or 99% identical to SEQ ID NO: 7.

In another instance, the VH comprises an amino acid sequence that is at least about 80%, 85%, 90%, 91%, 92%, 93%, 94% 95%, 96%, 97%, 98%, or 99% identical to SEQ ID NO: 87. For example, a VH may comprise a CDR1 having an amino acid sequence that is at least about 80%, 85%, 90%, 91%, 92%, 93%, 94% 95%, 96%, 97%, 98%, or 99% identical to SEQ ID NO: 2; a CDR2 having an amino acid sequence that is at least about 80%, 85%, 90%, 91%, 92%, 93%, 94% 95%, 96%, 97%, 98%, or 99% identical to SEQ ID NO: 4; and a CDR3 having an amino acid sequence that is at least about 80%, 85%, 90%, 91%, 92%, 93%, 94% 95%, 96%, 97%, 98%, or 99% identical to SEQ ID NO: 6.

In another instance, the first polypeptide or the second polypeptide comprises a VL that selectively binds to a transferrin receptor. In one exemplary modified antibody, the VL comprises: (i) a complementarity determining region (CDR) 1 (CDR1) having an amino acid sequence that is at least about 80%, 85%, 90%, 91%, 92%, 93%, 94% 95%, 96%, 97%, 98%, or 99% identical to any one of SEQ ID NOS: 13-27 or an amino acid sequence of RASQTLYTNYLA (SEQ ID NO: 26); KSSRSVLRTSKNKNFLA (SEQ ID NO: 27); or X₁ASX₂X₃X₄X₅X₆X₇LX₈ (SEQ ID NO: 13), wherein X₁ comprises R or Q; X₂ comprises Q or R; X₃ comprises G, D, S or N; X₄ comprises I or V; X₅ comprises S, R, G, N or K; X₆ comprises R, K, S, G, or D; X₇ comprises N, W, Y, A, R, or K; and X₈ comprises A or N; (ii) a CDR2 having an amino acid sequence that is at least about 80%, 85%, 90%, 91%, 92%, 93%, 94% 95%, 96%, 97%, 98%, or 99% identical to any one of SEQ ID NOS: 32-41 or an amino acid sequence of X₁X₂X₃X₄X₅ X₆X₇ (SEQ ID NO: 32), wherein X₁ comprises G, A, K, W, or S; X₂ comprises A or T; X₃ comprises F or S, X₄ comprises T, R, S, or N; X₅ comprises R or L; X₆ comprises R, Q, A, or E; and X₇ comprises S, N, or T; and (iii) a CDR3 having an amino acid sequence that is at least about 80%, 85%, 90%, 91%, 92%, 93%, 94% 95%, 96%, 97%, 98%, or 99% identical to any one of SEQ ID NOS: 47-59 or an amino acid sequence of CQX₁X₂X₃X₄X₅PX₆TF (SEQ ID NO: 47), wherein X₁ comprises Q or K; X₂ comprises S, A, G, Y, H; X₃ comprises Y, N, F, K, G, or L; X₄ comprises K, S, or R; X₅ comprises T, F, Y, A, L, R, P, or S; and X₆ comprises Y, W, F, R, L, or I. In another exemplary modified antibody, the VL comprises a FR1 having an amino acid sequence that is at least about 80%, 85%, 90%, 91%, 92%, 93%, 94% 95%, 96%, 97%, 98%, or 99% identical to any one of SEQ ID NOS: 10-12, a FR2 having an amino acid sequence that is at least about 80%, 85%, 90%, 91%, 92%, 93%, 94% 95%, 96%, 97%, 98%, or 99% identical to any one of SEQ ID NOS: 29-31, a FR2 having an amino acid sequence that is at least about 80%, 85%, 90%, 91%, 92%, 93%, 94% 95%, 96%, 97%, 98%, or 99% identical to any one of SEQ ID NOS: 44-46, and a FR2 having an amino acid sequence that is at least about 80%, 85%, 90%, 91%, 92%, 93%, 94% 95%, 96%, 97%, 98%, or 99% identical to any one of SEQ ID NOS: 61-63. In another exemplary modified antibody, the VL having an amino acid sequence that is at least about 80%, 85%, 90%, 91%, 92%, 93%, 94% 95%, 96%, 97%, 98%, or 99% identical to any one of SEQ ID NOS: 64-65, 70-74, or 80-86.

In certain modified antibodies, a first polypeptide may comprise an engineered protuberance in the interface of the first polypeptide within its C_{H}3 domain created by replacing at least one contact residue of the first polypeptide within its C_{H}3 domain, and a second polypeptide may comprise an engineered cavity in the interface of the second polypeptide within its CH3 domain. The engineered protuberance in the interface of the first polypeptide may be positional in the engineered cavity of the second polypeptide so as to form a protuberance-into-cavity mutant pair. The engineered interface within the C_{H}3 domain may comprise at least two protuberance-into-cavity mutant pairs. The at least two protuberance-into-cavity mutant pairs may be created by creating at least one protuberance and at least one cavity on the first polypeptide and creating at least one cavity and at least one protuberance on the second polypeptide. The at least two protuberance-into-cavity mutant pairs may be created by creating more than one protuberance on the first polypeptide and creating more than one cavity on the second polypeptide.

A C_{H}3 domain can be, for example, from an immunoglobulin selected from the group consisting of an IgG, an IgA, an IgD, an IgE, and an IgM. In one instance, the immunoglobulin comprises an IgG. The IgG may be a subtype selected from the group consisting of an IgG1, an IgG2a, an IgG2b, an IgG3, and an IgG4. The IgG may be is a human IgG. In any of such instances, the modified antibody may comprise a bispecific modified antibody or antigen-binding fragment thereof, a trispecific modified antibody or antigen-binding fragment thereof, or a tetraspecific modified antibody or antigen-binding fragment thereof. The modified antibody may comprise an isolated modified antibody or antigen-binding fragment thereof or a purified modified antibody or antigen-binding fragment thereof. In some instances, the modified antibody may comprise a human modified antibody or antigen-binding fragment thereof. The modified antibody may comprise an isolated modified antibody or antigen-binding fragment thereof or a purified modified antibody or antigen-binding fragment thereof. Any of the antibodies, antigen-binding fragments thereof, modified antibodies, or modified antigen-binding fragments thereof may be capable of crossing the blood-brain barrier.

Provided herein is binding agent that comprises an antibody or an antigen-binding fragment thereof, or a modified antibody or antigen-binding fragment thereof, wherein the binding agent selectively binds to a TfR and one or more brain agents. The binding agent may, in some instances, further comprise a linker. A linker may comprise a linker sequence consisting of from about 3 to about 50 amino acids. The linker may form a link between one or more components of the binding agent. The binding agent may further comprise a fusion protein, wherein the fusion protein comprises another protein bound to the C-terminal side of the binding agent. The fusion protein may comprise a lysosomal enzyme. The lysosomal enzyme may comprise a human iduronate 2-sulfatase. The binding agent may be isolated, purified, recombinant, and/or synthetic. The binding agent is capable of crossing the blood-brain barrier. A binding agent as described herein may be used to contact the binding agent with a central nervous system (CNS) of a subject. The one or more brain antigens may be, for example, beta-secretase 1 (BACE1), Abeta, epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2(LRRK2), parkin, presenilin 1, presenilin 2, gamma secretase, death receptor 6 (DR6), amyloid precursor protein (APP), p75 neurotrophin receptor (p75NTR), caspase 6, TRK A, TRK B, TRK C, a synucleins, R synucleins, gamma synucleins, Tau, vascular endothelial growth factor (VEGF), neuropilin, a Semaphorin (e.g., Semaphorin 3A, Semaphorin 4A, or Semaphorin 6A), myelin basic protein (MBP), MOG, PLP, MAG, aquaporin 4, glutamate receptor, or a combination thereof.

Provided herein is one or more isolated nucleic acid sequences that encode(s) an antibody, or an antigen-binding fragment thereof, a modified antibody or antigen-binding fragment thereof, or a binding agent described herein. Also provided herein is one or more isolated vectors that comprises the one or more nucleic acid sequences. Also provided herein is a recombinant host cell that comprises the one or more isolated nucleic acid sequences or the one or more isolated vectors.

Provided herein is a pharmaceutical composition or a medicament that comprises the antibody, or the antigen-binding fragment thereof, the modified antibody or antigen-binding fragment thereof, or the binding agent as described herein, and a pharmaceutically acceptable excipient. The pharmaceutical composition may comprise a therapeutically effective amount of the antibody or antigen-binding fragment thereof, the modified antibody or antigen-binding fragment thereof, or the binding agent.

Provided herein is a container that comprises the antibody or antigen-binding fragment thereof, the modified antibody or antigen-binding fragment thereof, or the binding agent as described herein. The container may comprise, for example, a prefilled syringe, an intravenous bag, a bottle, an ampoule, a vial, and the like.

Provided herein is a kit that comprises the antibody or antigen-binding fragment thereof, the modified antibody or antigen-binding fragment thereof, or the binding agent as described herein. The kit may further comprise a label describing the use of the antibody, or the antigen-binding fragment thereof, the modified antibody or antigen-binding fragment thereof, or the binding agent for the treatment of a neurological disease, a central nervous system (CNS) disease, a cancer or metastasis thereof, a neuroendocrine disease, a metabolic disease, or a combination thereof in a subject.

Any of the antibodies, or antigen-binding fragments thereof, modified antibodies or antigen-binding fragments thereof, or the binding agents may be used in the treatment of a disease or disorder. Provided herein is a method of treating a neurological disease, a central nervous system (CNS) disease, a cancer or metastasis thereof, a neuroendocrine disease, a metabolic disease, or a combination thereof, in a subject in need thereof, comprising administering to the subject the antibody, or the antigen-binding fragment thereof, the modified antibody or antigen-binding fragment thereof, or the binding agent as described herein and above, whereby the neurological disease, the central nervous system (CNS) disease, the cancer or metastasis thereof, the neuroendocrine disease, the metabolic disease, or the combination thereof, is treated.

The subject may be administered one or more doses of the antibody, or the antigen-binding fragment thereof, the modified antibody or antigen-binding fragment thereof, or the binding agent. The subject may be administered a therapeutically effective amount of the antibody, or the antigen-binding fragment thereof, the modified antibody or antigen-binding fragment thereof, or the binding agent. Any route of administration may be utilized depending upon the disease or disorder to be treated, and the subject to the treated. For example, the antibody, or the antigen-binding fragment thereof, the modified antibody or antigen-binding fragment thereof, or the binding agent may be administered to the subject via injection, implant, or orally. Injection comprises, for example, an intravitreal injection, a subcutaneous injection, a parenteral injection, or an intravenous injection.

Treatment may resolve one or more symptoms of the disease or disorder in the subject. Treatment comprises partial of complete resolution of one or more symptoms of the disease or disorder in the subject. In some instances, treatment prolongs the life of the subject. In other instances, treatment prolongs progression-free survival (PFS) of the subject.

Provided herein is a use of an antibody, or an antigen-binding fragment thereof, a modified antibody or antigen-binding fragment thereof, or a binding agent as described herein, for the treatment of a neurological disease, a central nervous system (CNS) disease, a cancer or metastasis thereof, the neuroendocrine disease, the metabolic disease, or a combination thereof, in a subject in need thereof.

Use of an antibody, or an antigen-binding fragment thereof, a modified antibody or antigen-binding fragment thereof, or a binding agent as described herein, in the manufacture of a medicament for the treatment of a neurological disease, a central nervous system (CNS) disease, a cancer or metastasis thereof, the neuroendocrine disease, the metabolic disease, or a combination thereof, in a subject in need thereof.

In such of uses, the subject may be administered one or more doses of the antibody, or the antigen-binding fragment thereof, the modified antibody or antigen-binding fragment thereof, or the binding agent. The subject may be administered a therapeutically effective amount of the antibody, or the antigen-binding fragment thereof, the modified antibody or antigen-binding fragment thereof, or the binding agent. Any route of administration may be utilized depending upon the disease or disorder to be treated, and the subject to the treated. For example, the antibody, or the antigen-binding fragment thereof, the modified antibody or antigen-binding fragment thereof, or the binding agent may be administered to the subject via injection, implant, or orally. Injection comprises, for example, an intravitreal injection, a subcutaneous injection, a parenteral injection, or an intravenous injection.

In such of uses, the medicament is formulated to be administrable to the subject until one or more symptoms of the neurological disease, the central nervous system (CNS) disease, the cancer or metastasis thereof, the neuroendocrine disease, the metabolic disease, or the combination thereof, are resolved. Treatment comprises partial of complete resolution of one or more symptoms of the disease or disorder in the subject. In some instances, treatment prolongs the life of the subject. In other instances, treatment prolongs progression-free survival (PFS) of the subject.

Treatment comprises partial of complete resolution of one or more symptoms of the disease or disorder in the subject. In some instances, treatment prolongs the life of the subject. In other instances, treatment prolongs progression-free survival (PFS) of the subject. the medicament is formulated to be administrable to the subject until partial of complete resolution of one or more symptoms of the neurological disease, the central nervous system (CNS) disease, the cancer or metastasis thereof, the neuroendocrine disease, the metabolic disease, or the combination thereof, are partially or completely resolved.

Provided herein is a use of an antibody, or an antigen-binding fragment thereof, a modified antibody or antigen-binding fragment thereof, or a binding agent as described herein, that can cross a blood-brain-barrier of a subject. Provided herein is a use of an antibody, or an antigen-binding fragment thereof, a modified antibody or antigen-binding fragment thereof, or a binding agent as described herein, in the manufacture of a medicament that can cross a blood-brain-barrier of a subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

All sequences mentioned in this specification are hereby incorporated by reference.

### BRIEF DISCLOSURE OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the Antibody Discovery Work Flow Chart.

FIGS. 2A-B provide construct design for anti-TFR light chain bi-specific constructs. FIG. 2A illustrates the IgG1 antibody construct design for a bi-specific antibody with an anti-TFR invariant light chain (dashed lines) and proprietary SuperHuman 2.0 (SH2.0) heavy chain variants (no shading) for secondary specific binding. FIG. 2B illustrates the IgG1 antibody construct design for a tri-specific antibody with an anti-TFR invariant light chain (dashed lines) and proprietary SH2.0 heavy chain variants (unshaded and dense hatch) for secondary and tertiary specific binding held together by a ‘Knobs-into-holes’ Fc constant heavy chain 3 (CH3) region.

FIG. 3 provides data from the ELISA screening methods.

FIG. 4 provides data from the FACS screening methods of the scFv.

FIG. 5A-B provide construct Design for IgG1 conversion.

FIG. 6 provides data from the FACS screening methods of the IgG.

FIGS. 7A-D illustrate cell line quality control (QC) by FACS. FIG. 7A provides the results in Molt4 cells for cells only (left panel), mouse IgG1k Isotype control (middle panel), and mouse IgG1k anti-Hu TfR (right panel). FIG. 7B provides the results of Daudi cells for cells only (left panel), mouse IgG1k Isotype control (middle panel), and mouse IgG1k anti-Hu TfR (right panel).

FIG. 7C provides the results of CHOZN cells for cells only (left panel), mouse IgG1k Isotype control (middle panel), and mouse IgG1k anti-Hu TfR (right panel). FIG. 7D provides the results of HEK293 cells for cells only (left panel), mouse IgG1k Isotype control (middle panel), and mouse IgG1k anti-Hu TfR (right panel).

FIG. 8 provides data from a FACS screening method example of one of our internal TFR light chain IgG A07 on two positive cell lines expressing TFR1.

FIG. 9 provides data from the internalization screening methods.

### DETAILED DESCRIPTION

Unless otherwise specified in the present description, scientific terms used regarding the present invention have meanings that are generally understood by a person skilled in the art. In general, nomenclatures and techniques applied to the cell and tissue culture, molecular biology, immunology, microbiology, genetics, protein and nucleic acid chemistry, and hybridization, which are described in the present description, are well known in the present technical field, and thus, are commonly used.

The methods and techniques of the present invention are carried out in accordance with conventional methods that are well known in the present technical field, in such ways as described in a variety of general reference documents cited and discussed throughout the present description and more specific reference documents, unless otherwise specified.

### Overview

Described herein are antibodies and antigen-binding fragments thereof including variable light chains which can specifically bind to a transferrin receptor (TfR). Antibodies and antigen-binding fragments as described herein can comprise a light (L) chain and a heavy (H) chain, wherein the L chain comprises a variable light (VL) region which can specifically bind to a transferrin receptor (TfR). In some cases, the H chain can specifically bind to, or have affinity to, one or more molecules which are not TfR. In such cases, the antibodies or antigen-binding fragments can be bivalent or multivalent (e.g., trivalent, quatravalent, etc.).

In some instances, the TfR can be a human TfR. Such antibodies and antigen-binding fragments can be transported across a membrane or a barrier via receptor-mediated endocytosis mediated by TfR. In some embodiments, an antibody or antigen-binding fragment which can specifically bind to TfR can bind to TfR on the surface of a cell, and be internalized into the cell

Described herein are other binding agents, which can comprise an antibody, or an antigen-binding fragment thereof. In some cases, a binding agent can be a heteromultimer or a modified antibody. Such binding agents can specifically bind to transferrin and can additionally bind to one or more agents or molecules in the brain. In some cases, the one or more agents or molecules can be druggable targets, or can be homing targets.

Once inside a cell or across a membrane or barrier, such a bivalent or multivalent antibody or antigen-binding fragment that selectively binds to TfR can bind to another molecule to which it has affinity via a second binding domain. Affinity of an antibody or antigen-binding fragment to a molecule which is not TfR can be greater than the affinity of the antibody or antigen-binding fragment that selectively binds to the TfR, can be the about same as the affinity of the antibody or antigen-binding fragment that selectively binds to the TfR, or it can be lower than the affinity of the antibody or antigen-binding fragment that selectively binds to the TfR.

In some cases, a bivalent or multivalent antibody or antigen-binding fragment that selectively binds to TfR can provide a therapeutic effect once inside a cell or across a membrane or barrier. Such a therapeutic effect can be the result of the bivalent or multivalent nature of the antibody or antigen-binding fragment. For example, the antibody or antigen-binding fragment can be immunotherapeutic, radioimmunotherapeutic, or conjugated to another therapeutic agent. If the antibody or antigen-binding fragment is conjugated to another therapeutic agent, the other therapeutic agent can be delivered to a region at, or around, the molecule which is not TfR. The other therapeutic agent can be released upon binding of the antibody or antigen-binding fragment, or the other therapeutic agent can exert activity while conjugated to the antibody or antigen-binding fragment.

Such an antibody or antigen-binding fragment can be administered to a subject in need of treatment. In such cases, the antibody or antigen-binding fragment can provide a therapeutic effect, and/or can alter a state of health or disease of the subject.

In some cases, transferrin in a subject's circulation can compete with an antibody or antigen-binding fragment described herein. Such competition can have an effect on the transport of the antibody or antigen-binding fragment. In some cases, transferrin levels can vary between subjects or fluctuate, affecting the effective dose of antibody or antigen-binding fragment during treatment. In such cases, the effective dose of the antibody or other therapeutic agent can also vary between subjects or fluctuate. In such cases, the amount of antibody or antigen-binding fragment administered to the subject can be adjusted based on the amount of transferrin present in the circulation.

In addition to, or instead of, the blood-brain barrier, some antibodies or antigen-binding fragments described herein can cross one or more other blood-tissue barriers, which can include, for example, the blood-cerebrospinal fluid (CSF) barrier (choroid plexus), the blood-testis barrier (Sertoli cells), the placenta (maternofetal interface), the blood-retina barrier (retinal pigment epithelium), or the blood-thymus barrier (epithelial reticular cells). Such antibodies or antigen-binding fragments can provide a therapeutic effect or deliver another therapeutic agent to a tissue or tissues via one or more blood-tissue barriers they are able or configured to cross.

In some cases, an antibody or antigen-binding fragment may be able to cross more than one blood-tissue barrier. In certain embodiments, such antibodies and antigen binding fragments can provide a therapeutic effect or deliver another therapeutic agent to multiple tissues, or each tissue, they enter. In various embodiments, additional specificity may be preferred or required. In such cases, for example, the other non-TfR molecule that the antibody or antigen-binding fragment selectively binds to can be tissue-specific, such that although the antibody or antigen-binding fragment can enter multiple types of tissues, a provided therapeutic effect or another therapeutic agent may be delivered to only a subset of those tissues.

For example, the antibody or antigen-binding fragment can transport via TfR-mediated endocytosis across the blood-brain barrier and into the brain. In some cases, such transport can occur across a barrier that is different than the blood-brain barrier, either in addition to, or instead of, the blood-brain barrier.

To facilitate transport across a barrier, an antibody or antigen-binding fragment can specifically bind to a TfR. In some cases, the antibody or antigen-binding fragment can bind to only TfR. In some cases, the antibody or antigen-binding fragment can bind to TfR more strongly than it can bind to other proteins. In some cases, the antibody or antigen-binding fragment can bind to TfR at least about 10, 50, 100, 200, 300, 400, 500, or 1000 times more strongly than other proteins. In some cases, the antibody or antigen-binding fragment can bind to TfR more strongly than it can bind to other proteins on a cell surface. In some cases, the antibody or antigen-binding fragment can bind to TfR at least about 10, 50, 100, 200, 300, 400, 500, or 1000 times more strongly than other proteins on a cell surface. In some cases, the antibody or antigen-binding fragment can bind to TfR more strongly than it can bind to other proteins at a blood-tissue barrier. In some cases, the antibody or antigen-binding fragment can bind to TfR at least about 10, 50, 100, 200, 300, 400, 500, or 1000 times more strongly than other proteins at a blood-tissue barrier. In some cases, the antibody or antigen-binding fragment can bind to TfR more strongly than it can bind to other proteins at the blood-brain barrier. In some cases, the antibody or antigen-binding fragment can bind to TfR at least about 10, 50, 100, 200, 300, 400, 500, or 1000 times more strongly than other proteins at the blood-brain barrier.

In one aspect, a binding agent, an antibody, or an antigen-binding fragment described herein may be bivalent or multivalent. In some cases, an antibody or antigen-binding fragment can specifically bind to another protein in addition to TfR. In such cases, the antibody or antigen-binding fragment can specifically bind to a protein at a cell surface, at a blood-tissue barrier, at the blood-brain barrier, in an interstitial space, in cerebrospinal fluid, in cytoplasm, in a nucleus, or in or on an organelle.

An antibody or antigen-binding fragment can have specificity for a druggable target in the brain. In such cases, the antibody or antigen-binding fragment can have affinity for a druggable target in the brain. A druggable target can be a biological target than can be known to or predicted to bind to a drug. In some embodiments, the antibody or antigen-binding fragment can deliver another therapeutic agent to a druggable target in the brain. A druggable target in the brain can include, for example, an amyloid β plaque, an ion channel, a pain receptor, a neurological target, or a combination thereof.

In one aspect, a binding agent or an antibody or antigen-binding fragment described herein can be selected based upon one or more characteristics, or may be optimized in view of one or more desired characteristics such as, for example, binding affinity, reduced immunogenicity, etc. In some cases, an antibody or antigen-binding fragment described herein can be considered when selecting or optimizing the agent. If the specificity is too broad, the antibody or antigen-binding fragment can produce side effects, which can be undesirable. Thus, an antibody or antigen-binding fragment herein can be specific to TfR and few other proteins. In some cases, the affinity of the binding agent or the antibody or antigen-binding fragment can be considered when selecting or optimizing the agent. If the affinity is too low, the binding agent, antibody or antigen-binding fragment may not bind to TfR strongly enough to be transported, or at all. Conversely, if the affinity is too high, the binding agent, or the antibody or antigen-binding fragment may bind to TfR too strongly and not be released once it has been transported into a cell or across a membrane or a barrier. That is, an interaction between TfR and the antibody or antigen-binding fragment which is too strong may be problematic as dissociation may not occur or take too long to occur. While a binding agent, or the antibody or antigen-binding fragment described herein should be able to specifically bind to a TfR, the binding agent, or the antibody or antigen-binding fragment should be capable of dissociating from the TfR once it has been transported into a cell or across a membrane or a barrier.

Also described herein are methods of treating a subject in need thereof, with a binding agent, or an antibody or antigen-binding fragment described herein.

The term “about,” as used herein, generally refers to a range that is 2%, 5%, 10%, 15% greater than or less than (±) a stated numerical value within the context of the particular usage. For example, “about 10” would include a range from 8.5 to 11.5. As used herein, the terms “about” and “approximately,” when used to modify a numeric value or numeric range, indicate that deviations of up to about 0.2%, about 0.5%, about 1%, about 2%, about 5%, about 7.5%, or about 10% (or any integer between about 1% and 10%) above or below the value or range remain within the intended meaning of the recited value or range.

As used in this specification and the appended claims, the singular forms “a,” “an,” and “the” include plural references unless the context clearly dictates otherwise. Thus, for example, references to “a method” include one or more methods, and/or steps of the type described herein and/or which will become apparent to those persons skilled in the art upon reading this disclosure.

“Non-naturally occurring” when applied to polypeptides means a polypeptide or a portion thereof which, by virtue of its origin or manipulation: (i) is present in a host cell as the recombinant expression product of a portion of an expression vector; (ii) is linked to a protein or other chemical moiety other than that to which it is linked in nature; or (iii) does not occur in nature, for example, a protein that is chemically manipulated by appending or adding at least one hydrophobic moiety to the protein so that the protein is in a form not found in nature. By “isolated” it is further meant a protein can be: (i) synthesized chemically or (ii) expressed in a host cell and purified away from associated and contaminating proteins. The term generally means a polypeptide that has been separated from other proteins and nucleic acids with which it naturally occurs. Typically, the polypeptide is also separated from substances such as antibodies or gel matrices (polyacrylamide) which are used to purify it.

As used herein, “substantially pure,” “isolated,” or “purified” refers to material which is at least 50% pure (i.e., free from contaminants), at least 60% pure, at least 70% pure, at least 80% pure, at least 85% pure, at least 90% pure, at least 91% pure, at least 92% pure, at least 93% pure, at least 94% pure, at least 95% pure, at least 96% pure, at least 97% pure, at least 98% pure, or at least 99% pure.

The terms “polypeptide,” “oligopeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other suitable modifications. It is understood that, because the polypeptides of this disclosure are based upon an antibody, the polypeptides can occur as single chains or associated chains.

“Polynucleotide” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses, or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO, or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl, or araldyl. Not all linkages in a polynucleotide need be identical. The preceding description applies to all polynucleotides referred to herein, including RNA and DNA.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well-known Smith Waterman algorithm may also be used to determine identity.

Ranges of desired degrees of sequence identity are from about 80% to about 100% and integer values therebetween. In general, this disclosure encompasses sequences with about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%, sequence identity with any sequence provided herein.

“X” or “Xaa” as used in amino acid sequences herein is intended to indicate that any of the twenty standard amino acids may be placed at this position unless specifically noted otherwise.

### Transferrin Receptors

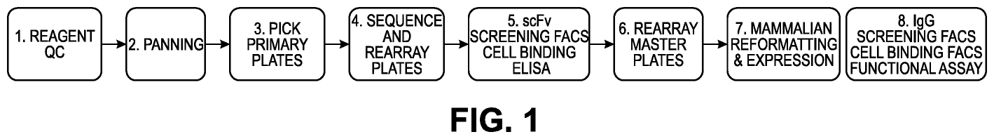

The human TfR cDNA encodes 760 amino acids (aa) including a 67 aa N-terminal intracellular domain, a 21 aa transmembrane domain, and a 672 aa extracellular domain (ECD) with helical, peptidase (nonfunctional), and ligand binding domains including an RGD potential integrin binding site. The TfR is a type 2 transmembrane glycoprotein expressed on erythroid progenitors, muscle cells, and proliferating cells as a 188 kDa disulfide-linked homodimer of 95 kDa monomers. Human TfR ECD shares 75-80% amino acid sequence identity with mouse, rat, feline, canine, equine, porcine, and bovine TfR. TfR can facilitate the transport of iron into cells, for example, via receptor mediated endocytosis (see, e.g., FIG. 1). In this process, an iron-transferrin complex can be identified by and bind to TfR on the surface of a cell, and the iron-transferrin complex can be internalized via endocytosis.

Human TfR is a single-pass transmembrane protein and is encoded by the TFRC gene on human chromosome 3. TfR can be a CD71 antigen, and it can be associated with incorporation of iron into cells and cell growth. The TfR of the present disclosure is not particularly limited in terms of structure. Thus, human TfR can include a monomer, a multimer, an intact form expressed on a cell membrane, a soluble form constituted in an extracellular region, a truncated form, a mutation form caused by genetic mutation, deletion, etc., and a form that has undergone posttranslational modification by phosphorylation or the like.

In one instance, the TfR comprises a Human TfR Protein 1 (UniProtKB-P02786; TFR1_HUMAN) having an amino acid sequence of: MMDQARSAFSNLFGGEPLSYTRFSLARQVDGDNSHVEMKLAVDEEENADNNTKANVTKP KRCSGSICYGTIAVIVFFLIGFMIGYLGYCKGVEPKTECERLAGTESPVREEPGEDFPAARRL YWDDLKRKLSEKLDSTDFTGTIKLLNENSYVPREAGSQKDENLALYVENQFREFKLSKVWR DQHFVKIQVKDSAQNSVIIVDKNGRLVYLVENPGGYVAYSKAATVTGKLVHANFGTKKDF EDLYTPVNGSIVIVRAGKITFAEKVANAESLNAIGVLIYMDQTKFPIVNAELSFFGHAHLGTG DPYTPGFPSFNHTQFPPSRSSGLPNIPVQTISRAAAEKLFGNMEGDCPSDWKTDSTCRMVTSE SKNVKLTVSNVLKEIKILNIFGVIKGFVEPDHYVVVGAQRDAWGPGAAKSGVGTALLLKLA QMFSDMVLKDGFQPSRSIIFASWSAGDFGSVGATEWLEGYLSSLHLKAFTYINLDKAVLGT SNFKVSASPLLYTLIEKTMQNVKHPVTGQFLYQDSNWASKVEKLTLDNAAFPFLAYSGIPA VSFCFCEDTDYPYLGTTMDTYKELIERIPELNKVARAAAEVAGQFVIKLTHDVELNLDYERY NSQLLSFVRDLNQYRADIKEMGLSLQWLYSARGDFFRATSRLTTDFGNAEKTDRFVMKKL NDRVMRVEYHFLSPYVSPKESPFRHVFWGSGSHTLPALLENLKLRKQNNGAFNETLFRNQL ALATWTIQGAANALSGDVWDIDNEF (SEQ ID NO: 110). In some instances, this amino acid sequence is further processed into an isoform.

In another instance, the TfR comprises a Human TfR Protein 2 (UniProtKB-Q9UP52; TFR2 HUMAN) isoform alpha (identifier: Q9UP52-1) having an amino acid sequence of:

(SEQ ID NO: 111)
MERLWGLFQRAQQLSPRSSQTVYQRVEGPRKGHLEEEEEDGEEGAETLAH
FCPMELRGPEPLGSRPRQPNLIPWAAAGRRAAPYLVLTALLIFTGAFLLG
YVAFRGSCQACGDSVLVVSEDVNYEPDLDFHQGRLYWSDLQAMFLQFLGE
GRLEDTIRQTSLRERVAGSAGMAALTQDIRAALSRQKLDHVWTDTHYVGL
QFPDPAHPNTLHWVDEAGKVGEQLPLEDPDVYCPYSAIGNVTGELVYAHY
GRPEDLQDLRARGVDPVGRLLLVRVGVISFAQKVTNAQDFGAQGVLIYPE
PADFSQDPPKPSLSSQQAVYGHVHLGTGDPYTPGFPSFNQTQFPPVASSG
LPSIPAQPISADIASRLLRKLKGPVAPQEWQGSLLGSPYHLGPGPRLRLV
VNNHRTSTPINNIFGCIEGRSEPDHYVVIGAQRDAWGPGAAKSAVGTAIL
LELVRTFSSMVSNGFRPRRSLLFISWDGGDFGSVGSTEWLEGYLSVLHLK
AVVYVSLDNAVLGDDKFHAKTSPLLTSLIESVLKQVDSPNHSGQTLYEQV
VFTNPSWDAEVIRPLPMDSSAYSFTAFVGVPAVEFSFMEDDQAYPFLHTK
EDTYENLHKVLQGRLPAVAQAVAQLAGQLLIRLSHDRLLPLDFGRYGDVV
LRHIGNLNEFSGDLKARGLTLQWVYSARGDYIRAAEKLRQEIYSSEERDE
RLTRMYNVRIMRVEFYFLSQYVSPADSPFRHIFMGRGDHTLGALLDHLRL
LRSNSSGRPGATSSTGFQESRFRRQLALLTWTLQGAANALSGDVWNIDNN
F.

In another instance, the TfR comprises a Human TfR Protein 2 (UniProtKB-Q9UP52; TFR2_HUMAN) isoform beta (identifier: Q9UP52-2) having an amino acid sequence of:

(SEQ ID NO: 114)
MAALTQDIRAALSRQKLDHVWTDTHYVGLQFPDPAHPNTLHWVDEAGKV
GEQLPLEDPDVYCPYSAIGNVTGELVYAHYGRPEDLQDLRARGVDPVGR
LLLVRVGVISFAQKVTNAQDFGAQGVLIYPEPADFSQDPPKPSLSSQQA
VYGHVHLGTGDPYTPGFPSFNQTQFPPVASSGLPSIPAQPISADIASRL
LRKLKGPVAPQEWQGSLLGSPYHLGPGPRLRLVVNNHRTSTPINNIFGC
IEGRSEPDHYVVIGAQRDAWGPGAAKSAVGTAILLELVRTFSSMVSNGF
RPRRSLLFISWDGGDFGSVGSTEWLEGYLSVLHLKAVVYVSLDNAVLGD
DKFHAKTSPLLTSLIESVLKQVDSPNHSGQTLYEQVVFTNPSWDAEVIR
PLPMDSSAYSFTAFVGVPAVEFSFMEDDQAYPFLHTKEDTYENLHKVLQ
GRLPAVAQAVAQLAGQLLIRLSHDRLLPLDFGRYGDVVLRHIGNLNEFS
GDLKARGLTLQWVYSARGDYIRAAEKLRQEIYSSEERDERLTRMYNVRI
MRVEFYFLSQYVSPADSPFRHIFMGRGDHTLGALLDHLRLLRSNSSGTP
GATSSTGFQESRFRRQLALLTWTLQGAANALSGDVWNIDNNF.

In another instance, the TfR comprises a Human TfR Protein 2 (UniProtKB-Q9UP52; TFR2_HUMAN) isoform gamma (identifier: Q9UP52-3) having an amino acid sequence of:

(SEQ ID NO: 115)
MERLWGLFQRAQQLSPRSSQTVYQRVEGPRKGHLEEEEEDGEEGAETLA
HFCPMELRGPEPLGSRPRQPNLIPWAAAGRRAAPYLVLTALLIFTGAFL
LGYVAFRGSCQACGDSVLVVSEDVNYEPDLDFHQGRLYWSDLQAMFLQF
LGEGRLEDTIRQTSLRERVAGSAGMAALTQDIRAALSRQKLDHVWTDTH
YVGLQFPDPAHPNTLHWVDEAGKVGEQLPLEDPDVYCPYSAIGNVTGEL
VYAHYGRPEDLQDLRARGVDPVGRLLLVRVGVISFAQKVTNAQDFGAQG
VLIYPEPADFSQDPPKPSLSSQQAVYGHVHLGTGDPYTPGFPSFNQTQK
LKGPVAPQEWQGSLLGSPYHLGPGPRLRLVVNNHRTSTPINNIFGCIEG
RSEPDHYVVIGAQRDAWGPGAAKSAVGTAILLELVRTFSSMVSNGFRPR
RSLLFISWDGGDFGSVGSTEWLEGYLSVLHLKAVVYVSLDNAVLGDDKF
HAKTSPLLTSLIESVLKQVDSPNHSGQTLYEQVVFTNPSWDAEVIRPLP
MDSSAYSFTAFVGVPAVEFSFMEDDQAYPFLHTKEDTYENLHKVLQGRL
PAVAQAVAQLAGQLLIRLSHDRLLPLDFGRYGDVVLRHIGNLNEFSGDL
KARGLTLQWVYSARGDYIRAAEKLRQEIYSSEERDERLTRMYNVRIMRV
EFYFLSQYVSPADSPFRHIFMGRGDHTLGALLDHLRLLRSNSSGTPGAT
SSTGFQSRFRRQLALLTWTLQGAANALSGDVWNIDNNF.

TfR can be expressed at the blood-brain barrier, the blood-cerebrospinal fluid (CSF) barrier (choroid plexus), the blood-testis barrier (Sertoli cells), the placenta (maternofetal interface), the blood-retina barrier (retinal pigment epithelium), the blood-thymus barrier (epithelial reticular cells), or another barrier. In some cases, TfR may be expressed more highly in one barrier than in other barriers. In some cases, the expression level(s) of TfR in one or more barriers can be dependent on a state of health or disease in a subject in need of treatment.

### Antibodies and Antigen-Binding Fragments

The present disclosure contemplates antibodies and antigen-binding fragments that selectively bind to TfR, e.g., SEQ ID NO: 110 or 111.

As used herein, the term “antibody” refers to an immunoglobulin (Ig), polypeptide, or a protein having a binding domain which is, or is homologous to, an antigen-binding domain. The term further includes “antigen-binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

In some instances, an antibody or an antigen-binding fragment thereof comprises an isolated antibody or antigen-binding fragment thereof, a purified antibody or antigen-binding fragment thereof, a recombinant antibody or antigen-binding fragment thereof, a modified antibody or antigen-binding fragment thereof, or a synthetic antibody or antigen-binding fragment thereof.

Antibodies and antigen-binding fragments herein can be partly or wholly synthetically produced. An antibody or antigen-binding fragment can be a polypeptide or protein having a binding domain which can be, or can be homologous to, an antigen binding domain. In one instance, an antibody or an antigen-binding fragment thereof can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. An Ig or portion thereof can, in some cases, be a human Ig. In some instances, a C_{H}3 domain can be from an immunoglobulin. In some cases, a chain or a part of an antibody or antigen binding fragment thereof, a modified antibody or antigen-binding fragment thereof, or a binding agent can be from an Ig. In such cases, an Ig can be IgG, an IgA, an IgD, an IgE, or an IgM. In cases where the Ig is an IgG, it can be a subtype of IgG, wherein subtypes of IgG can include IgG1, an IgG2a, an IgG2b, an IgG3, and an IgG4. In some cases, a CH3 domain can be from an immunoglobulin selected from the group consisting of an IgG, an IgA, an IgD, an IgE, and an IgM.

The “light chains” of antibodies (immunoglobulins) from any vertebrate species can be assigned to one of two clearly distinct types, called kappa (“κ” or “K”) or lambda (“λ”), based on the amino acid sequences of their constant domains.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.)); and (2) an approach based on crystallographic studies of antigen-antibody complexes (Al-Iazikani et al. (1997) J. Molec. Biol. 273:927-948)). As used herein, a CDR may refer to CDRs defined by either approach or by a combination of both approaches.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a β-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669).

The terms “hypervariable region” and “CDR” when used herein, refer to the amino acid residues of an antibody which are responsible for antigen-binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2), and 89-97 (CDRL3), and in the heavy chain variable domain the CDRs typically correspond to approximately residues 31-35 (CDRH1), 50-65 (CDRH2), and 95-102 (CDRH3) according to Kabat et al., Id. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2), and 91-96 (CDRL3), and in the heavy chain variable domain, the CDRs typically correspond to approximately residues 26-32 (CDRH1), 53-55 (CDRH2), and 96-101 (CDRH3) according to Chothia and Lesk, J. Mol. Biol., 196: 901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat et al., Id. As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (VH CDR1), heavy chain second complementarity determining region (VH CDR2), heavy chain third complementarity determining region (VH CDR3), light chain first complementarity determining region (VL CDR1), light chain second complementarity determining region (VL CDR2), and light chain third complementarity determining region (VL CDR3).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is generally defined to stretch from an amino acid residue at position Cys226, or from Pro230, to the carboxyl-terminus thereof. The numbering of the residues in the Fc region is that of the EU index as in Kabat et al., (Sequences of Proteins of Immunological Interest, 5th Ed. Public Health Service, National Institutes of Health, Bethesda, Md., 1991). The Fc region of an immunoglobulin generally comprises two constant domains, C_{H}2 and C_{H}3.

In one instance, a TfR antibody or antigen binding fragment can comprise variable regions. A variable region can be the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies.

In one instance, a TfR antibody or antigen binding fragment can comprise light chain regions, heavy chain regions, or light chain and heavy chain regions that confer specific binding of the molecule to Tfr. In another instance, an antibody or antigen binding fragment can comprise constant regions. A constant region can include the constant region of the antibody light chain either alone or in combination with the constant region of the antibody heavy chain.

“Antibodies” useful in the present disclosure encompass, but are not limited to, monoclonal antibodies, polyclonal antibodies, chimeric antibodies, bispecific antibodies, multispecific antibodies, heteroconjugate antibodies, humanized antibodies, human antibodies, deimmunized antibodies, mutants thereof, fusions thereof, immunoconjugates thereof, antigen-binding fragments thereof, and/or any other modified configuration of the immunoglobulin molecule that comprises an antigen recognition site of the required specificity, including glycosylation variants of antibodies, amino acid sequence variants of antibodies, and covalently modified antibodies.

“Epitope” refers to that portion of an antigen or other macromolecule capable of forming a binding interaction with the variable region binding pocket of an antibody. Such binding interactions can be manifested as an intermolecular contact with one or more amino acid residues of one or more CDRs. Antigen binding can involve, for example, a CDR3, a CDR3 pair or, in some cases, interactions of up to all six CDRs of the V_{H} and V_{L} chains. An epitope can be a linear peptide sequence (i.e., “continuous”) or can be composed of noncontiguous amino acid sequences (i.e., “conformational” or “discontinuous”). An antibody can recognize one or more amino acid sequence; therefore, an epitope can define more than one distinct amino acid sequence. Epitopes recognized by antibodies can be determined by peptide mapping and sequence analysis techniques. Binding interactions are manifested as intermolecular contacts between an epitope on an antigen and one or more amino acid residues of a CDR. Epitopes recognized by antibodies can be determined, for example, by peptide mapping or sequence analysis techniques. Binding interactions can manifest as intermolecular contacts between an epitope on an antigen and one or more amino acid residues of a complementarity determining region (CDR).

An epitope that “preferentially binds” or “selectively binds” (used interchangeably herein) to an antibody or a polypeptide is a term well understood in the art, and methods to determine such specific or preferential binding are also well known in the art. An antibody selectively binds or preferentially binds to a target if it binds with greater affinity, avidity, more readily, and/or with greater duration than it binds to other substances. For example, an antibody that specifically or preferentially binds to an epitope on a TfR represents an antibody that binds this epitope with greater affinity, avidity, more readily, and/or with greater duration than it binds to other TfR epitopes or non-TfR epitopes. An antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to specific binding means preferential binding where the affinity of the antibody, or antigen-binding fragment thereof, is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences.

In some instances, a TfR antibody is a monoclonal antibody. As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example. Other suitable methods are also within the scope of the present disclosure.

In some instances, a TfR antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antibody or an antigen binding fragment thereof described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen-binding fragments described herein can be carried out via the use of software and specific databases. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or that has been made using any suitable technique for making human antibodies. This definition of a human antibody includes antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies (Vaughan et al., 1996, Nature Biotechnology, 14:309-314; Sheets et al., 1998, PNAS USA, 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

Any of the antibodies herein can be bispecific. Bispecific antibodies are antibodies that have binding specificities for at least two different antigens and can be prepared using the antibodies disclosed herein. Methods for making bispecific antibodies are described (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature, 305, 537-539). Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690.

According to one approach to making bispecific antibodies, antibody variable domains with the desired binding specificities (antibody-antigen combining sites) are fused to immunoglobulin constant domain sequences. The fusion can be with an immunoglobulin heavy chain constant domain, comprising at least part of the hinge, CH2 and CH3 regions. The first heavy chain constant region (CH1), containing the site necessary for light chain binding, can be present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Heteroconjugate antibodies, comprising two covalently joined antibodies, are also within the scope of the disclosure. Such antibodies have been used to target immune system cells to unwanted cells (U.S. Pat. No. 4,676,980). Heteroconjugate antibodies may be made using any suitable cross-linking methods. Suitable cross-linking agents and techniques are described, for example, in U.S. Pat. No. 4,676,980.

In some instances, an antibody herein is a chimeric antibody. “Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin, is inserted in place of the murine Fc.

Chimeric or hybrid antibodies also may be prepared in vitro using suitable methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Provided herein are antibodies and antigen-binding fragments thereof, modified antibodies and antigen-binding fragments thereof, and binding agents that specifically bind to one or more epitopes on one or more target antigens. In one instance, a binding agent selectively binds to an epitope on a single antigen. In another instance, a binding agent is bivalent and either selectively binds to two distinct epitopes on a single antigen or binds to two distinct epitopes on two distinct antigens. In another instance, a binding agent is multivalent (i.e., trivalent, quatravalent, etc.) and the binding agent binds to three or more distinct epitopes on a single antigen or binds to three or more distinct epitopes on two or more (multiple) antigens.

Functional fragments of any of the antibodies herein are also contemplated. The terms “antigen-binding portion of an antibody,” “antigen-binding fragment,” “antigen-binding domain,” “antibody fragment,” or a “functional fragment of an antibody” are used interchangeably herein to refer to one or more fragments of an antibody that retain the ability to specifically bind to an antigen. Representative antigen-binding fragments include, but are not limited to, a Fab, a Fab′, a F(ab′)₂, a Fv, a scFv, a dsFv, a variable heavy domain, a variable light domain, a variable NAR domain, bi-specific scFv, a bi-specific Fab₂, a tri-specific Fab₃, an AVIMER®, a minibody, a diabody, a maxibody, a camelid, a VHH, a minibody, an intrabody, fusion proteins comprising an antibody portion (e.g., a domain antibody), and a single chain binding polypeptide.

“F(ab′)₂” and “Fab′” moieties can be produced by treating an Ig with a protease such as pepsin and papain, and include antibody fragments generated by digesting immunoglobulin near the disulfide bonds existing between the hinge regions in each of the two heavy chains. For example, papain cleaves IgG upstream of the disulfide bonds existing between the hinge regions in each of the two heavy chains to generate two homologous antibody fragments in which an light chain composed of V_{L} and C_{L} (light chain constant region), and a heavy chain fragment composed of V_{H} and C_{Hγ1} (γ1) region in the constant region of the heavy chain) are connected at their C terminal regions through a disulfide bond. Each of these two homologous antibody fragments is called Fab′. Pepsin also cleaves IgG downstream of the disulfide bonds existing between the hinge regions in each of the two heavy chains to generate an antibody fragment slightly larger than the fragment in which the two above-mentioned Fab′ are connected at the hinge region. This antibody fragment is called F(ab′)₂.

The Fab fragment also contains the constant domain of the light chain and the first constant domain (C_{H}1) of the heavy chain. Fab′ fragments differ from Fab fragments by the addition of a few residues at the carboxyl terminus of the heavy chain C_{H}1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

A “Fv” as used herein refers to an antibody fragment which contains a complete antigen-recognition and antigen-binding site. This region consists of a dimer of one heavy chain and one light chain variable domain in tight, non-covalent or covalent association (disulfide linked Fvs have been described, see, e.g., Reiter et al. (1996) Nature Biotechnology 14:1239-1245). It is in this configuration that the three CDRs of each variable domain interact to define an antigen-binding site on the surface of the VH-V_{L} dimer. Collectively, a combination of one or more of the CDRs from each of the V_{H} and V_{L} chains confer antigen-binding specificity to the antibody. For example, it would be understood that, for example, the CDRH3 and CDRL3 could be sufficient to confer antigen-binding specificity to an antibody when transferred to V_{H} and V_{L} chains of a recipient antibody or antigen-binding fragment thereof and this combination of CDRs can be tested for binding, specificity, affinity, etc. using any of the techniques described herein. Even a single variable domain (or half of an Fv comprising only three CDRs specific for an antigen) has the ability to recognize and bind antigen, although likely at a lower specificity or affinity than when combined with a second variable domain. Furthermore, although the two domains of a Fv fragment (V_{L} and VH) are coded for by separate genes, they can be joined using recombinant methods by a synthetic linker that enables them to be made as a single protein chain in which the V_{L} and VH regions pair to form monovalent molecules (known as single chain Fv (scFv); Bird et al. (1988) Science 242:423-426; Huston et al. (1988) Proc. Natl. Acad. Sci. USA 85:5879-5883; and Osbourn et al. (1998) Nat.

Biotechnol. 16:778). Such scFvs are also intended to be encompassed within the term “antigen-binding portion” of an antibody. Any V_{H} and V_{L} sequences of specific scFv can be linked to an Fc region cDNA or genomic sequences in order to generate expression vectors encoding complete Ig (e.g., IgG) molecules or other isotypes. V_{H} and V_{L} can also be used in the generation of Fab, Fv, or other fragments of Igs using either protein chemistry or recombinant DNA technology.

“Single-chain Fv” or “sFv” antibody fragments comprise the V_{H} and V_{L} domains of an antibody, wherein these domains are present in a single polypeptide chain. In some embodiments, the Fv polypeptide further comprises a polypeptide linker between the V_{H} and V_{L} domains which enables the sFv to form the desired structure for antigen binding. For a review of sFvs, see, e.g., Pluckthun in The Pharmacology of Monoclonal Antibodies, Vol. 113, Rosenburg and Moore eds. Springer-Verlag, New York, pp. 269-315 (1994).

The term “AVIMER®” refers to a class of therapeutic proteins of human origin, which are unrelated to antibodies and antibody fragments, and are composed of several modular and reusable binding domains, referred to as A-domains (also referred to as class A module, complement type repeat, or LDL-receptor class A domain). They were developed from human extracellular receptor domains by in vitro exon shuffling and phage display (Silverman et al., 2005, Nat. Biotechnol. 23:1493-1494; Silverman et al., 2006, Nat. Biotechnol. 24:220). The resulting proteins can contain multiple independent binding domains that can exhibit improved affinity and/or specificity compared with single-epitope binding proteins. Each of the known 217 human A-domains comprises ˜35 amino acids (˜4 kDa); and these domains are separated by linkers that average five amino acids in length. Native A-domains fold quickly and efficiently to a uniform, stable structure mediated primarily by calcium binding and disulfide formation. A conserved scaffold motif of only 12 amino acids is required for this common structure. The end result is a single protein chain containing multiple domains, each of which represents a separate function. Each domain of the proteins binds independently, and the energetic contributions of each domain are additive.

Antigen-binding polypeptides also include heavy chain dimers such as, for example, antibodies from camelids and sharks. Camelid and shark antibodies comprise a homodimeric pair of two chains of V-like and C-like domains (neither has a light chain). Since the VH region of a heavy chain dimer IgG in a camelid does not have to make hydrophobic interactions with a light chain, the region in the heavy chain that normally contacts a light chain is changed to hydrophilic amino acid residues in a camelid. VH domains of heavy-chain dimer IgGs are called V_{HH} domains. Shark Ig-NARs comprise a homodimer of one variable domain (termed a V-NAR domain) and five C-like constant domains (C-NAR domains). In camelids, the diversity of antibody repertoire is determined by the CDRs 1, 2, and 3 in the V_{H} or V_{HH} regions. The CDR3 in the camel V_{HH} region is characterized by its relatively long length, averaging 16 amino acids (Muyldermans et al., 1994, Protein Engineering 7(9): 1129). This is in contrast to CDR3 regions of antibodies of many other species. For example, the CDR3 of mouse VH has an average of 9 amino acids. Libraries of camelid-derived antibody variable regions, which maintain the in vivo diversity of the variable regions of a camelid, can be made by, for example, the methods disclosed in U.S. Patent Application Ser. No. 20050037421.

As used herein, a “maxibody” refers to a bivalent scFv covalently attached to the Fc region of an immunoglobulin, see, e.g., Fredericks et al., Protein Engineering, Design & Selection, 17:95-106 (2004) and Powers et al., Journal of Immunological Methods, 251:123-135 (2001).

As used herein, a “dsFv” can be a Fv fragment obtained by introducing a Cys residue into a suitable site in each of a heavy chain variable region and a light chain variable region, and then stabilizing the heavy chain variable region and the light chain variable region by a disulfide bond. The site in each chain, into which the Cys residue is to be introduced, can be determined based on a conformation predicted by molecular modeling. In the present disclosure, for example, a conformation is predicted from the amino acid sequences of the heavy chain variable region and light chain variable region of the above-described antibody, and DNA encoding each of the heavy chain variable region and the light chain variable region, into which a mutation has been introduced based on such prediction, is then constructed. The DNA construct is incorporated then into a suitable vector and prepared from a transformant obtained by transformation with the aforementioned vector.

Single chain variable region fragments (“scFv”) of antibodies are described herein. Single chain variable region fragments may be made by linking light and/or heavy chain variable regions by using a short linking peptide. Bird et al. (1988) Science 242:423-426. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect, or mammalian cells, or prokaryotic, such as E. coli. Polynucleotides encoding the scFv of interest can be made by routine manipulations such as ligation of polynucleotides. The resultant scFv can be isolated using any suitable protein purification techniques.

Diabodies can be single chain antibodies. Diabodies can be bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al., Proc. Natl. Acad. Sci. USA, 90:6444-6448 (1993); and Poljak, R. J., et al., Structure, 2:1121-1123 (1994)).

As used herein, a “minibody” refers to a scFv fused to CH3 via a peptide linker (hingeless) or via an IgG hinge has been described in Olafsen, et al., Protein Eng Des Sel. April 2004; 17(4):315-23.

As used herein, an “intrabody” refers to a single chain antibody which demonstrates intracellular expression and can manipulate intracellular protein function (Biocca, et al., EMBO J. 9:101-108, 1990; Colby et al., Proc Natl Acad. Sci. USA. 101:17616-21, 2004). Intrabodies, which comprise cell signal sequences which retain the antibody construct in intracellular regions, may be produced as described in Mhashilkar et al., (EMBO J., 14:1542-51, 1995) and Wheeler et al. (FASEB J. 17:1733-5. 2003). Transbodies are cell-permeable antibodies in which a protein transduction domains (PTD) is fused with single chain variable fragment (scFv) antibodies Heng et al. (Med Hypotheses. 64:1105-8, 2005).

Suitable linkers may be used to multimerize binding agents. A non-limiting example of a linking peptide is (GGGGS)₃ (SEQ ID NO: 113), which bridges approximately 3.5 nm between the carboxy terminus of one variable region and the amino terminus of the other variable region. Linkers of other sequences have been designed and used. Bird, et al. (Id.) Linkers can in turn be modified for additional functions, such as attachment of drugs or attachment to solid supports.

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as binding affinity (K_{D}). In some cases, K_{D} can be represented as a ratio of k_{off}, which can refer to the rate constant for dissociation of an antibody from the antibody or antigen-binding fragment/antigen complex, to kₒₙ, which can refer to the rate constant for association of an antibody or antigen binding fragment to an antigen. Binding affinity may be determined using methods including, for example, surface plasmon resonance (SPR; Biacore), Kinexa Biosensor, scintillation proximity assays, enzyme-linked immunosorbent assay (ELISA), ORIGEN immunoassay (IGEN), fluorescence quenching, fluorescence transfer, yeast display, or any combination thereof. Binding affinity may also be screened using a suitable bioassay.

In some cases, the K_{D} value may be important factor regarding whether or not an antibody can successfully cross a blood-tissue barrier. In some cases, a mutation in the TfR gene of a subject can alter the K_{D} of such an antibody in that subject. In some cases, different species might express TfR proteins which display affinity to antibodies and antigen-binding fragments differently. Thus, for different subjects or types of subjects, different antibodies or antigen-binding fragments can be used to ensure a proper affinity for successful transport and subsequent dissociation. The K_{D} of a binding agent, or an antibody or antigen-binding fragment described herein can be between about 1 nM and 5 μM.

Also provided herein are affinity matured antibodies. For example, affinity matured antibodies can be produced by any suitable procedure (see, e.g., Marks et al., 1992, Bio/Technology, 10:779-783; Barbas et al., 1994, Proc Nat. Acad. Sci, USA 91:3809-3813; Schier et al., 1995, Gene, 169:147-155; Yelton et al., 1995, J. Immunol., 155:1994-2004; Jackson et al., 1995, J. Immunol., 154(7):3310-9; Hawkins et al, 1992, J. Mol. Biol., 226:889-896; and WO2004/058184). The following methods may be used for adjusting the affinity of an antibody and for characterizing a CDR. One way of characterizing a CDR of an antibody and/or altering (such as improving) the binding affinity of a polypeptide, such as an antibody, is termed “library scanning mutagenesis.” Generally, library scanning mutagenesis works as follows. One or more amino acid position in the CDR is replaced with two or more (such as 3, 4, 5, 6, 7, 8, 9, 10, 11, 12, 13, 14, 15, 16, 17, 18, 19, or 20) amino acids. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, for example, about 20-80 clones (depending on the complexity of the library), from each library can be screened for binding specificity or affinity to the target polypeptide (or other binding target), and candidates with increased, the same, decreased, or no binding are identified. Binding affinity may be determined using Biacore surface plasmon resonance analysis, which detects differences in binding affinity of about 2-fold or greater. Biacore can be particularly useful when the starting antibody already binds with a relatively high affinity, for example, a K_{D} of about 10 nM or lower.

In some instances, an antibody or antigen binding fragment is bi-specific or multi-specific and can specifically bind to more than one antigen. In some cases, such a bi-specific or multi-specific antibody or antigen binding fragment can specifically bind to 2 or more different antigens. In some cases, a bi-specific antibody or antigen-binding fragment can be a bivalent antibody or antigen-binding fragment. In some cases, a multi specific antibody or antigen-binding fragment can be a bivalent antibody or antigen-binding fragment, a trivalent antibody or antigen-binding fragment, or a quatravalent antibody or antigen-binding fragment.

An antibody or antigen binding fragment described herein can be an isolated, purified, recombinant, or synthetic.

Methods of Making and Expressing Antibodies

The antibodies described herein may be made by any suitable method. Antibodies can often be produced in large quantities, particularly when utilizing high level expression vectors. Techniques, such as those provided and incorporated herein, can be used to modify nucleotides encoding amino acid sequences using recombinant techniques in restriction endonuclease sites.

In one instance, when an animal (e.g., a mouse, rat, rabbit, primate, etc.) is utilized to make an antibody, the route and schedule of immunization of the host animal with the target antigen are generally in keeping with established and conventional techniques for antibody stimulation and production, as further described herein.

It is contemplated that any mammalian subject including humans or antibody producing cells therefrom can be manipulated to serve as the basis for production of mammalian, including human, hybridoma cell lines. Typically, the host animal is inoculated intraperitoneally, intramuscularly, orally, subcutaneously, intraplantar, and/or intradermally with an amount of immunogen, including as described herein.

Immunization of a host animal with a human protein, or a fragment containing a target amino acid sequence conjugated to an adjuvant that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or any other suitable adjuvant can yield a population of antibodies.

Hybridomas can be prepared from the lymphocytes of immunized animals and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines may be used in the hybridization. Generally, the technique involves fusing myeloma cells and lymphoid cells using a fusogen such as polyethylene glycol, or by electrical means. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce monoclonal antibodies. The hybridomas are expanded and subcloned, if desired, and supernatants are assayed for anti-immunogen activity by conventional immunoassay procedures (e.g., radioimmunoassay, enzyme immunoassay, fluorescence immunoassay, etc.). Hybridomas that may be used as source of antibodies encompass all derivatives, progeny cells of the parent hybridomas that produce monoclonal antibodies, or a portion thereof. Hybridomas that produce such antibodies may be grown in vitro or in vivo using known procedures. The monoclonal antibodies may be isolated from the culture media or body fluids by conventional immunoglobulin purification procedures such as ammonium sulfate precipitation, gel electrophoresis, dialysis, chromatography, and ultrafiltration, if desired.

Undesired activity, if present, can be removed, for example, by running the preparation over adsorbents made of the immunogen attached to a solid phase and eluting or releasing the desired antibodies off the immunogen.

Antibodies may be made recombinantly and expressed using any suitable method. Antibodies may be made recombinantly by phage display technology. For example, U phage display technology can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” In this method, the affinity of “primary” human antibodies obtained by phage display can be improved by sequentially replacing the heavy and light chain V region genes with repertoires of naturally-occurring variants (repertoires) of V domain genes obtained from unimmunized donors. This technique allows the production of antibodies and antibody fragments with affinities in the pM-nM range. Another strategy for making antibodies includes, for example, large phage antibody repertoires.

Gene shuffling can also be used to derive human antibodies from rodent antibodies, where the human antibody has similar affinities and specificities to the starting rodent antibody. According to this method, which is also referred to as “epitope imprinting,” the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained. Unlike traditional humanization of rodent antibodies by CDR grafting, this technique can provide completely human antibodies, which have no framework or CDR residues of rodent origin.

There are four general steps to humanize a monoclonal antibody. These are: (1) determining the nucleotide and predicted amino acid sequence of the starting antibody light and heavy variable domains, (2) designing the humanized antibody, i.e., deciding which antibody framework region to use during the humanizing process, (3) the actual humanizing methodologies/techniques, and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; and 6,180,370. A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349:293-299 (1991), Lobuglio et al. Proc. Nat. Acad. Sci. USA 86:4220-4224 (1989), Shaw et al. J. Immunol. 138:4534-4538 (1987), and Brown et al. Cancer Res. 47:3577-3583 (1987).

Other references describe rodent CDRs grafted into a human supporting framework region (FR) prior to fusion with an appropriate human antibody constant domain. See, for example, Riechmann et al. Nature 332:323-327 (1988), Verhoeyen et al., Science, 239:1534-1536 (1988), and Jones et al., Nature, 321:522-525 (1986). Another reference describes rodent CDRs supported by recombinantly veneered rodent framework regions. See, e.g., European Patent Publication No. 0519596. These “humanized” molecules are designed to minimize unwanted immunological response toward rodent anti-human antibody molecules which limits the duration and effectiveness of therapeutic applications of those moieties in human recipients. For example, the antibody constant region can be engineered such that it is immunologically inert (e.g., does not trigger complement lysis). See, e.g., PCT Publication No. WO 99/058572; and UK Patent Application No. 9809951.8.

Other methods of humanizing antibodies that may also be utilized are disclosed by Daugherty et al., Nucl. Acids Res., 19:2471-2476 (1991) and in U.S. Pat. Nos. 6,180,377; 6,054,297; 5,997,867; 5,866,692; 6,210,671; and 6,350,861; and in PCT Publication No. WO 01/27160.

In yet another alternative, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are XENOMOUSE™ from Abgenix, Inc. (Fremont, Calif.) and HUMAB-MOUSE® and TC MOUSE™ from Medarex, Inc. (Princeton, N.J.).

It will be apparent that although the above discussion pertains to humanized antibodies, the general principles discussed are applicable to customizing antibodies for use, for example, in dogs, cats, primates, equines, and bovines. It is further apparent that one or more aspects of humanizing an antibody described herein may be combined, e.g., CDR grafting, framework mutation, and CDR mutation.

If desired, an antibody of interest may be sequenced using any known method and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the specificity, affinity, or other characteristics of the antibody. For example, the constant region may be engineered to more resemble human constant regions to avoid immune response if the antibody is used in clinical trials and treatments in humans.

Also provided herein are methods of making any of these antibodies or polypeptides. The polypeptides can be produced by proteolytic or other degradation of the antibodies, by recombinant methods (i.e., single or fusion polypeptides) as described above, or by chemical synthesis. Polypeptides of the antibodies, especially shorter polypeptides up to about 50 amino acids, can be made by chemical synthesis. Methods of chemical synthesis are commercially available. For example, an antibody could be produced by an automated polypeptide synthesizer employing a solid phase method.

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. Methods for making derivatives of antibodies, e.g., single chain, etc. are also within the scope of the present disclosure.

As used herein, “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected with a polynucleotide(s) of this disclosure.

DNA encoding an antibody may be readily isolated and sequenced using conventional procedures (e.g., by using oligonucleotide probes that are capable of binding specifically to genes encoding the heavy and light chains of the monoclonal antibodies). Hybridoma cells may serve as a source of such DNA. Once isolated, the DNA may be placed into one or more expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, or by covalently joining to the immunoglobulin coding sequence all or part of the coding sequence for a non-immunoglobulin polypeptide. In that manner, “chimeric” or “hybrid” antibodies are prepared that have the binding specificity of an antibody described herein.

Contemplated herein are vectors that encode the one or more antibodies or antigen-binding fragments described herein. As used herein, “vector” means a construct, which is capable of delivering, and possibly expressing, one or more gene(s) or sequence(s) of interest in a host cell. Examples of vectors include, but are not limited to, viral vectors; naked DNA or RNA expression vectors; plasmid, cosmid, or phage vectors; DNA or RNA expression vectors associated with cationic condensing agents; DNA or RNA expression vectors encapsulated in liposomes; and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

An expression vector can be used to direct expression of an antibody. Expression vectors can be administered to obtain expression of an exogenous protein in vivo. See, e.g., U.S. Pat. Nos. 6,436,908; 6,413,942; and 6,376,471.

For high level production, a widely used mammalian expression system is one which utilizes Lonza's GS Gene Expression System™. This system uses a viral promoter and selection via glutamine metabolism to provide development of high-yielding and stable mammalian cell lines.

For alternative high-level production, a widely used mammalian expression system is one which utilizes gene amplification by dihydrofolate reductase deficient (“dhfr-”) Chinese hamster ovary cells. The system is based upon the dihydrofolate reductase “dhfr” gene, which encodes the DHFR enzyme, which catalyzes conversion of dihydrofolate to tetrahydrofolate. In order to achieve high production, dhfr− CHO cells are transfected with an expression vector containing a functional DHFR gene, together with a gene that encodes a desired protein. In this case, the desired protein is recombinant antibody heavy chain and/or light chain.

By increasing the amount of the competitive DHFR inhibitor methotrexate (MTX), the recombinant cells develop resistance by amplifying the dhfr gene. In standard cases, the amplification unit employed is much larger than the size of the dhfr gene, and as a result the antibody heavy chain is co-amplified.

When large scale production of the protein, such as the antibody chain, is desired, both the expression level and the stability of the cells being employed are taken into account.

The present application provides an isolated polynucleotide (nucleic acid) encoding an antibody or portion thereof as described herein, vectors containing such polynucleotides, and host cells and expression systems for transcribing and translating such polynucleotides into polypeptides.

The present application also provides constructs in the form of plasmids, vectors, transcription or expression cassettes which comprise at least one polynucleotide as above.

The present application also provides a recombinant host cell which comprises one or more constructs as above. A nucleic acid encoding any antibody described herein forms an aspect of the present application, as does a method of production of the antibody, which method comprises expression from encoding nucleic acid therefrom. Expression can be achieved by culturing under appropriate conditions recombinant host cells containing the nucleic acid. Following production by expression, an antibody or a portion thereof can be isolated and/or purified using any suitable technique, then used as appropriate.

Systems for cloning and expression of a polypeptide in a variety of different host cells are contemplated for use herein.

A further aspect provides a host cell containing nucleic acid as disclosed herein using any suitable method. A still further aspect provides a method comprising introducing such nucleic acid into a host cell. The introduction can be followed by causing or allowing expression from the nucleic acid, e.g., by culturing host cells under conditions for expression of the gene.

A polynucleotide encoding an antibody or a portion thereof can be prepared recombinantly/synthetically in addition to, or rather than, cloned. In a further embodiment, the full DNA sequence of the recombinant DNA molecule or cloned gene of an antibody or portion thereof described herein can be operatively linked to an expression control sequence which can be introduced into an appropriate host using any suitable method.

DNA sequences can be expressed by operatively linking them to an expression control sequence in an appropriate expression vector and employing that expression vector to transform an appropriate host cell. Any of a wide variety of expression control sequences—sequences that control the expression of a DNA sequence operatively linked to it—can be used in these vectors to express the DNA sequences.

A wide variety of host/expression vector combinations can be employed in expressing the DNA sequences of this disclosure. It will be understood that not all vectors, expression control sequences, and hosts will function equally well to express the DNA sequences. Neither will all hosts function equally well with the same expression system. In some embodiments, in selecting a vector, the host is considered such that the vector can function in it. The vector's copy number, the ability to control that copy number, and the expression of any other proteins encoded by the vector, such as antibiotic markers, may also be considered. In certain embodiments, in selecting a vector, the host is considered such that the vector functions in it. The vector's copy number, the ability to control that copy number, and the expression of any other proteins encoded by the vector, such as antibiotic markers, can also be considered.

The present application also provides a method which comprises using a construct as stated above in an expression system in order to express the antibodies (or portions thereof) as above. Considering these and other factors, a variety of vector/expression control sequence/host combinations can be constructed that can express the DNA sequences on fermentation or in large scale animal culture.

Simultaneous incorporation of the antibody (or portion thereof)-encoding nucleic acids and the selected amino acid position changes can be accomplished by a variety of suitable methods including, for example, recombinant and chemical synthesis.

Isolation, Purification, and Detection

Specific nucleic acid molecules and vectors that encode binding agents described herein can be isolated and/or purified from their natural environment in substantially pure or homogeneous form. Methods of purifying proteins and nucleic acids are contemplated for use herein. “Isolated” (used interchangeably with “substantially pure”) when applied to polypeptides means a polypeptide or a portion thereof which, by virtue of its origin or manipulation: (i) is present in a host cell as the expression product of a portion of an expression vector; (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically or (ii) expressed in a host cell and purified away from associated and contaminating proteins. The term generally means a polypeptide that has been separated from other proteins and nucleic acids with which it naturally occurs. The polypeptide may also be separated from substances such as antibodies or gel matrices (polyacrylamide) which are used to purify it.

Polypeptides can be isolated and purified from culture supernatant or ascites by saturated ammonium sulfate precipitation, an euglobulin precipitation method, a caproic acid method, a caprylic acid method, ion exchange chromatography (DEAE or DE52), or affinity chromatography using anti-Ig column or a protein A, protein G, or protein L column such as described in more detail below. In one aspect, reference to a binding agent, an antibody or an antigen-binding fragment thereof also refers to an “isolated binding agent,” an “isolated antibody,” or an “isolated antigen-binding fragment.” In another aspect, reference to a binding agent, an antibody, or an antigen-binding fragment thereof also refers to a “purified binding agent,” a “purified antibody,” or a “purified antigen-binding fragment.”

In addition to the therapeutic methods described herein, binding agents, antibodies, or antigen-binding fragments thereof that specifically bind to TfR can also be used for purification and/or to detect TfR levels in a sample or subject. Compositions of antibodies and antigen-binding fragments described herein can be used as non-therapeutic agents (e.g., as affinity purification agents). Generally, in one such embodiment, a protein of interest can be immobilized on a solid phase such a Sephadex resin or filter paper. The immobilized protein can be contacted with a sample containing the target of interest (or fragment thereof) to be purified, and thereafter the support can be washed with a suitable solvent that will remove substantially all the material in the sample except the target protein, which is bound to the immobilized antibody. Finally, the support can be washed with another suitable solvent, such as glycine buffer, pH 5.0, which will release the target protein.

A sample may be obtained from a subject and optionally treated for use in a particular assay. The sample is contacted with a binding agent, antibody, or antigen-binding fragment thereof that specifically bind to TfR, and the presence of a TfR in the sample is identified when detection of the binding agent, antibody, or antigen-binding fragment thereof is observed. The term sample is used in its broadest sense. A “biological sample” as used herein, includes, but is not limited to, any quantity of a substance from a living thing or formerly living thing such as, for example, humans, mice, rats, monkeys, dogs, rabbits, and other animals. Such samples include, but are not limited to, blood, serum, urine, synovial fluid, cells, organs, tissues, bone marrow, lymph nodes, and neurons. In one instance, the binding agent, antibody, or antigen-binding fragment thereof, that specifically bind to TfR, may be labeled with, for example, biotin, such that addition of a secondary agent such as, for example, streptavidin alkaline phosphatase (AP), may enhance signal detection in an assay.

Assays that may be utilized in detection methods include, but are not limited to, ELISA, ELISPOT, western Blot, FACS, flow cytometry, immunohistochemistry, etc.

Characterization of Anti-TfR antibodies

Anti-TfR antibodies and antigen-binding fragments thereof can be identified or characterized.

Antibodies may be characterized using suitable methods. For example, one method is to identify the epitope to which it binds, or “epitope mapping.” Methods for mapping and characterizing the location of epitopes on proteins include, but are not limited to, solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an anti-TfR antibody binds. Epitope mapping is commercially available from various sources, for example, Pepscan Systems (Edelhertweg 15, 8219 PH Lelystad, The Netherlands). The epitope can be a linear epitope, i.e., contained in a single stretch of amino acids, or a conformational epitope formed by a three-dimensional interaction of amino acids that may not necessarily be contained in a single stretch. Peptides of varying lengths (e.g., at least 4-6 amino acids long) can be isolated or synthesized (e.g., recombinantly) and used for binding assays with an anti-TfR antibody.

In another example, the epitope to which the anti-TfR antibody binds can be determined in a systematic screening by using overlapping peptides derived from the anti-TfR sequence and determining binding by the anti-TfR antibody. According to the gene fragment expression assays, the open reading frame encoding TfR is fragmented either randomly or by specific genetic constructions and the reactivity of the expressed fragments of TfR with the antibody to be tested is determined. The gene fragments may, for example, be produced by PCR and then transcribed and translated into protein in vitro, in the presence of radioactive amino acids. The binding of the antibody to the radioactively labeled TfR fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant TfR in which various fragments of the TfR polypeptide have been replaced (swapped) with sequences from a closely related, but antigenically distinct protein. By assessing binding of the antibody to the mutant TfR, the importance of the particular TfR fragment to antibody binding can be assessed.

Yet another method which can be used to characterize an anti-TfR antibody is to use competition assays with other antibodies known to bind to the same antigen, i.e., various fragments on TfR, to determine if the anti-TfR antibody binds to the same epitope as other antibodies.

In vitro assays such as, for example, immunohistochemistry, FACS, western Blots, ELISPOTs, etc., can be utilized to show that an antibody, or antigen-binding fragment thereof is able to bind to a TfR.

In vivo assays such as, for example, those described herein below in the Examples, can be utilized to demonstrate that an antibody, or antigen-binding fragment thereof is able to bind to a TfR.

### Representative Anti-TfR Antibody or Antigen-Binding Fragment Sequences

An antibody or antigen-binding fragment thereof described herein can have a variable light (VL) chain that selectively binds to a TfR.

Representative Variable Light Chain Sequences

An antibody or antigen-binding fragment thereof, described herein can comprise a VL framework (FR) 1 (FR1) having an amino acid sequence that is at least 80%, 85%, 90%, 91%, 92%, 93%, 94% 95%, 96%, 97%, 98%, or 99% identical to X₁IX₂MTQSPX₃X₄LX₅X₆SX₇GXsRX₉TX₁₀X₁₁C (SEQ ID NO: 9), wherein X₁ comprises D or E; X₂ comprises Q or V; X₃ comprises S, D, or A; X₄ comprises S or T; X₅ comprises S or A; X₆ comprises A or V; X₇ comprises L or P; X₈ comprises D or E; X₉ comprises V or A, X₁₀ comprises I or L; and X₁₁ comprises T, N, or S. In one instance, the VL FR1 comprises one of the following sequences:

| VL FR1 | SEQ ID NO: |
|---|---|
| DIQMTQSPSSLSASVGDRVTITC | 10 |
| DIVMTQSPDSLAVSLGERATINC | 11 |
| EIVMTQSPATLSVSPGERATLSC | 12 |

An antibody or antigen-binding fragment thereof described herein can comprise a VL CDR1 having an amino acid sequence that is at least 80%, 85%, 90%, 91%, 92%, 93%, 94% 95%, 96%, 97%, 98%, or 99% identical to RASQTLYTNYLA (SEQ ID NO: 26); KSSRSVLRTSKNKNFLA (SEQ ID NO: 27); or X₁ASX₂X₃X₄X₅X₆X₇LX₈ (SEQ ID NO: 13), wherein X₁ comprises R or Q; X₂ comprises Q or R; X₃ comprises G, D, S or N; X₄ comprises I or V; X₅ comprises S, R, G, N or K; X₆ comprises R, K, S, G, or D; X₇ comprises N, W, Y, A, R, or K; and X₈ comprises A or N. In one instance, the VL CDR1 comprises one of the following sequences:

| | SEQ | | SEQ |
|---|---|---|---|
| | ID | | ID |
| VL CDR2 | NO: | VL CDR2 | NO: |
| RASRGISRWLA | 14 | RASQNINKNLN | 21 |
| QASQDIIDSLN | 15 | RASQNIGSRLN | 22 |
| RASQDIRRYLA | 16 | RASRSISDYLA | 23 |
| RASRGVSKWLA | 17 | RASQNIKRYLN | 24 |
| RASRGVSSWLA | 18 | RASQSVRRKLA | 25 |
| RASRSVGGALA | 19 | RASQTLYTNYLA | 26 |
| RASQSIRRYLN | 20 | KSSRSVLRTSKNKNFLA | 27 |

An antibody or antigen-binding fragment thereof, described herein can comprise a VL FR2 having an amino acid sequence that is at least 80%, 85%, 90%, 91%, 92%, 93%, 94% 95%, 96%, 97%, 98%, or 99% identical to WYQQKPGX₁X₂PX₃LLIY (SEQ ID NO: 28), wherein X₁ comprises Q or K; X₂ comprises A or P; and X₃ comprises R or K. In one instance, the VL FR2 comprises one of the following sequences:

| VL FR1 | SEQ ID NO: |
|---|---|
| WYQQKPGQAPRLLIY | 29 |
| WYQQKPGKAPKLLIY | 30 |
| WYQQKPGQPPKLLIY | 31 |

An antibody or antigen-binding fragment thereof, described herein can comprise a VL CDR2 having an amino acid sequence that is at least 80%, 85%, 90%, 91%, 92%, 93%, 94% 95%, 96%, 97%, 98%, or 99% identical to X₁X₂X₃X₄X₅ X₆X₇ (SEQ ID NO: 32), wherein X₁ comprises G, A, K, W, or S; X₂ comprises A or T; X₃ comprises F or S, X₄ comprises T, R, S, or N; X₅ comprises R or L; X₆ comprises R, Q, A, or E; and X₇ comprises S, N, or T. In one instance, the VL CDR2 comprises one of the following sequences:

| VL CDR2 | SEQ ID NO: |
|---|---|
| GASTRAT | 33 |
| AAFRLRS | 34 |
| AASSLQS | 35 |
| KASRLQS | 36 |
| AASTLQS | 37 |
| WASTRES | 38 |
| KASSLAN | 39 |
| KASSLES | 40 |
| STSNLQS | 41 |
| KASRLET | 42 |

In one instance, an antibody or antigen-binding fragment thereof, described herein comprises a VL FR3 having an amino acid sequence that is at least 80%, 85%, 90%, 91%, 92%, 93%, 94% 95%, 96%, 97%, 98%, or 99% identical to GX₁PX₂RFSGSGSGTX₃FTLTISSLQX₄EDX₅AX₆YY (SEQ ID NO: 43), wherein X₁ comprises I or V; X₂ comprises A, D, or S; X₃ comprises E or D; X₄ comprises S. P, or A; X₅ comprises F or V; and X₆ comprises V or T. In one instance, the VL FR3 comprises one of the following sequences:

| VL FR3 | SEQ ID NO: |
|---|---|
| GIPARFSGSGSGTEFTLTISSLQSEDFAVYY | 44 |
| GVPSRFSGSGSGTDFTLTISSLQPEDFATYY | 45 |
| GVPDRFSGSGSGTDFTLTISSLQAEDVAVYY | 46 |

An antibody or antigen-binding fragment thereof, described herein can comprise a VL CDR3 VL CDR3 having an amino acid sequence that is at least 80%, 85%, 90%, 91%, 92%, 93%, 94% 95%, 96%, 97%, 98%, or 99% identical to CQX₁X₂X₃X₄X₅PX₆TF (SEQ ID NO: 47), wherein X₁ comprises Q or K; X₂ comprises S, A, G, Y, H; X₃ comprises Y, N, F, K, G, or L; X₄ comprises K, S, or R; X₅ comprises T, F, Y, A, L, R, P, or S; and X₆ comprises Y, W, F, R, L, or I. In one instance, the VL CDR3 comprises one of the following sequences:

| VL CDR3 | SEQ ID NO: |
|---|---|
| CQQSYKTPYTF | 48 |
| CQQAYSFPWTF | 49 |
| CQQGYSTPFTF | 50 |
| CQQYNSYPRTF | 51 |
| CQQYYSTPFTF | 52 |
| CQQYFSAPLTF | 53 |
| CQKYNSAPLTF | 54 |
| CQQAKSLPLTF | 55 |
| CQQYKSRPLTF | 56 |
| CQQHGSPPFTF | 57 |
| CQQSYSTPLTF | 58 |
| CQQYLRSPITF | 59 |

An antibody or antigen-binding fragment thereof, described herein can comprise a VL FR4 VL FR4 having an amino acid sequence that is at least 80%, 85%, 90%, 91%, 92%, 93%, 94% 95%, 96%, 97%, 98%, or 99% identical to GXGTX₂X₃X₄IK (SEQ ID NO: 60), wherein X₁ comprises G, Q, or P; X₂ comprises K or R; X₃ comprises L or V; and X₄ comprises E or D. In one instance, the VL FR4 comprises one of the following sequences:

| VL FR4 | SEQ ID NO: |
|---|---|
| GQGTKVEIK | 61 |
| GQGTKLEIK | 62 |
| GPGTKVDIK | 63 |

An antibody an antibody or antigen-binding fragment thereof described herein comprises a heavy chain. In one instance, an antibody or antigen-binding fragment thereof described herein comprises a heavy chain CDR3 encoded by a nucleic acid sequence that is at least 80%, 85%, 90%, 91%, 92%, 93%, 94% 95%, 96%, 97%, 98%, or 99% identical to

(SEQ ID NO: 8)
TGTGCGAAAGGCGGGCGCGATGGGTATAAGGGCTACTTTGACTACTGG.

In one instance, an antibody or antigen-binding fragment thereof described herein comprises a heavy chain FR1 having an amino acid sequence that is at least 80%, 85%, 90%, 91%, 92%, 93%, 94% 95%, 96%, 97%, 98%, or 99% identical to EVQLLESGGGLVQPGGSLRLSCAASG (SEQ ID NO: 1). In one instance, an antibody or antigen-binding fragment thereof described herein comprises a heavy chain CDR1 having an amino acid sequence that is at least 80%, 85%, 90%, 91%, 92%, 93%, 94% 95%, 96%, 97%, 98%, or 99% identical to FTFSSYAMS (SEQ ID NO: 2). In one instance, an antibody or antigen-binding fragment thereof described herein comprises a heavy chain FR2 having an amino acid sequence that is at least 80%, 85%, 90%, 91%, 92%, 93%, 94% 95%, 96%, 97%, 98%, or 99% identical to WVRQAPGKGLEWV (SEQ ID NO: 3). In one instance, an antibody or antigen-binding fragment thereof described herein comprises a heavy chain CDR2 having an amino acid sequence that is at least 80%, 85%, 90%, 91%, 92%, 93%, 94% 95%, 96%, 97%, 98%, or 99% identical to SAISGSGGSTYYA (SEQ ID NO: 4). In one instance, an antibody or antigen-binding fragment thereof described herein comprises a heavy chain FR3 having an amino acid sequence that is at least 80%, 85%, 90%, 91%, 92%, 93%, 94% 95%, 96%, 97%, 98%, or 99% identical to DSVKGRFTISRDNSKNTLYLQMNSLRAEDTAVYY (SEQ ID NO: 5). In one instance, an antibody or antigen-binding fragment thereof described herein comprises a heavy chain CDR3 having an amino acid sequence that is at least 80%, 85%, 90%, 91%, 92%, 93%, 94% 95%, 96%, 97%, 98%, or 99% identical to CAKGGRDGYKGYFDYW (SEQ ID NO: 6). In one instance, an antibody or antigen-binding fragment thereof described herein comprises a heavy chain FR4 having an amino acid sequence that is at least 80%, 85%, 90%, 91%, 92%, 93%, 94% 95%, 96%, 97%, 98%, or 99% identical to GQGTLVTVSS (SEQ ID NO: 7). In other instances, an antibody or antigen-binding fragment thereof described herein comprises a heavy chain such as a heavy chain library as described in, for example, International Patent Cooperation Treaty Application No. PCT/US2018/066318, which heavy chain libraries are hereby incorporated by reference.

Provided herein are antibodies and antigen-binding fragments thereof that can bind to TfR. In some cases, such an antibody can comprise a heavy chain variable region and a light chain variable region, wherein the light chain variable region comprises (i) a complementarity determining region (CDR) 1 (CDR1) having an amino acid sequence that is at least 80%, 85%, 90%, 91%, 92%, 93%, 94% 95%, 96%, 97%, 98%, or 99% identical to any one of SEQ ID NOS: 13-27; (ii) a CDR2 having an amino acid sequence of any one of SEQ ID NOS: 32-41; and (iii) a CDR3 having an amino acid sequence that is at least 80%, 85%, 90%, 91%, 92%, 93%, 94% 95%, 96%, 97%, 98%, or 99% identical to any one of SEQ ID NOS: 47-59.

In one instance, the antibody or antigen binding fragment thereof can comprise (i) a VL CDR1 having an amino acid sequence that is at least 80%, 85%, 90%, 91%, 92%, 93%, 94% 95%, 96%, 97%, 98%, or 99% identical to RASQTLYTNYLA (SEQ ID NO: 26); KSSRSVLRTSKNKNFLA (SEQ ID NO: 27); or X₁ASX₂X₃X₄X₅X₆X₇LX₈ (SEQ ID NO: 13), wherein X₁ comprises R or Q; X₂ comprises Q or R; X₃ comprises G, D, S or N; X₄ comprises I or V; X₅ comprises S, R, G, N or K; X₆ comprises R, K, S, G, or D; X₇ comprises N, W, Y, A, R, or K; and X₈ comprises A or N; (ii) a VL CDR2 having an amino acid sequence that is at least 80%, 85%, 90%, 91%, 92%, 93%, 94% 95%, 96%, 97%, 98%, or 99% identical to X₁X₂X₃X₄X₅ X₆X₇ (SEQ ID NO: 32), wherein X₁ comprises G, A, K, W, or S; X₂ comprises A or T; X₃ comprises F or S, X₄ comprises T, R, S, or N; X₅ comprises R or L; X₆ comprises R, Q, A, or E; and X₇ comprises S, N, or T; and (iii) a VL CDR3 having an amino acid sequence that is at least 80%, 85%, 90%, 91%, 92%, 93%, 94% 95%, 96%, 97%, 98%, or 99% identical to CQX₁X₂X₃X₄X₅PX₆TF (SEQ ID NO: 47), wherein X₁ comprises Q or K; X₂ comprises S, A, G, Y, H; X₃ comprises Y, N, F, K, G, or L; X₄ comprises K, S, or R; X₅ comprises T, F, Y, A, L, R, P, or S; and X₆ comprises Y, W, F, R, L, or I.

In one instance, the antibody or antigen binding fragment thereof can comprise (i) a VL FR1 having an amino acid sequence that is at least 80%, 85%, 90%, 91%, 92%, 93%, 94% 95%, 96%, 97%, 98%, or 99% identical to X₁IX₂MTQSPX₃X₄LX₅X₆SX₇GXsRX₉TX₁₀XC (SEQ ID NO: 9), wherein X₁ comprises D or E; X₂ comprises Q or V; X₃ comprises S, D, or A; X₄ comprises S or T; X₅ comprises S or A; X₆ comprises A or V; X₇ comprises L or P; X₈ comprises D or E; X₉ comprises V or A, X₁₀ comprises I or L; and X₁₁ comprises T, N, or S; (ii) a VL CDR1 having an amino acid sequence that is at least 80%, 85%, 90%, 91%, 92%, 93%, 94% 95%, 96%, 97%, 98%, or 99% identical to RASQTLYTNYLA (SEQ ID NO: 26); KSSRSVLRTSKNKNFLA (SEQ ID NO: 27); or X₁ASX₂X₃X₄X₅X₆X₇LX₈ (SEQ ID NO: 13), wherein X₁ comprises R or Q; X₂ comprises Q or R; X₃ comprises G, D, S or N; X₄ comprises I or V; X₅ comprises S, R, G, N or K; X₆ comprises R, K, S, G, or D; X₇ comprises N, W, Y, A, R, or K; and X₈ comprises A or N; (iii) a VL FR2 having an amino acid sequence that is at least 80%, 85%, 90%, 91%, 92%, 93%, 94% 95%, 96%, 97%, 98%, or 99% identical to WYQQKPGX₁X₂PX₃LLIY (SEQ ID NO: 28), wherein X₁ comprises Q or K; X₂ comprises A or P; and X₃ comprises R or K; (iv) a VL CDR2 having an amino acid sequence that is at least 80%, 85%, 90%, 91%, 92%, 93%, 94% 95%, 96%, 97%, 98%, or 99% identical to X₁X₂X₃X₄X₅ X₆X₇ (SEQ ID NO: 32), wherein X₁ comprises G, A, K, W, or S; X₂ comprises A or T; X₃ comprises F or S, X₄ comprises T, R, S, or N; X₅ comprises R or L; X₆ comprises R, Q, A, or E; and X₇ comprises S, N, or T; (v) a VL FR3 having an amino acid sequence that is at least 80%, 85%, 90%, 91%, 92%, 93%, 94% 95%, 96%, 97%, 98%, or 99% identical to GX₁PX₂RFSGSGSGTX₃FTLTISSLQX₄EDX₅AX₆YY (SEQ ID NO: 43), wherein X₁ comprises I or V; X₂ comprises A, D, or S; X₃ comprises E or D; X₄ comprises S. P, or A; X₅ comprises F or V; and X₆ comprises V or T; (vi) a VL CDR3 having an amino acid sequence that is at least 80%, 85%, 90%, 91%, 92%, 93%, 94% 95%, 96%, 97%, 98%, or 99% identical to CQX₁X₂X₃X₄X₅PX₆TF (SEQ ID NO: 47), wherein X₁ comprises Q or K; X₂ comprises S, A, G, Y, H; X₃ comprises Y, N, F, K, G, or L; X₄ comprises K, S, or R; X₅ comprises T, F, Y, A, L, R, P, or S; and X₆ comprises Y, W, F, R, L, or I; and (vii) a VL FR4 having an amino acid sequence that is at least 80%, 85%, 90%, 91%, 92%, 93%, 94% 95%, 96%, 97%, 98%, or 99% identical to GXGTX₂X₃X₄IK (SEQ ID NO: 60), wherein X₁ comprises G, Q, or P; X₂ comprises K or R; X₃ comprises L or V; and X₄ comprises E or D.

In one instance, the antibody or antigen binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 26; (ii) a VL CDR2 having an amino acid sequence that is at least 80%, 85%, 90%, 91%, 92%, 93%, 94% 95%, 96%, 97%, 98%, or 99% identical to SEQ ID NO: 33; and (iii) a VL CDR3 having an amino acid sequence that is at least 80%, 85%, 90%, 91%, 92%, 93%, 94% 95%, 96%, 97%, 98%, or 99% identical to SEQ ID NO: 48.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 15; (ii) a VL CDR2 having an amino acid sequence that is at least 80%, 85%, 90%, 91%, 92%, 93%, 94% 95%, 96%, 97%, 98%, or 99% identical to SEQ ID NO: 34; and (iii) a VL CDR3 having an amino acid sequence that is at least 80%, 85%, 90%, 91%, 92%, 93%, 94% 95%, 96%, 97%, 98%, or 99% identical to SEQ ID NO: 49.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 14; (ii) a VL CDR2 having an amino acid sequence that is at least 80%, 85%, 90%, 91%, 92%, 93%, 94% 95%, 96%, 97%, 98%, or 99% identical to SEQ ID NO: 35; and (iii) a VL CDR3 having an amino acid sequence that is at least 80%, 85%, 90%, 91%, 92%, 93%, 94% 95%, 96%, 97%, 98%, or 99% identical to SEQ ID NO: 50.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 16; (ii) a VL CDR2 having an amino acid sequence that is at least 80%, 85%, 90%, 91%, 92%, 93%, 94% 95%, 96%, 97%, 98%, or 99% identical to SEQ ID NO: 36; and (iii) a VL CDR3 having an amino acid sequence that is at least 80%, 85%, 90%, 91%, 92%, 93%, 94% 95%, 96%, 97%, 98%, or 99% identical to SEQ ID NO: 51.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 17; (ii) a VL CDR2 having an amino acid sequence that is at least 80%, 85%, 90%, 91%, 92%, 93%, 94% 95%, 96%, 97%, 98%, or 99% identical to SEQ ID NO: 35; and (iii) a VL CDR3 having an amino acid sequence that is at least 80%, 85%, 90%, 91%, 92%, 93%, 94% 95%, 96%, 97%, 98%, or 99% identical to SEQ ID NO: 50.

Alternatively, or in addition, the antibody or antigen binding-fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 18; (ii) a VL CDR2 having an amino acid sequence that is at least 80%, 85%, 90%, 91%, 92%, 93%, 94% 95%, 96%, 97%, 98%, or 99% identical to SEQ ID NO: 37; and (iii) a VL CDR3 having an amino acid sequence that is at least 80%, 85%, 90%, 91%, 92%, 93%, 94% 95%, 96%, 97%, 98%, or 99% identical to SEQ ID NO: 50.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 19; (ii) a VL CDR2 having an amino acid sequence that is at least 80%, 85%, 90%, 91%, 92%, 93%, 94% 95%, 96%, 97%, 98%, or 99% identical to SEQ ID NO: 33; and (iii) a VL CDR3 having an amino acid sequence that is at least 80%, 85%, 90%, 91%, 92%, 93%, 94% 95%, 96%, 97%, 98%, or 99% identical to SEQ ID NO: 52.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 27; (ii) a VL CDR2 having an amino acid sequence that is at least 80%, 85%, 90%, 91%, 92%, 93%, 94% 95%, 96%, 97%, 98%, or 99% identical to SEQ ID NO: 38; and (iii) a VL CDR3 having an amino acid sequence that is at least 80%, 85%, 90%, 91%, 92%, 93%, 94% 95%, 96%, 97%, 98%, or 99% identical to SEQ ID NO: 53.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 20; (ii) a VL CDR2 having an amino acid sequence that is at least 80%, 85%, 90%, 91%, 92%, 93%, 94% 95%, 96%, 97%, 98%, or 99% identical to SEQ ID NO: 39; and (iii) a VL CDR3 having an amino acid sequence that is at least 80%, 85%, 90%, 91%, 92%, 93%, 94% 95%, 96%, 97%, 98%, or 99% identical to SEQ ID NO: 54.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 21; (ii) a VL CDR2 having an amino acid sequence that is at least 80%, 85%, 90%, 91%, 92%, 93%, 94% 95%, 96%, 97%, 98%, or 99% identical to SEQ ID NO: 40; and (iii) a VL CDR3 having an amino acid sequence that is at least 80%, 85%, 90%, 91%, 92%, 93%, 94% 95%, 96%, 97%, 98%, or 99% identical to SEQ ID NO: 55.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 22; (ii) a VL CDR2 having an amino acid sequence that is at least 80%, 85%, 90%, 91%, 92%, 93%, 94% 95%, 96%, 97%, 98%, or 99% identical to SEQ ID NO: 41; and (iii) a VL CDR3 having an amino acid sequence that is at least 80%, 85%, 90%, 91%, 92%, 93%, 94% 95%, 96%, 97%, 98%, or 99% identical to SEQ ID NO: 56.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 23; (ii) a VL CDR2 having an amino acid sequence that is at least 80%, 85%, 90%, 91%, 92%, 93%, 94% 95%, 96%, 97%, 98%, or 99% identical to SEQ ID NO: 33; and (iii) a VL CDR3 having an amino acid sequence that is at least 80%, 85%, 90%, 91%, 92%, 93%, 94% 95%, 96%, 97%, 98%, or 99% identical to SEQ ID NO: 57.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 24; (ii) a VL CDR2 having an amino acid sequence that is at least 80%, 85%, 90%, 91%, 92%, 93%, 94% 95%, 96%, 97%, 98%, or 99% identical to SEQ ID NO: 42; and (iii) a VL CDR3 having an amino acid sequence that is at least 80%, 85%, 90%, 91%, 92%, 93%, 94% 95%, 96%, 97%, 98%, or 99% identical to SEQ ID NO: 58.

Alternatively, or in addition, the antibody or antigen-binding fragment thereof can comprise (i) a VL CDR1 having an amino acid sequence that is at least 80%, 85%, 90%, 91%, 92%, 93%, 94% 95%, 96%, 97%, 98%, or 99% identical to SEQ ID NO: 25; (ii) a VL CDR2 having an amino acid sequence that is at least 80%, 85%, 90%, 91%, 92%, 93%, 94% 95%, 96%, 97%, 98%, or 99% identical to SEQ ID NO: 33; and (iii) a VL CDR3 having an amino acid sequence that is at least 80%, 85%, 90%, 91%, 92%, 93%, 94% 95%, 96%, 97%, 98%, or 99% identical to SEQ ID NO: 59.

Alternatively, or in addition, the antibody or antigen-binding fragment thereof can comprise (i) a VL FR1 having an amino acid sequence that is at least 80%, 85%, 90%, 91%, 92%, 93%, 94% 95%, 96%, 97%, 98%, or 99% identical to SEQ ID NO: 23; (ii) a VL FR2 having an amino acid sequence that is at least 80%, 85%, 90%, 91%, 92%, 93%, 94% 95%, 96%, 97%, 98%, or 99% identical to SEQ ID NO: 29; (iii) a VL FR3 having an amino acid sequence that is at least 80%, 85%, 90%, 91%, 92%, 93%, 94% 95%, 96%, 97%, 98%, or 99% identical to SEQ ID NO: 44; and (iv) a VL FR4 having an amino acid sequence that is at least 80%, 85%, 90%, 91%, 92%, 93%, 94% 95%, 96%, 97%, 98%, or 99% identical to SEQ ID NO: 61.

Alternatively, or in addition, the antibody or antigen-binding fragment thereof can comprise (i) a VL FR1 having an amino acid sequence that is at least 80%, 85%, 90%, 91%, 92%, 93%, 94% 95%, 96%, 97%, 98%, or 99% identical to SEQ ID NO: 10; (ii) a VL FR2 having an amino acid sequence that is at least 80%, 85%, 90%, 91%, 92%, 93%, 94% 95%, 96%, 97%, 98%, or 99% identical to SEQ ID NO: 30; (iii) a VL FR3 having an amino acid sequence that is at least 80%, 85%, 90%, 91%, 92%, 93%, 94% 95%, 96%, 97%, 98%, or 99% identical to SEQ ID NO: 45; and (iv) a VL FR4 having an amino acid sequence that is at least 80%, 85%, 90%, 91%, 92%, 93%, 94% 95%, 96%, 97%, 98%, or 99% identical to SEQ ID NO: 62.

Alternatively, or in addition, the antibody or antigen-binding fragment thereof can comprise (i) a VL FR1 having an amino acid sequence that is at least 80%, 85%, 90%, 91%, 92%, 93%, 94% 95%, 96%, 97%, 98%, or 99% identical to SEQ ID NO: 10; (ii) a VL FR2 having an amino acid sequence that is at least 80%, 85%, 90%, 91%, 92%, 93%, 94% 95%, 96%, 97%, 98%, or 99% identical to SEQ ID NO: 30; (iii) a VL FR3 having an amino acid sequence that is at least 80%, 85%, 90%, 91%, 92%, 93%, 94% 95%, 96%, 97%, 98%, or 99% identical to SEQ ID NO: 45; and (iv) a VL FR4 having an amino acid sequence that is at least 80%, 85%, 90%, 91%, 92%, 93%, 94% 95%, 96%, 97%, 98%, or 99% identical to SEQ ID NO: 63.

Alternatively, or in addition, the antibody or antigen-binding fragment thereof can comprise (i) a VL FR1 having an amino acid sequence that is at least 80%, 85%, 90%, 91%, 92%, 93%, 94% 95%, 96%, 97%, 98%, or 99% identical to SEQ ID NO: 12; (ii) a VL FR2 having an amino acid sequence that is at least 80%, 85%, 90%, 91%, 92%, 93%, 94% 95%, 96%, 97%, 98%, or 99% identical to SEQ ID NO: 29; (iii) a VL FR3 having an amino acid sequence that is at least 80%, 85%, 90%, 91%, 92%, 93%, 94% 95%, 96%, 97%, 98%, or 99% identical to SEQ ID NO: 44; and (iv) a VL FR4 having an amino acid sequence that is at least 80%, 85%, 90%, 91%, 92%, 93%, 94% 95%, 96%, 97%, 98%, or 99% identical to SEQ ID NO: 63.

Alternatively, or in addition, the antibody or antigen-binding fragment thereof can comprise (i) a VL FR1 having an amino acid sequence that is at least 80%, 85%, 90%, 91%, 92%, 93%, 94% 95%, 96%, 97%, 98%, or 99% identical to SEQ ID NO: 11; (ii) a VL FR2 having an amino acid sequence that is at least 80%, 85%, 90%, 91%, 92%, 93%, 94% 95%, 96%, 97%, 98%, or 99% identical to SEQ ID NO: 31; (iii) a VL FR3 having an amino acid sequence that is at least 80%, 85%, 90%, 91%, 92%, 93%, 94% 95%, 96%, 97%, 98%, or 99% identical to SEQ ID NO: 46; and (iv) a VL FR4 having an amino acid sequence that is at least 80%, 85%, 90%, 91%, 92%, 93%, 94% 95%, 96%, 97%, 98%, or 99% identical to SEQ ID NO: 63.

Alternatively, or in addition, the antibody or antigen-binding fragment thereof can comprise (i) a VL FR1 having an amino acid sequence that is at least 80%, 85%, 90%, 91%, 92%, 93%, 94% 95%, 96%, 97%, 98%, or 99% identical to SEQ ID NO: 10; (ii) a VL FR2 having an amino acid sequence that is at least 80%, 85%, 90%, 91%, 92%, 93%, 94% 95%, 96%, 97%, 98%, or 99% identical to SEQ ID NO: 30; (iii) a VL FR3 having an amino acid sequence that is at least 80%, 85%, 90%, 91%, 92%, 93%, 94% 95%, 96%, 97%, 98%, or 99% identical to SEQ ID NO: 45; and (iv) a VL FR4 having an amino acid sequence that is at least 80%, 85%, 90%, 91%, 92%, 93%, 94% 95%, 96%, 97%, 98%, or 99% identical to SEQ ID NO: 61.

Alternatively, or in addition, the antibody or antigen-binding fragment thereof can comprise (i) a VL FR1 having an amino acid sequence that is at least 80%, 85%, 90%, 91%, 92%, 93%, 94% 95%, 96%, 97%, 98%, or 99% identical to SEQ ID NO: 12; (ii) a VL FR2 having an amino acid sequence that is at least 80%, 85%, 90%, 91%, 92%, 93%, 94% 95%, 96%, 97%, 98%, or 99% identical to SEQ ID NO: 29; (iii) a VL FR3 having an amino acid sequence that is at least 80%, 85%, 90%, 91%, 92%, 93%, 94% 95%, 96%, 97%, 98%, or 99% identical to SEQ ID NO: 44; and (iv) a VL FR4 having an amino acid sequence that is at least 80%, 85%, 90%, 91%, 92%, 93%, 94% 95%, 96%, 97%, 98%, or 99% identical to SEQ ID NO: 62.

In one instance, an antibody or antigen-binding fragment thereof can comprise a VL that comprises an amino acid sequence that is at least 80%, 85%, 90%, 91%, 92%, 93%, 94%, 95%, 96%, 97%, 98% or 99% identical to an one of SEQ ID NOS: 64-65, 70-74 or 80-86.

| | SEQ ID |
|---|---|
| VL sequence | NO |
| EIVMTQSPATLSVSPGERATLSCRASQTLYTNYLAWYQQKPGQAPRLLIYG | 64 |
| ASTRATGIPARFSGSGSGTEFTLTISSLQSEDFAVYYCQQSYKTPYTFGQGTK | |
| VEIK | |
| DIQMTQSPSSLSASVGDRVTITCQASQDIIDSLNWYQQKPGKAPKLLIYAAFR | 65 |
| LRSGVPSRFSGSGSGTDFTLTISSLQPEDFATYYCQQAYSFPWTFGQGTKLEI | |
| K | |
| DIQMTQSPSSLSASVGDRVTITCRASRGISRWLAWYQQKPGKAPKLLIYAAS | 70 |
| SLQSGVPSRFSGSGSGTDFTLTISSLQPEDFATYYCQQGYSTPFTFGQGTKLEI | |
| K | |
| DIQMTQSPSSLSASVGDRVTITCRASQDIRRYLAWYQQKPGKAPKLLIYKAS | 71 |
| RLQSGVPSRFSGSGSGTDFTLTISSLQPEDFATYYCQQYNSYPRTFGQGTKLE | |
| IK | |
| DIQMTQSPSSLSASVGDRVTITCRASRGVSKWLAWYQQKPGKAPKLLIYAA | 72 |
| SSLQSGVPSRFSGSGSGTDFTLTISSLQPEDFATYYCQQGYSTPFTFGPGTKV | |
| DIK | |
| DIQMTQSPSSLSASVGDRVTITCRASRGVSSWLAWYQQKPGKAPKLLIYAA | 73 |
| STLQSGVPSRFSGSGSGTDFTLTISSLQPEDFATYYCQQGYSTPFTFGPGTKV | |
| DIK | |
| EIVMTQSPATLSVSPGERATLSCRASRSVGGALAWYQQKPGQAPRLLIYGAS | 74 |
| TRATGIPARFSGSGSGTEFTLTISSLQSEDFAVYYCQQYYSTPFTFGPGTKVDI | |
| K | |
| DIVMTQSPDSLAVSLGERATINCKSSRSVLRTSKNKNFLAWYQQKPGQPPKL | 80 |
| LIYWASTRESGVPDRFSGSGSGTDFTLTISSLQAEDVAVYYCQQYFSAPLTF | |
| GPGTKVDIK | |
| DIQMTQSPSSLSASVGDRVTITCRASQSIRRYLNWYQQKPGKAPKLLIYKAS | 81 |
| SLANGVPSRFSGSGSGTDFTLTISSLQPEDFATYYCQKYNSAPLTFGGGTKVE | |
| IK | |
| DIQMTQSPSSLSASVGDRVTITCRASQNINKNLNWYQQKPGKAPKWYKAS | 82 |
| SLESGVPSRFSGSGSGTDFTLTISSLQPEDFATYYCQQAKSLPLTFGGGTKVEI | |
| K | |
| DIQMTQSPSSLSASVGDRVTITCRASQNIGSRLNWYQQKPGKAPKWYSTS | 83 |
| NLQSGVPSRFSGSGSGTDFTLTISSLQPEDFATYYCQQYKSRPLTFGGGTKVE | |
| IK | |
| EIVMTQSPATLSVSPGERATLSCRASRSISDYLAWYQQKPGQAPRLLIYGAS | 84 |
| TRATGIPARFSGSGSGTEFTLTISSLQSEDFAVYYCQQHGSPPFTFGGGTKVEI | |
| K | |
| DIQMTQSPSSLSASVGDRVTITCRASQNIKRYLNWYQQKPGKAPKLLIYKAS | 85 |
| RLETGVPSRFSGSGSGTDFTLTISSLQPEDFATYYCQQSYSTPLTFGQGTRLEI | |
| K | |
| EIVMTQSPATLSVSPGERATLSCRASQSVRRKLAWYQQKPGQAPRLLIYGAS | 86 |
| TRATGIPARFSGSGSGTEFTLTISSLQSEDFAVYYCQQYLRSPITFGQGTRLEI | |
| K | |

In one instance, an antibody or antigen-binding fragment thereof can comprise a VH that comprises an amino acid sequence that is at least 80%, 85%, 90%, 91%, 92%, 93%, 94%, 95%, 96%, 97%, 98% or 99% identical to SEQ ID NO: 87.

| | SEQ ID |
|---|---|
| VH sequences | NO |
| EVQLLESGGGLVQPGGSLRLSCAASGFTFSSYAMSWVRQAP | 87 |
| GKGLEWVSAISGSGGSTYYADSVKGRFTISRDNSKNTLYLQ | |
| MNSLRAEDTAVYYCAKGGRDGYKGYFDYWGQGTLVTVSS | |

### Modified Antibodies

The present disclosure provides for modified antibodies. Modified antibodies can comprise antibodies which have one or more modifications which can enhance their activity, binding, specificity, selectivity, or another feature. In one aspect, the present disclosure provides for modified antibodies (which can be heteromultimers) that comprise an anti-TfR antibody as described herein. A modified antibody can be prepared using suitable techniques including, but not limited to, those described in U.S. Pat. No. 8,216,805, which methods are hereby incorporated by reference.

A modified antibody can comprise a first polypeptide and a second polypeptide. In some cases, the first polypeptide can comprise a C_{H}3 antibody constant domain. In some cases, the second polypeptide can comprise a C_{H}3 antibody constant domain. In some cases, the first polypeptide and the second polypeptide can each comprise a C_{H}3 antibody constant domain. The first and second polypeptides can meet at an engineered interface within the C_{H}3 domain.

In some cases, a modified antibody can comprise an engineered proturberance or cavity in an interface of a polypeptide. In such cases, the interface can comprise contact amino acid residues (or other non-amino acid groups such as carbohydrate groups, NADH, biotin, FAD, or heme group) in the first polypeptide which can interact with one or more “contact” amino acid residues (or other non-amino acid groups) in the interface of the second polypeptide. In some cases, the interface can be a domain of an immunoglobulin such as a variable domain or constant domain (or regions thereof). In some cases, the interface can be between the polypeptides forming a receptor to a modified antibody or antigen-binding fragment thereof or the interface between two or more ligands such as NGF, NT-3, and BDNF. The interface can comprise a CH3 domain of an immunoglobulin, which can be derived from an IgG antibody, which can be a human IgG1 antibody.

A “protuberance” refers to at least one amino acid side chain which projects from the interface of the first polypeptide and is therefore positionable in a compensatory cavity in the adjacent interface (the interface of the second polypeptide) so as to stabilize the modified antibody or antigen-binding fragment thereof, and thereby favor modified antibody or antigen-binding fragment thereof formation over homomultimer formation, for example. The protuberance may exist in the original interface or may be introduced synthetically (e.g., by altering nucleic acid encoding the interface). Normally, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The upper limit for the number of original residues which are replaced is the total number of residues in the interface of the first polypeptide. The side chain volumes of the various amino residues are shown in Table 1.

TABLE 1
Properties of Amino Acid Residues

| | | | | Accessible |
|---|---|---|---|---|
| Amino Acid | One-Letter | MASS | VOLUME | Surface Area |
| (Abbreviation) | Abbreviation | (Daltons) | (Å³) | (Å²) |
| Alanine (Ala) | A | 71.08 | 88.6 | 115 |
| Arginine (Arg) | R | 156.20 | 173.4 | 225 |
| Asparagine (Asn) | N | 114.11 | 117.7 | 160 |
| Aspartic acid (Asp) | D | 115.09 | 111.1 | 150 |
| Cysteine (Cys) | C | 103.14 | 108.5 | 135 |
| Glutamine (Gln) | Q | 128.14 | 143.9 | 180 |
| Glutamic acid (Glu) | E | 129.12 | 138.4 | 190 |
| Glycine (Gly) | G | 57.06 | 60.1 | 75 |
| Histidine (His) | H | 137.15 | 153.2 | 195 |
| Isoleucine (Ile) | I | 113.17 | 166.7 | 175 |
| Leucine (Leu) | L | 113.17 | 166.7 | 170 |
| Lysine (Lys) | K | 128.18 | 168.6 | 200 |
| Methionine (Met) | M | 131.21 | 162.9 | 185 |
| Phenylalanine (Phe) | F | 147.18 | 189.9 | 210 |
| Proline (Pro) | P | 97.12 | 122.7 | 145 |
| Serine (Ser) | S | 87.08 | 89.0 | 115 |
| Threonine (Thr) | T | 101.11 | 116.1 | 140 |
| Tryptophan (Trp) | W | 186.21 | 227.8 | 255 |
| Tyrosine (Tyr) | Y | 163.18 | 193.6 | 230 |
| Valine (Val) | V | 99.14 | 140.0 | 155 |

Import residues for the formation of a protuberance can be naturally-occurring amino acid residues, which can be selected from arginine (R), phenylalanine (F), tyrosine (Y) and tryptophan (W). In some cases, the original residue for the formation of the protuberance has a small side chain volume, such as alanine, asparagine, aspartic acid, glycine, serine, threonine, or valine.

A cavity can be at least one amino acid side chain, which can be recessed from the interface of a second polypeptide, and which can accommodate a corresponding protuberance on an adjacent interface of a first polypeptide. In some cases, a cavity can be recessed from the interface of a first polypeptide, which can accommodate a corresponding protuberance on an adjacent interface of a second polypeptide. The cavity may exist in the original interface or may be introduced synthetically (e.g., by altering one or more nucleic acids encoding the interface). To achieve this, the nucleic acid encoding at least one “original” amino acid residue in the interface of a polypeptide can be replaced with DNA encoding at least one “import” amino acid residue, which can have a smaller side chain volume than the original amino acid residue. In some cases, there can be more than one original residue or more than one corresponding import residue. The upper limit for the number of original residues which are replaced can be the total number of residues in the interface of a polypeptide. Import residues for the formation of a cavity can be naturally-occurring amino acid residues which can be selected from alanine (A), serine (S), threonine (T), and valine (V). In some cases, the original residue for the formation of the protuberance has a large side chain volume, such as tyrosine, arginine, phenylalanine, or tryptophan.

An original amino acid residue can be one which is replaced by an import residue, which can have a smaller or larger side chain volume than the original residue. The import amino acid residue can be a naturally occurring or non-naturally occurring amino acid residue. Naturally occurring amino acid residues can be encoded by the genetic code and can be the amino acid residues listed in Table 1. A non-naturally occurring amino acid residue can be a residue which is not encoded by the genetic code, but which can covalently bind adjacent amino acid residue(s) in the polypeptide chain. Examples of non-naturally occurring amino acid residues can include norleucine, ornithine, norvaline, homoserine, and other amino acid residue analogues.

A protuberance can be positionable in the cavity if the spatial location of the protuberance and cavity on the interface of the first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe, and Trp may not typically extend perpendicularly from the axis of the interface and can have preferred conformations, the alignment of a protuberance with a corresponding cavity can rely on modeling the protuberance/cavity pair based upon a three-dimensional structure, such as a structure obtained by X-ray crystallography or nuclear magnetic resonance (NMR).

An original nucleic acid can be the nucleic acid encoding a polypeptide of interest which can be altered (i.e., genetically engineered or mutated) to encode a feature such as a protuberance or cavity. The original or starting nucleic acid can be a naturally occurring nucleic acid or it can comprise a nucleic acid which can have been previously altered (e.g., a humanized antibody fragment). Altering a nucleic acid can be achieved, for example, by inserting, deleting or replacing at least one codon encoding an amino acid residue of interest. Normally, a codon encoding an original residue can be replaced by a codon encoding an import residue.

A protuberance or cavity can be introduced into the interface of the first or second polypeptide by synthetic means, such as by recombinant techniques, in vitro peptide synthesis, other acceptable techniques for introducing non-naturally-occurring amino acid residues, by enzymatic or chemical coupling of peptides, or some combination of these techniques. A protuberance or cavity which is introduced can be non-naturally occurring or non-native, such that it may not exist in nature or in the original polypeptide.

The import amino acid residue for forming the protuberance can have a relatively small number of rotamers (e.g., about 3-6). A rotomer can be an energetically favorable conformation of an amino acid side chain.

In some cases, a modified antibody having a first polypeptide can comprise an engineered protuberance in the interface of the first polypeptide. In some cases, a modified antibody having a second polypeptide can comprise an engineered protuberance in the interface of the second polypeptide.

In some cases, a modified antibody having a first polypeptide can comprise an engineered cavity in the interface of the first polypeptide. In some cases, a modified antibody having a second polypeptide can comprise an engineered cavity in the interface of the second polypeptide.

A modified antibody having a first polypeptide and a second polypeptide comprising an engineered protuberance in the interface of the first polypeptide can have an engineered cavity in the interface of the second polypeptide. In some cases, a modified antibody having a first polypeptide and a second polypeptide comprising an engineered protuberance in the interface of the second polypeptide can have an engineered cavity in the interface of the first polypeptide.

An engineered protuberance in the interface of a first polypeptide can be within its C_{H}3 domain, which can be created by replacing at least one contact residue of the first polypeptide within its C_{H}3 domain, and wherein the second polypeptide can comprise an engineered cavity in the interface of the second polypeptide within its C_{H}3. In some cases, an engineered protuberance in the interface of a second polypeptide can be within its C_{H}3 domain, which can be created by replacing at least one contact residue of the first polypeptide within its C_{H}3 domain, and wherein the first polypeptide can comprise an engineered cavity in the interface of the second polypeptide within its C_{H}3.

A modified antibody can comprise, for example, a bispecific antibody, a bispecific immunoadhesion, or an antibody/immunoadhesion chimera.

In one instance, a modified antibody can comprise a first polypeptide, which can each comprise a C_{H}3 antibody constant domain, wherein the first and second polypeptides can meet at an engineered interface within the C_{H}3 domain, and wherein the first polypeptide or the second polypeptide can comprise a C_{H}3 domain which can be encoded by the nucleic acid sequence of SEQ ID NO: 8, or by a nucleic acid sequence that is at least 80%, 85%, 90%, 91%, 92%, 93%, 94% 95%, 96%, 97%, 98%, or 99% identical to SEQ ID NO: 8. In another instance, a modified antibody can comprise a first polypeptide, which can each comprise a C_{H}3 antibody constant domain, wherein the first and second polypeptides can meet at an engineered interface within the C_{H}3 domain, and wherein the first polypeptide or the second polypeptide can comprise a C_{H}3 domain of SEQ ID NO: 6, or a sequence that is at least 80%, 85%, 90%, 91%, 92%, 93%, 94% 95%, 96%, 97%, 98%, or 99% identical to SEQ ID NO: 6.

The first polypeptide of a modified antibody herein can comprise an engineered proturberance in the interface of the first polypeptide within its C_{H}3 domain. Such a protuberance can be created by replacing at least one contact residue of the first polypeptide within its C_{H}3 domain. Additionally, the second polypeptide can comprise an engineered cavity in the interface of the second polypeptide within its C_{H}3 domain.

In some cases, the engineered protuberance in the interface of the first polypeptide can be positional in the engineered cavity of the second polypeptide so as to form a protuberance-into-cavity mutant pair. In such a mutant pair, an engineered protuberance can “fit” into a cavity. In such cases, the structure of the protuberance can be a same size and shape as the structure of the cavity. In some cases, the structure of the protuberance can be larger, or can accommodate the cavity with a small amount of extra space. In some cases, the shape of a protuberance, a cavity, or both can dictate the positioning, spacing, or orientation of the two polypeptides with respect to each other. In some cases, the engineered interface within the CH3 domain comprises at least two protuberance-into-cavity mutant pairs.

In some cases, the at least two protuberance-into-cavity mutant pairs are created by creating at least one protuberance and at least one cavity on the first polypeptide and creating at least one cavity and at least one protuberance on the second polypeptide.

In some cases, the at least two protuberance-into-cavity mutant pairs are created by creating more than one protuberance on the first polypeptide and creating more than one cavity on the second polypeptide.

A modified antibody can comprise a bispecific modified antibody, a trispecific modified antibody or antigen-binding fragment thereof, or a tetraspecific modified antibody or antigen-binding fragment thereof. A bispecific modified antibody can be able to specifically bind to 2 targets. In some cases, one of the targets a bispecific modified antibody can specifically bind to can be a TfR. A trispecific modified antibody can be able to specifically bind to 3 targets. In some cases, one of the targets a trispecific modified antibody can specifically bind to can be a TfR. A tetraspecific modified antibody can be able to specifically bind to 4 targets. In some cases, one of the targets a tetraspecific modified antibody can specifically bind to can be a TfR.

In some cases, a modified antibody can comprise an isolated modified antibody or a purified modified antibody. An isolated or purified modified antibody can be a modified antibody which has been identified and separated and/or recovered from a component of its natural cell culture environment. Contaminant components of its natural environment can be materials which can interfere with diagnostic or therapeutic uses for the modified antibody or antigen-binding fragment thereof, and can include enzymes, hormones, or other proteinaceous or non-proteinaceous solutes. A modified antibody can be purified (1) to greater than 95% by weight of protein as determined, for example, by the Lowry method, or in some cases to greater than 99% by weight of protein as determined, for example, by the Lowry method, (2) to a degree sufficient to obtain at least 15 residues of N-terminal or internal amino acid sequence by use of a spinning cup sequenator, and/or (3) to homogeneity by SDS-PAGE under reducing or non-reducing conditions using, for example, Coomassie blue or silver stain.

A modified antibody can comprise a human modified antibody. Also included herein are amino acid sequence variants of the modified antibody which can be prepared by introducing appropriate nucleotide changes into the modified antibody DNA, or by synthesis of the desired modified antibody polypeptide. Such variants include, for example, deletions from, or insertions or substitutions of, residues within the amino acid sequences of the first and second polypeptides forming the modified antibody. Any combination of deletion, insertion, and substitution is made to arrive at the final construct, provided that the final construct possesses the desired antigen-binding characteristics. The amino acid changes also may alter post-translational processes of the modified antibody, such as changing the number or position of glycosylation sites.

“Alanine scanning mutagenesis” can be a useful method for identification of certain residues or regions of the modified antibody polypeptides that might be preferred locations for mutagenesis.

Here, a residue or group of target residues are identified (e.g., charged residues such as Arg, Asp, His, Lys, and Glu) and replaced by a neutral or negatively charged amino acid (for example, alanine or polyalanine) to affect the interaction of the amino acids with the surrounding aqueous environment in or outside the cell. Those domains demonstrating functional sensitivity to the substitutions then are refined by introducing further or other variants at or for the sites of substitution. Thus, while the site for introducing an amino acid sequence variation is predetermined, the nature of the mutation per se need not be predetermined.

Normally the mutations can involve conservative amino acid replacements in non-functional regions of the modified antibody. Exemplary mutations are shown in Table 2 below.

TABLE 2

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Covalent modifications of antibody, antigen binding fragment, or modified antibody polypeptides are included within the scope of this disclosure. Covalent modifications of the modified antibody can be introduced into the molecule by reacting targeted amino acid residues of the modified antibody or fragments thereof with an organic derivatizing agent that can be capable of reacting with selected side chains or the N- or C-terminal residues. Another type of covalent modification of the modified antibody polypeptide can comprise altering the native glycosylation pattern of the polypeptide. Herein, “altering” can mean deleting one or more carbohydrate moieties found in the original modified antibody, and/or adding one or more glycosylation sites that are not present in the original modified antibody. Addition of glycosylation sites to the modified antibody polypeptide can be accomplished by altering the amino acid sequence such that it contains one or more N-linked glycosylation sites. The alteration may also be made by the addition of, or substitution by, one or more serine or threonine residues to the original modified antibody sequence (for O-linked glycosylation sites). For ease, the modified antibody amino acid sequence can be altered through changes at the DNA level, particularly by mutating the DNA encoding the modified antibody polypeptide at preselected bases such that codons are generated that will translate into the desired amino acids. Another means of increasing the number of carbohydrate moieties on the modified antibody polypeptide is by chemical or enzymatic coupling of glycosides to the polypeptide. Removal of carbohydrate moieties present on the modified antibody can be accomplished chemically or enzymatically.

Another type of covalent modification of modified antibody comprises linking the modified antibody polypeptide to one of a variety of non-proteinaceous polymers, e.g., polyethylene glycol, polypropylene glycol, or polyoxyalkylenes.

Since it is often difficult to predict in advance the characteristics of a variant modified antibody, it will be appreciated that some screening of the recovered variants may be needed to select an optimal variant.

Methods for complexing binding agents or the antibody or antigen-binding fragments thereof described herein with another agent have been described (e.g., antibody conjugates as reviewed by Ghetie et al., 1994, Pharmacol. Ther. 63:209-34). Such methods may utilize one of several available heterobifunctional reagents used for coupling or linking molecules. Additional radionuclides are further described herein along with additional methods for linking molecules, such as therapeutic and diagnostic labels.

Antibodies can be modified for various purposes such as, for example, by addition of polyethylene glycol (PEG). PEG modification (PEGylation) can lead to one or more of improved circulation time, improved solubility, improved resistance to proteolysis, reduced antigenicity and immunogenicity, improved bioavailability, reduced toxicity, improved stability, and easier formulation.

In one instance, Fc portions of antibodies can be modified to increase half-life of the molecule in the circulation in blood when administered to a subject. Modifications can be determined using suitable means such as, for example, those described in U.S. Pat. No. 7,217,798, which is hereby incorporated by reference in its entirety.

Other methods of improving the half-life of antibody-based fusion proteins in circulation are also described, for example, in U.S. Pat. Nos. 7,091,321 and 6,737,056, each of which is hereby incorporated by reference. Additionally, antibodies may be produced or expressed so that they do not contain fucose on their complex N-glycoside-linked sugar chains. The removal of the fucose from the complex N-glycoside-linked sugar chains is known to increase effector functions of the antibodies and antigen-binding fragments including, but not limited to, antibody dependent cell-mediated cytotoxicity (ADCC) and complement dependent cytotoxicity (CDC). Similarly, antibodies can be attached at their C-terminal end to all or part of an immunoglobulin heavy chain derived from any antibody isotype, e.g., IgG, IgA, IgE, IgD, and IgM and any of the isotype sub-classes, e.g., IgG1, IgG2b, IgG2a, IgG3, and IgG4.

Additionally, the antibodies described herein can also be modified so that they are able to cross the blood-brain barrier. Such modification of the antibodies described herein allows for the treatment of brain diseases. Exemplary modifications to allow proteins such as antibodies to cross the blood-brain barrier are described in US Patent Publication 20070082380 A1 which is hereby incorporated by reference with respect to such modifications.

Glycosylation of immunoglobulins has been shown to have significant effects on their effector functions, structural stability, and rate of secretion from antibody-producing cells. The carbohydrate groups responsible for these properties are generally attached to the constant (C) regions of the antibodies. For example, glycosylation of IgG at asparagine 297 in the C_{H} 2 domain is required for full capacity of IgG to activate the classical pathway of complement-dependent cytolysis. Glycosylation of IgM at asparagine 402 in the C_{H} 3 domain may be needed in some instances for proper assembly and cytolytic activity of an antibody. Additionally, antibodies may be produced or expressed so that they do not contain fucose on their complex N-glycoside-linked sugar chains. The removal of the fucose from the complex N-glycoside-linked sugar chains is known to increase effector functions of the antibodies and antigen-binding fragments including, but not limited to, antibody dependent cell-mediated cytotoxicity (ADCC) and complement dependent cytotoxicity (CDC). These “defucosylated” antibodies may be produced through a variety of systems utilizing molecular cloning techniques including, but not limited to, transgenic animals, transgenic plants, or cell-lines that have been genetically engineered so that they no longer contain the enzymes and biochemical pathways necessary for the inclusion of a fucose in the complex N-glycoside-linked sugar chains (also known as fucosyltransferase knock-out animals, plants, or cells). Non-limiting examples of cells that can be engineered to be fucosyltransferase knock-out cells include CHO cells, SP2/0 cells, NS0 cells, and YB2/0 cells.

Glycosylation at a variable domain framework residue can alter the binding interaction of the antibody with antigen. The present disclosure includes criteria by which a limited number of amino acids in the framework or CDRs of an immunoglobulin chain can be chosen to be mutated (e.g., by substitution, deletion, and/or addition of residues) in order to increase the affinity of an antibody. Glycosylation of antibodies is further described in U.S. Pat. No. 6,350,861, which is incorporated by reference herein with respect to glycosylation.

Antibodies, or antigen-binding fragments thereof, described herein can also be used as immunoconjugates. As used herein, for purposes of the specification and claims, immunoconjugates refer to conjugates comprised of the anti-TfR antibodies or fragments thereof according to the present disclosure and at least one therapeutic label. Therapeutic labels include antitumor agents and angiogenesis-inhibitors. Such antitumor agents include, but are not limited to, toxins, drugs, enzymes, cytokines, radionuclides, and photodynamic agents. Toxins include, but are not limited to, ricin A chain, mutant Pseudomonas exotoxins, diphtheria toxoid, streptonigrin, boamycin, saporin, gelonin, and pokeweed antiviral protein. Drugs include, but are not limited to, daunorubicin, methotrexate, and calicheamicin. Radionuclides include radiometals. Non-limiting examples of radiometals include, for example, ³², ³³P, ⁴³K, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/⁸¹MKr, ⁸⁷MSr, ⁹⁰Y ⁹⁷Ru, ⁹⁹Tc, ¹⁰⁰Pd, ¹⁰¹Rh, ¹⁰³Pb, ¹⁰⁵R, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi, and ²¹³Bi. Cytokines include, but are not limited to, transforming growth factor beta (TGF-β), interleukins, interferons, and tumor necrosis factors. Photodynamic agents include, but are not limited to, porphyrins and their derivatives. Additional therapeutic labels are also contemplated herein. The methods for complexing the anti-TfR mAbs or antigen-binding fragments thereof with at least one agent have been described (i.e., antibody conjugates as reviewed by Ghetie et al., 1994, Pharmacol. Ther. 63:209-34). Such methods may utilize one of several available heterobifunctional reagents used for coupling or linking molecules. Linkers for conjugating antibodies to other moieties are within the scope of the present disclosure.

Associations (binding) between antibodies and labels include, but are not limited to, covalent and non-covalent interactions, chemical conjugation, as well as recombinant techniques. Other linkers have been described herein.

Antibodies, or antigen-binding fragments thereof, can be modified for various purposes such as, for example, by addition of polyethylene glycol (PEG). PEG modification (PEGylation) can lead to one or more of improved circulation time, improved solubility, improved resistance to proteolysis, reduced antigenicity and immunogenicity, improved bioavailability, reduced toxicity, improved stability, and easier formulation (for a review, see, Francis et al., International Journal of Hematology 68:1-18, 1998).

Additionally, binding agents, antibodies, and antigen-binding fragments thereof may be produced or expressed so that they do not contain fucose on their complex N-glycoside-linked sugar chains. The removal of the fucose from the complex N-glycoside-linked sugar chains is known to increase effector functions of the antibodies and antigen-binding fragments including, but not limited to, antibody dependent cell-mediated cytotoxicity (ADCC) and complement dependent cytotoxicity (CDC). Similarly, antibodies or antigen-binding fragments thereof that can bind TfR can be attached at their C-terminal end to all or part of an immunoglobulin heavy chain derived from any antibody isotype, e.g., IgG, IgA, IgE, IgD, and IgM and any of the isotype sub-classes, e.g., IgG1, IgG2b, IgG2a, IgG3, and IgG4.

An antibody or antigen-binding fragment thereof can be conjugated to, or recombinantly engineered with, an affinity tag (e.g., a purification tag). Affinity tags such as, for example, His6 tags (His-His-His-His-His-His; SEQ ID NO: 112) have been described.

### Barrier Crossing and Multivalent Agents

The blood-brain barrier is a barrier that can be a highly selective boarder separating circulating blood from the brain, central nervous system, and cerebrospinal fluid. The blood-brain barrier can be a semi-permeable, membranous barrier, and can be at the interface between blood and cerebral tissue. The blood-brain barrier can control exchange of molecules between blood and cerebral tissue, for example, by controlling the diffusion, facilitated diffusion, passive transport, or active transport of molecules across the blood-brain barrier. Such control can make it difficult to transport therapeutic agents or diagnostic agents into and/or across the blood-brain barrier.

To facilitate the crossing of a therapeutic agent or diagnostic agent across the blood-brain barrier, transporters naturally expressed in the blood-brain barrier can be exploited. One such transporter, TfR, is expressed at the blood-brain barrier, and can help to facilitate entry into the brain for molecules which are able to bind to TfR.

Transferrin and TfR at the blood-brain barrier can mediate endocytosis as a means of bringing iron into the brain. Transferrin-mediated endocytosis can aid in the passage of antibodies and antigen-binding fragments thereof, as well as modified antibodies or antigen-binding fragment thereof disclosed herein across the blood-brain barrier.

In some cases, an antibody or antigen-binding fragment thereof, or a modified antibody or antigen-binding fragment thereof can be capable of crossing the blood-brain barrier. In some cases, the antibody or antigen-binding fragment can cross the blood-brain barrier in one direction only (e.g., in only). In some cases, the antibody or antigen-binding fragment can cross the blood-brain barrier in two directions (e.g., in and out).

Antibodies and antigen-binding fragments herein can cross other blood-tissue barriers in addition to or instead of the blood-brain barrier. Such blood-tissue barriers can provide a barrier between a tissue or organ and the blood. Blood-tissue barriers can comprise tight junctions, epithelial cells, transport proteins, and other features. Blood-tissue barriers can make the transport of some drugs to their target difficult, particularly if the drug is unable to cross a blood-tissue barrier well or at all. Other barriers can include the blood-cerebrospinal fluid (CSF) barrier (choroid plexus), the blood-testis barrier (Sertoli cells), the placenta (maternofetal interface), the blood-retina barrier (retinal pigment epithelium), or the blood-thymus barrier (epithelial reticular cells). Such antibodies or antigen-binding fragments can provide a therapeutic effect or deliver another therapeutic agent to a tissue or tissues via one or more blood-tissue barriers they are able to cross.

In some cases, an antibody or antigen-binding fragment which is bispecific or multispecific can have affinity to a blood-tissue barrier surface molecule which is not TfR which can allow the antibody to home to a specific blood-tissue barrier for TfR-mediated endocytosis. In such cases, the antibody or antibody fragment can be preferentially transported across a selected blood-tissue barrier. For example, some antibodies and antibody fragments can be preferentially transported across the blood-brain barrier, the blood-CSF barrier, the blood-testis barrier, the placenta, the blood-retina barrier, the blood-thymus barrier, or a combination thereof. Such homing can affect the therapeutic dose, the administered dose, the volume of distribution, metabolism, side effects, or other features or effects of an antibody, antigen-binding fragment, or associated therapeutic agent. In some cases, for example, side effects can be reduced by such homing if less therapeutic agent is administered or the distribution of the therapeutic agent is limited.

In some cases, an antibody or antigen-binding fragment can carry a cargo across a blood-tissue barrier. Such a cargo can be a therapeutic agent, for example. In some cases, an antibody or antigen-binding fragment can carry more than one therapeutic agent across a blood-tissue barrier.

Such a therapeutic agent may be unable to cross the barrier without assistance or may be unable to efficiently cross the barrier without assistance. In some cases, a therapeutic agent can cross a barrier at least about 2, 3, 4, 5, 10, 15, 20, 25, 30, 35, 40, 45, 50, 100, 200, 300, 400, 500, 600, 700, 800, 900, or 1000 times faster when carried by an antibody or antigen-binding fragment as described herein. In some cases, a therapeutic agent can cross a barrier at least about 2, 3, 4, 5, 10, 15, 20, 25, 30, 35, 40, 45, 50, 100, 200, 300, 400, 500, 600, 700, 800, 900, or 1000 times more efficiently when carried by an antibody or antigen-binding fragment as described herein.

This disclosure provides for binding agents. A binding agent can comprise an antibody or an antigen-binding fragment thereof, or a modified antibody or antigen-binding fragment thereof, as described herein, and can further comprise a fusion protein. In some cases, a binding agent can additionally comprise a linker.

A fusion protein can be a protein which can be made of parts which can be from different sources. In some cases, a fusion peptide can be a protein which can be made of 2 or more different or parts of proteins which can be different or from different sources. In some cases, a fusion protein can comprise 2, 3, 4, 5, 6, or more parts. The parts of a fusion protein can be connected, for example, by a linker.

In some cases, a fusion protein can be created through the joining of two or more genes, where the two or more genes can have originally coded for separate proteins or proteins from separate sources.

Parts of a fusion protein can be connected using a linker. A linker can provide spacing between domains, support correct protein folding, permit an important domain interaction, reinforce stability, or reduce steric hindrance. A linker can be a flexible linker, which can comprise small glycine residues. A flexible linker can provide the ability to take a dynamic or adaptable shape. A linker can be a rigid linker, which can comprise cyclic residues such as proline residues. A rigid linker can provide or maintain a specific spacing between domains. A linker can be a cleavable linker. A cleavable linker can allow the release of one or more fused domains under certain reaction conditions. Such reaction conditions can be a specific pH gradient or contact with another biomolecule.

In some cases, a fusion protein can comprise another protein bond to the C-terminal side of the binding agent. The other protein can be a lysosomal enzyme, which can be human iduronate 2-sulfatase, another antibody or antigen-binding fragment, a signaling molecule which is a protein, a protein sequence which can be a homing sequence, a protein which can bind to a cell surface molecule (e.g., for homing), or another protein.

In some cases, a binding agent can further comprise a linker, which can comprise a peptide sequence. Such a linker can allow the genetic fusion of two or more proteins. In some cases, the resulting fusion protein can comprise features or a combination of features not present in an individual component of the fusion protein. A linker can be a flexible linker or a rigid linker. In some cases, a linker can be a cleavable linker.

In some cases, the linker can comprise a linker sequence consisting of 3-50 amino acids. A linker sequence can comprise at least 3, 5, 10, 15, 20, 25, 30, 35, 40, 45, or 50 amino acids. In some cases, a linker sequence can comprise at most 3, 5, 10, 15, 20, 25, 30, 35, 40, 45, or 50 amino acids. In some cases, a linker sequence can comprise about 3, 5, 10, 15, 20, 25, 30, 35, 40, 45, or 50 amino acids. In some cases, a linker sequence can comprise between 3 and 50, between 3 and 45, between 3 and 40, between 3 and 35, between 3 and 30, between 3 and 25, between 3 and 20, between 3 and 15, between 3 and 10, between 3 and 5, between 5 and 50, between 5 and 45, between 5 and 40, between 5 and 35, between 5 and 30, between 5 and 25, between 5 and 20, between 5 and 15, between 5 and 10, between 10 and 50, between 10 and 45, between 10 and 40, between 10 and 35, between 10 and 30, between 10 and 25, between 10 and 20, between 10 and 15, between 15 and 50, between 15 and 45, between 15 and 40, between 15 and 35, between 15 and 30, between 15 and 25, between 15 and 20, between 20 and 50, between 20 and 45, between 20 and 40, between 20 and 35, between 20 and 30, between 20 and 25, between 25 and 50, between 25 and 45, between 25 and 40, between 25 and 35, between 25 and 30, between 30 and 50, between 30 and 45, between 30 and 40, between 30 and 35, between 35 and 50, between 35 and 45, between 35 and 40, between 40 and 50, between 40 and 45, or between 45 and 50 amino acids.

In some cases, the linker can form a link between a binding agent and the other protein. Such a link can be permanent, or can be cleavable, for example, when a particular enzyme, pH, or other condition is present.

In some cases, the other protein can be useful for bringing the binding agent into contact with a central nervous system (CNS) of a subject. For example, the other protein can have affinity to another protein on the blood-brain barrier, to allow the molecule to home to the brain rather than another tissue or barrier expressing TfR. In some cases, the other protein can have affinity to a protein in the brain tissue or CNS so that it can home to a specific region or cell type in the brain or CNS once inside.

In some cases, a binding agent can comprise one or more antigen-binding sites that specifically bind to one or more brain antigens. A brain antigen that is to be targeted by a binding agent described herein includes, but is not limited to, beta-secretase 1 (BACE1), Abeta, epidermal growth factor receptor (EGFR), human epidermal growth factor receptor 2 (HER2), tau, apolipoprotein E (ApoE), alpha-synuclein, CD20, huntingtin, prion protein (PrP), CD19, GABA family members (e.g., GABA), leucine rich repeat kinase 2(LRRK2), parkin, presenilin 1, presenilin 2, gamma secretase, death receptor 6 (DR6), amyloid precursor protein (APP), p75 neurotrophin receptor (p75NTR), caspase 6, Tropomyosin receptor kinase A (TRKA), Tropomyosin receptor kinase B (TRKB), Tropomyosin receptor kinase C (TRKC), α synucleins, β synucleins, gamma synucleins, Tau, vascular endothelial growth factor (VEGF), neuropilin, a Semaphorin (e.g., Semaphorin 3A, Semaphorin 4A, or Semaphorin 6A), myelin basic protein (MBP), myelin oligodendrocyte glycoprotein (MOG), proteolipid protein (PLP), MAG, aquaporin 4, glutamate receptor, or a combination thereof.

In one case, a binding agent comprises an antigen-binding site that selectively binds to TfR and an antigen-binding site that selectively binds to beta-secretase 1 (BACE1). Examples of agents that comprise an antigen-binding site that selectively binds to beta-secretase 1 (BACE1) include, but are not limited to, monoclonal antibodies and commercial antibodies, such as PA1-757 and MA1-177 from ThermoFisher Scientific; and ab183612, ab2077, and ab108394 from Abcam.

In one case, a binding agent comprises an antigen-binding site that selectively binds to TfR and an antigen-binding site that selectively binds to CD19. Examples of agents that comprise an antigen-binding site that selectively binds to CD19 include, but are not limited to, monoclonal antibodies and commercial antibodies, such as TECARTUS™, MDX-1342, Loncastuximab tesirine, HIB19, REA675, TA506236, etc.

In one case, a binding agent comprises an antigen-binding site that selectively binds to TfR and an antigen-binding site that selectively binds to GABA. Examples of agents that comprise an antigen-binding site that selectively binds to GABA include, but are not limited to, monoclonal antibodies and commercial antibodies, such as LS-C295838 and LS-C708643 from LS Bio; and monoclonal [EPR23539-255] (ab252430) from Abcam.

In one case, a binding agent comprises an antigen-binding site that selectively binds to TfR and an antigen-binding site that selectively binds to Amyloid beta (Abeta). Examples of agents that comprise an antigen-binding site that selectively binds to Abeta include, but are not limited to, 0-trace/prostaglandin D2 synthase (β-trace), transthyretin (TTR), cystatin C (CysC), and al-antitrypsin (AAT), as well as monoclonal antibodies and commercial antibodies, such as MAB96182, MAB9618, and MAB96181 from R&D Systems; ab2539 from Abcam; and SIG-39320 from BioLegend.

In one case, a binding agent comprises an antigen-binding site that selectively binds to TfR and an antigen-binding site that selectively binds to EGFR. Examples of agents that comprise an antigen-binding site that selectively binds to EGFR include, but are not limited to, heparin-binding EGF-like growth factor (HB-EGF), transforming growth factor-α (TGF-α), amphiregulin (AR), epiregulin (EPR), epigen betacellulin (BTC), neuregulin-1 (NRG1), neuregulin-2 (NRG2), neuregulin-3 (NRG3), and neuregulin-4 (NRG4), as well as monoclonal antibodies and commercial antibodies, such as PA1-1110 and MA5-13269 from ThermoFisher Scientific; and ab52894 from Abcam.

In one case, a binding agent comprises an antigen-binding site that selectively binds to TfR and an antigen-binding site that selectively binds to HER2. Examples of agents that comprise an antigen-binding site that selectively binds to HER2 include, but are not limited to, heparin-binding EGF-like growth factor (HB-EGF), transforming growth factor-α (TGF-α), amphiregulin (AR), epiregulin (EPR), epigen betacellulin (BTC), neuregulin-1 (NRG1), neuregulin-2 (NRG2), neuregulin-3 (NRG3), and neuregulin-4 (NRG4), as well as monoclonal antibodies and commercial antibodies, such as CA5-14057, MA5-13105, and MA1-35720 from ThermoFisher Scientific; 10004-R205, 10004-R511-F, and 10004-R511-P from Sino Biological; and MAB1129 from R&D Systems.

In one case, a binding agent comprises an antigen-binding site that selectively binds to TfR and an antigen-binding site that selectively binds to tau. Examples of agents that comprise an antigen-binding site that selectively binds to tau include, but are not limited to, alpha-synuclein, FYN, S100B, and 14-3-3 protein zeta/delta (YWHAZ), as well as monoclonal antibodies and commercial antibodies, such as GTX100866 from GeneTex; MA1020 and AHB0042 from ThermoFisher Scientific; and AF3494, MAB3494, and MAB34941 from R&D Systems.

In one case, a binding agent comprises an antigen-binding site that selectively binds to TfR and an antigen-binding site that selectively binds to apolipoprotein E (ApoE). Examples of agents that comprise an antigen-binding site that selectively binds to ApoE include, but are not limited to, ApoE receptor or a fragment thereof, including LDLRs, Apoer2, very low-density lipoprotein receptors (VLDLRs), and lipoprotein receptor-related protein 1 (LRP1), as well as monoclonal antibodies and commercial antibodies, such as GTX129086 from GeneTex; PA5-82802, MA1-80192, and MA1-27189 from ThermoFisher Scientific; and ab92544 from Abcam.

In one case, a binding agent comprises an antigen-binding site that selectively binds to TfR and an antigen-binding site that selectively binds to alpha synuclein. Examples of agents that comprise an antigen-binding site that selectively binds to alpha synuclein include, but are not limited to, synaptobrevin-2, cytochrome b-cl complex subunit 2, stomatin protein 2, GABA aminotransferase, fumarylacetoacetate hydrolase domain-containing protein 2, myelin proteolipid protein, Abl interactor 1, phosphatidylethanolamine-binding protein 1, TNF receptor-associated protein 1, tropomodulin-2, and v-type proton ATPase subunit F; as well as anti-synuclein monoclonal antibodies Syn211, 4B12, 14H2L11, and Syn505 from Thermo Fisher Scientific.

In one case, a binding agent comprises an antigen-binding site that selectively binds to TfR and an antigen-binding site that selectively binds to CD20. Examples of agents that comprise an antigen-binding site that selectively binds to CD20 include, but are not limited to, obinutuzumab, rituximab, ocaratuzumab, ocrelizumab, TRU-015, and veltuzumab.

In one case, a binding agent comprises an antigen-binding site that selectively binds to TfR and an antigen-binding site that selectively binds to huntingtin. Examples of agents that comprise an antigen-binding site that selectively binds to huntingtin include, but are not limited to, a-adaptin, Akt/PKB, CBP, CA150, CIP4, CtBP, FIP2, Grb2 HAP1, HAP40, HIP1, HIP14/HYP-H, N-CoR, NF-kb, p53, PACSIN1, PSD-95, RasGAP, sh3gl3, sin3a, and Sp1, as well as monoclonal antibodies and commercially available antibodies, such as GTX132433 from GeneTex; ab109115, ab45169, and ab225573 from Abcam; and PA5-53068, MA5-26232, MA1-46412, and MA1-82100 from Thermo Fisher Scientific.

In one case, a binding agent comprises an antigen-binding site that selectively binds to TfR and an antigen-binding site that selectively binds to prion protein (PrP). Examples of agents that comprise an antigen-binding site that selectively binds to PrP include, but are not limited to, monoclonal antibodies and commercially available antibodies, such as GTX101063 from GeneTex; ab52604, ab61409, ab3531, ab703, ab238428, ab6664, ab14219, and ab22256 from Abcam; and MA1-750, 14-9230-82, 12-9230-42, PA5-27313, MA1-10152, PA1-84495, and MA1-82828 from ThermoFisher Scientific.

In one case, a binding agent comprises an antigen-binding site that selectively binds to TfR and an antigen-binding site that selectively binds to LRRK2. Examples of agents that comprise an antigen-binding site that selectively binds to LRRK2 include, but are not limited to, PRICKLE1, CELSR1, FLOTILLIN-2 and CULLIN-3, as well as monoclonal antibodies and commercially available antibodies, such as PA1-16770, PA5-18319, PA5-13872, PA5-13874, MA5-11154, and MA5-11155 from ThermoFisher Scientific; GTX113067 from GeneTex; AF6674 and MAB6674 from R&D Systems; and #5559 from Cell Signaling Technology.

In one case, a binding agent comprises an antigen-binding site that selectively binds to TfR and an antigen-binding site that selectively binds to parkin. Examples of agents that comprise an antigen-binding site that selectively binds to parkin include, but are not limited to, alpha-synuclein, CASK, CUL1, FBXW7, GPR37, HSPA1A, HSPA8, multisynthetase complex auxiliary component p38, PDCD2, SEPT5, SNCAIP, STUB1, SYT11, and ubiquitin C, as well as monoclonal antibodies and commercially available antibodies, such as ab77924, ab233434, ab15494, ab7296, and ab73015 from Abcam; PA5-13399, 702785, 711820, PA5-13398, and 39-0900 from ThermoFisher Scientific; and #2132 from Cell Signaling Technology.

In one case, a binding agent comprises an antigen-binding site that selectively binds to TfR and an antigen-binding site that selectively binds to presenilin 1. Examples of agents that comprise an antigen-binding site that selectively binds to presenilin 1 include, but are not limited to, BCL2, CTNNB1, CTNND1, FLNB, GFAP, delta catenin, ICAM5, KCNIP3, NCSTN, PKP4, and UBQLN1, as well as monoclonal antibodies and commercial antibodies, such as AF149, MAB149, BAF166, AF166, BAF149, and MAB1491 from R&D Systems; ab76083, ab15456, ab227985, ab15458, ab227070, and ab15458 from Abcam; and GTX101028 from GeneTex.

In one case, a binding agent comprises an antigen-binding site that selectively binds to TfR and an antigen-binding site that selectively binds to presenilin 2. Examples of agents that comprise an antigen-binding site that selectively binds to presenilin 2 include, but are not limited to, BCL2-like 1, CAPN1, CIB1, calsenilin, FHL2, FLNB, KCNIP4, nicastrin, and UBQLN1, as well as monoclonal antibodies and commercial antibodies, such as AF153, BAF197, AF197, or MAB1218 from R&D systems; ab51249, ab15549, ab15548, and ab239841 from Abcam; or APS 21 from Novus Biologicals.

In one case, a binding agent comprises an antigen-binding site that selectively binds to TfR and an antigen-binding site that selectively binds to DR6. Examples of agents that comprise an antigen-binding site that selectively binds to DR6 include, but are not limited to, tumor necrosis factors as well as monoclonal antibodies and commercial antibodies, such as AF144 or BAF144 from R&D Systems; ab14740, ab8417, or ab214466 from Abcam; and LS-B10688, LS-C296886, and LS-C44700 from LSBio.

In one case, a binding agent comprises an antigen-binding site that selectively binds to TfR and an antigen-binding site that selectively binds to amyloid precursor protein (APP). Examples of agents that comprise an antigen-binding site that selectively binds to APP include, but are not limited to, reelin, APBA1, APBA2, APBA3, APBB1, APPBP1, APPBP2, BCAP31, BLMH, CLSTN1, CAV1, COL25A1, FBLN1, GSN, HSD17B10, and SHC1, as well as monoclonal antibodies and commercial antibodies, such as ab32136, ab 15272, ab12266, and ab220793 from Abcam; GTX101336 from GeneTex; and OMA1-03132, PA1-84165, 13-9749-82, or 41-9749-82 from ThermoFisher Scientific.

In one case, a binding agent comprises an antigen-binding site that selectively binds to TfR and an antigen-binding site that selectively binds to p75NTR. Examples of agents that comprise an antigen-binding site that selectively binds to p75NTR include, but are not limited to, FSCN1, MAGEH1, NDN, NGFRAP1, NGF, PRKACB, TRAF2, and TRAF4, as well as monoclonal antibodies and commercial antibodies, such as 55014-1-AP from Proteintech; #2693 from Cell Signaling Technology; and 07-476 from Sigma-Aldrich.

In one case, a binding agent comprises an antigen-binding site that selectively binds to TfR and an antigen-binding site that selectively binds to caspase 6. Examples of agents that comprise an antigen-binding site that selectively binds to caspase 6 include, but are not limited to, caspase 8; as well as monoclonal antibodies and commercial antibodies, for example, EP1325Y and EPR4405 from Abcam; NBP1-87683 from Novus Biologicals; 600-401-AD7 from Rockland Immunochemicals; and M02631 from BoosterBio.

In one case, a binding agent comprises an antigen-binding site that selectively binds to TfR and an antigen-binding site that selectively binds to myelin oligodendrocyte glycoprotein (MOG). Examples of agents that comprise an antigen-binding site that selectively binds to myelin oligodendrocyte glycoprotein (MOG) include, but are not limited to, monoclonal antibodies and commercial antibodies, such as MAB5680 from Sigma Aldrich.

In one case, a binding agent comprises an antigen-binding site that selectively binds to TfR and an antigen-binding site that selectively binds to proteolipid protein (PLP). Examples of agents that comprise an antigen-binding site that selectively binds to proteolipid protein (PLP) include, but are not limited to, monoclonal antibodies and commercial antibodies, such as NBP1-87781, NBP1-60071, NBP1-50309, and NB100-1608 from Novus Biologics; and ab28486 from Abcam.

In one case, a binding agent comprises an antigen-binding site that selectively binds to TfR and an antigen-binding site that selectively binds to Tropomyosin receptor kinase A (TRKA). Examples of agents that comprise an antigen-binding site that selectively binds to Tropomyosin receptor kinase A (TRK A) include, but are not limited to, monoclonal antibodies and commercial antibodies, such as EP1058Y, ab8871, ab86474, and ab109010 from Abcam; and AF175, AF1056, NBP2-67473, and NBP2-67841 from Novus Biologicals.

In one case, a binding agent comprises an antigen-binding site that selectively binds to TfR and an antigen-binding site that selectively binds to Tropomyosin receptor kinase B (TRKB). Examples of agents that comprise an antigen-binding site that selectively binds to Tropomyosin receptor kinase B (TRKB) include, but are not limited to, monoclonal antibodies and commercial antibodies, such as ab18987, ab33655, ab87041, and ab131483 from Abcam; and SC0556 and J. 977.7 from ThermoFisher and Invitrogen.

In one case, a binding agent comprises an antigen-binding site that selectively binds to TfR and an antigen-binding site that selectively binds to Tropomyosin receptor kinase C (TRKC). Examples of agents that comprise an antigen-binding site that selectively binds to Tropomyosin receptor kinase C (TRKC) include, but are not limited to, monoclonal antibodies and commercial antibodies, such as ab43078, ab227289, and ab227164 from Abcam; MAB-15543 and 701985 from ThermoFisher; and MAB3731 from R&D Systems.

In one case, a binding agent comprises an antigen-binding site that selectively binds to TfR and an antigen-binding site that selectively binds to myelin-associated glycoprotein (MAG). Examples of agents that comprise an antigen-binding site that selectively binds to myelin-associated glycoprotein (MAG) include, but are not limited to, monoclonal antibodies and commercial antibodies, such as MAB538 and MAB5381 from R&D Systems; ab89780, EP9714, ab203060, and ab187760 from Abcam; and SC-166849 from Santa Cruz Biotechnologies.

In one case, a binding agent comprises an antigen-binding site that selectively binds to TfR and an antigen-binding site that selectively binds to a synucleins. Examples of agents that comprise an antigen-binding site that selectively binds to a synucleins include, but are not limited to, monoclonal antibodies and commercial antibodies, such as ab51253, ab138501, ab27766, and ab209420 from Abcam; and NBP2-15365, NBP2-25146, NBP1-26380, and NBP1-05194 from Novus Biologicals.

In one case, a binding agent comprises an antigen-binding site that selectively binds to TfR and an antigen-binding site that selectively binds to p synucleins. Examples of agents that comprise an antigen-binding site that selectively binds to p synucleins include, but are not limited to, monoclonal antibodies and commercial antibodies, such as ab199304, ab199086, ab76111, ab6151, and ab221908 from Abcam.

In one case, a binding agent comprises an antigen-binding site that selectively binds to TfR and an antigen-binding site that selectively binds to gamma synucleins. Examples of agents that comprise an antigen-binding site that selectively binds to gamma synucleins include, but are not limited to, monoclonal antibodies and commercial antibodies, such as ab52633 and ab247328 from Abcam.

In one case, a binding agent comprises an antigen-binding site that selectively binds to TfR and an antigen-binding site that selectively binds to Tau. Examples of agents that comprise an antigen-binding site that selectively binds to Tau include, but are not limited to, monoclonal antibodies and commercial antibodies, such as ab109390, ab92676, ab32057, and ab151559 from Abcam; SC-32274 from Santa Cruz Biotechnologies; and MA5-15108, AHB0042, and MA5-12808 from Thermo Fisher Scientific.

In one case, a binding agent comprises an antigen-binding site that selectively binds to TfR and an antigen-binding site that selectively binds to vascular endothelial growth factor (VEGF). Examples of agents that comprise an antigen-binding site that selectively binds to vascular endothelial growth factor (VEGF) include, but are not limited to, monoclonal antibodies and commercial antibodies, such as Bevacizumab; Ranibizumab; ab8087, ab8086, and ab243850 from Abcam; and sc-7269 from Santa Cruz Biotechnologies.

In one case, a binding agent comprises an antigen-binding site that selectively binds to TfR and an antigen-binding site that selectively binds to neuropilin. Examples of agents that comprise an antigen-binding site that selectively binds to neuropilin include, but are not limited to, monoclonal antibodies and commercial antibodies, such as ab81321, ab184783, ab209445 and ab198323 from Abcam; and MA5-32179, MA5-32870, 12-3041-82, and 25-3041-82 from Thermo Fisher Scientific.

In one case, a binding agent comprises an antigen-binding site that selectively binds to TfR and an antigen-binding site that selectively binds to myelin basic protein (MBP). Examples of agents that comprise an antigen-binding site that selectively binds to myelin basic protein (MBP) include, but are not limited to, monoclonal antibodies and commercial antibodies, such as PA1050 from Boster and LS-B4814 from LSBio.

In one case, a binding agent comprises an antigen-binding site that selectively binds to TfR and an antigen-binding site that selectively binds to aquaporin 4. Examples of agents that comprise an antigen-binding site that selectively binds to aquaporin 4 include, but are not limited to, monoclonal antibodies and commercial antibodies, such as ab128906, ab128906, and ab248213 from Abcam.

In one case, a binding agent comprises an antigen-binding site that selectively binds to TfR and an antigen-binding site that selectively binds to glutamate receptor. Examples of agents that comprise an antigen-binding site that selectively binds to glutamate receptor include, but are not limited to, monoclonal antibodies and commercial antibodies, such as G9282, WH0002890M1, WH0002911M2, WH0002916M1, and WH0002918M6 from Millipore Sigma.

In one case, a binding agent comprises an antigen-binding site that selectively binds to TfR and an antigen-binding site that selectively binds to a Semaphorin (e.g., Semaphorin 3A, Semaphorin 4A, or Semaphorin 6A). Examples of agents that comprise an antigen-binding site that selectively binds to a Semaphorin (e.g., Semaphorin 3A, Semaphorin 4A, or Semaphorin 6A) include, but are not limited to, monoclonal antibodies and commercial antibodies, such as MAB1250 from R&D Systems; MBS607247 from MyBiosource; IC1250A and H00010501-M01 from Novus Biologics; and HIAT-2 from TransGenic, Inc.

### Pharmaceutical Compositions and Medicaments

Also described herein are pharmaceutical compositions and medicaments. Such pharmaceutical compositions and medicaments can comprise an active agent as described herein. Exemplary active agents include, an antibody or antigen-binding fragment thereof, a modified antibody or antigen-binding fragment thereof, or a bivalent or multivalent binding agent as described herein. In some cases, a pharmaceutical composition or medicament can comprise an antibody or antigen-binding fragment thereof as described herein. In some cases, a pharmaceutical composition or medicament can comprise a modified antibody or antigen-binding fragment thereof as described herein. In some cases, a pharmaceutical composition or medicament can comprise a binding agent as specified herein.

A therapeutic effect can be derived from the ability of the active agent to bind TfR and effectively cross a blood-tissue barrier such as the blood-brain barrier.

In some cases, an active agent as described herein can provide a therapeutic effect. Such a therapeutic effect can be mediated by a bivalent or multivalent nature of the active agent. For example, in some cases, once an active agent crosses a blood-tissue barrier via TfR binding, it can bind another molecule, wherein the other molecule can be other than TfR, such that binding the other molecule provides a therapeutic effect.

In some cases, an active agent as described herein can act as a carrier to transport a therapeutic agent across a blood-tissue barrier. Such a therapeutic agent can be a biologic or a small molecule. For example, a therapeutic agent can be another antibody or antigen binding fragment, a radioisotope, a chemotherapeutic, a steroid, a non-steroid anti-inflammatory drug, an antibiotic, an antifungal drug, an antiparasitic drug, an antiviral drug, a siRNA, a gene therapy vector, a viral vector, a nanoparticle, a hormone, a protein, a DNA, an RNA, or another therapeutic agent. Non-limiting examples of such therapeutic agents are described in more detail above.

A therapeutic agent can be encapsulated or contained in or on a nanoparticle. Such a nanoparticle can be conjugated to or otherwise attached to the active agent. In some cases, a nanoparticle can enhance solubility of the therapeutic agent, control the rate of release or metabolism of the therapeutic agent, stabilize the therapeutic agent, provide a means of attaching the therapeutic agent to the active agent, or provide other benefits.

A therapeutic agent which has been carried across a blood-tissue barrier by an active agent can provide a therapeutic effect. In some cases, the therapeutic agent can separate from, unbind from, or dissociate from the active agent in order to exert a therapeutic effect. In some cases, the therapeutic agent can provide a therapeutic effect while associated with, bound to, or conjugated to the active agent.

In cases where the therapeutic agent is bivalent or multivalent, it can bind to a molecule that is the target of the carried therapeutic agent or to a molecule that is near the target of the carried therapeutic agent. For example, if the active agent is carrying a therapeutic agent directed toward amyloid β plaques in a subject suspected of having Alzheimer's disease, then the antibody can bind an amyloid β plaque, thus positioning the therapeutic agent at its target.

Provided herein is a pharmaceutical compositions or a medicament that comprises a therapeutically effective amount of an antibody or antigen-binding fragment thereof, a modified antibody or antigen-binding fragment thereof, or a binding agent described herein, and a pharmaceutically acceptable carrier or excipient, for use in treating a neurological disease or disorder, or a central nervous system disease or disorder.

The phrase “pharmaceutically acceptable carrier or excipient” refers to molecular entities that do not materially affect the composition or change the active agent(s) contained therein, are physiologically tolerable, and do not typically produce an allergic reaction, or similar untoward reaction, when administered to a subject.

The pharmaceutical compositions or medicaments to be used for in vivo administration as described herein may, in some instances, be sterilized. This may be accomplished by, for example, filtration through sterile filtration membranes, or any other suitable method for sterilization. Other methods for sterilization and filtration are within the scope of the present disclosure.

### Uses and Methods

In some cases, an antibody or antigen-binding fragment thereof, described herein can be utilized to determine that a TfR is present in a biological sample obtained from a subject. A biological sample can be preserved or treated prior to conducting the method as needed. In one non-limiting example, a blood sample obtained from a subject can be treated with heparin to prevent clotting. In another non-limiting example, a tissue sample can be frozen and sectioned prior to conducting immunohistochemistry. Determination that a TfR is present in the biological sample can be conducted using any suitable method including, but not limited to, immunohistochemistry, FACS, ELISA, ELISPOT, western blot, etc.

In some cases, an active agent can be used in the treatment of a neurological disease, a central nervous system disease, or a combination thereof. As used herein, an “active agent” may be any of the antibodies, antigen-binding fragments, modified antibodies, modified antigen-binding fragments, or binding agents described herein.

The terms “treat,” “treating,” and “treatment” as used herein encompass improvement in any one of the symptoms or outcomes that may be measured according to any suitable test or any of the tests described herein.

A “therapeutically effective amount” or “therapeutically effective dose” are used interchangeably herein and refer to an amount of an active agent that provokes a therapeutic or desired response in a subject. In some cases, the therapeutic or desired response is the alleviation of one or more symptoms associated with a disease or disorder. In some cases, a therapeutic or desired response comprises prophylactic treatment of a disease or a disorder. An active agent may be administered in a dose that is sufficient to cause a therapeutic benefit to the subject. The dose may vary depending on a variety of factors including the active agent selected for use, and the age, weight, height and/or general health of a subject to be treated.

An active agent described herein can be administered to a subject in need thereof. Such a subject can be a mammal, for example, a pig, dog, cat, rat, mouse, guinea pig, hamster, woodchuck, squirrel, rabbit, or human. A subject may have a disease or disorder, such as a disease or disorder, for example, of the brain, nervous system, retina, placenta, testis, thymus, or involving the placenta. An animal subject that is not a human subject can be an animal model of a human disease or disorder, for example, of the brain, nervous system, retina, placenta, testis, thymus, or involving the placenta.

In some cases, an active agent can be used for transporting an agent across the blood-brain-barrier or into the CNS of a subject.

In some cases, an active agent can be used in the manufacture of a medicament for transporting an agent across the blood-brain-barrier or into the CNS of a subject.

In some cases, an active agent described herein can be used for the treatment of a neurological disease, a central nervous system (CNS) disease, or a combination thereof.

A neurological disease, or a central nervous system disease, can comprise, in some instances, Alzheimer's disease (AD), a stroke, dementia, muscular dystrophy (MD), multiple sclerosis (MS), amyotrophic lateral sclerosis (ALS), Angelman syndrome, Liddle syndrome, Parkinson's disease, Pick's disease, Paget's disease, a cancer or a metastasis thereof, Bell's palsy, or a combination thereof.

Diseases and disorders that affect the central nervous system (CNS), and which can be treated with the methods described herein include, but are not limited to, Bell's palsy, cerebral palsy, epilepsy, Alzheimer's disease, motor neurone disease (MND), multiple sclerosis (MS), a neurofibromatosis, Parkinson's disease, stroke, sciatica, and shingles.

Diseases which can be treated with the methods described herein include, but are not limited to, a metabolic disease. A metabolic disease may include, for example, non-alcoholic steatohepatitis (NASH), non-alcoholic fatty liver disease (NAFLD), diabetes mellitus type 1 (also known as type 1 diabetes), diabetes mellitus type 2, insulin resistance (IR), obesity, Prader-Willi syndrome (PWS), cardiovascular disease, or a combination thereof.

Diseases which can be treated with the methods described herein include, but are not limited to, a neuroendocrine disease. A neuroendocrine disease may include, for example, erectile dysfunction (ED), also from aging, of pituitary/hypothalamic origin; premature ejaculation (PE), of pituitary/hypothalamic origin, and for treating or ameliorating dysfunctions associated with such disorders; a neuroendocrine tumor (NET).

A cancer to be treated using the methods described herein includes, but is not limited to, a solid tumor or a semi-solid tumor; a tumor can be a primary tumor or a metastatic tumor. Exemplary cancers to be treated using a method described herein include, but are not limited to, a kidney cancer (e.g., a renal cell carcinoma), a lung cancer (e.g., a primary non-small cell lung cancer, or a metastatic non-small cell lung cancer), melanoma (e.g., a primary melanoma, an unresectable melanoma or a metastatic melanoma), a head and neck cancer (e.g., a head and neck squamous cell carcinoma), a Merkel cell carcinoma, a urothelial cancer (e.g., a locally advanced urothelial cancer or a metastatic urothelial cancer), a breast cancer, a pancreatic cancer, an ovarian cancer, a uterine cancer, a colorectal cancer, a prostate cancer, a bladder cancer, a liver cancer, a sarcoma, a myeloma, and a lymphoma, or a metastasis of any of such tumors. In one instance, the cancer is a sarcoma or a metastasis thereof including, but not limited to, a uterine sarcoma, an angiosarcoma, a stromal sarcoma, an osteosarcoma, a chondrosarcoma, a leiomyosarcoma, a metastasis of any thereof, or a combination thereof. In another instance, the cancer is a carcinoma or a metastasis thereof including, but not limited to, a carcinoma of a cervix, a lung, a prostate, a breast, head and neck, a colon, a liver or an ovary, an adenocarcinoma, an adenosquamous carcinoma, a papillary serous adenocarcinoma, and a clear cell adenocarcinoma.

In another instance, the cancer is a brain cancer or a metastasis thereof including, but not limited to, a glioblastoma multiforme (GBM), a glioma, an astrocytoma, a meningioma, a pituitary tumor, a craniopharyngioma, a germ cell tumor, a pineal region tumor, a medulloblastoma, and a primary CNS lymphoma. In another instance, the cancer is a breast cancer or a metastasis thereof including, but not limited to, a luminal A cancer, luminal B, triple negative/basal-like, or HER2 type. Non-limiting examples of luminal A breast cancers include, for example, ER+ and/or PR+, HER2−, and low Ki67 breast cancers. Non-limiting examples of luminal B breast cancers include, for example, ER+ and/or PR+, HER2+, or HER2− with high Ki67 breast cancers. Non-limiting examples of triple negative/basal-like breast cancers include, for example, ER−, PR−, HER2− breast cancers. Non-limiting examples of HER2 type breast cancers include, for example, ER−, PR−, HER2+ breast cancers. In another instance, the cancer is a colorectal cancer or a metastasis thereof including, but not limited to, a cancer of a colon, a rectum (anus), or an appendix. In another instance, the cancer is a lung cancer or a metastasis thereof including, but not limited to, a non-small cell lung cancer or a small cell lung cancer. In another instance, the cancer is a kidney cancer or a metastasis thereof including, but not limited to, a renal cell carcinoma (e.g., a clear cell renal cell carcinoma, a papillary renal cell carcinoma, or a chromophobe renal cell carcinoma), a transitional cell carcinoma, a Wilms tumor (nephroblastoma), or a renal cell sarcoma.

Administration

A subject can be administered an active agent, if the subject is suspected of needing a therapeutic effect conferred by the active agent. In some instances, a therapeutic effect may be observed when a therapeutic agent is conjugated thereto. In other cases, an increased therapeutic effect is observed when an active agent is conjugated to a therapeutic agent. For example, an increased therapeutic effect may be a synergistic effect. In some instances, administration of a multimeric active agent described herein results in reduced side effects and/or immunotoxicity in a subject compared to administration of separate agents.

In some cases, the active agent can be administered through an injection (e.g., intravenously, intraperitoneally, subcutaneously, intramuscularly, etc.). Accordingly, these antibodies or antibody fragments can be combined with pharmaceutically acceptable vehicles such as saline, Ringer's solution, dextrose solution, and the like. The particular dosage regimen, i.e., dose, timing and repetition, can depend on the particular individual, the disease or disorder, or the subject's medical history.

In some cases, the dose administered to a subject can be dependent on the subject's transferrin levels. Some health or disease states can be capable of modulating the levels of free transferrin in the blood. Transferrin can compete with an antibody or antibody fragment described herein for binding, so the dose may depend on the transferrin level. For example, the dose can be a function of the transferrin level. In some cases, the dose can be proportional to the transferrin level. In some cases, transferrin level can be monitored over time, and the dose can be adjusted accordingly. In some cases, the dose can be increased if the transferrin level increases. In some cases, the dose can be decreased if the transferrin level decreases.

A subject can be administered one or more doses of an antibody or antibody fragment thereof, a modified antibody or antigen-binding fragment thereof, or a binding agent described herein until the subject exhibits improvement in one or more symptoms of the disease or disorder. Treatment includes partial or complete treatment of one or more symptoms of the disease or disorder.

Some health or disease states can be capable of modulating the levels of TfR expressed in one or more tissues. For example, a subject can have a dysregulation (upregulation or downregulation) of transferrin, which can be caused, for example, by alteration of a promoter or promoter activity, changes in signaling, or a mutation in a promoter. Such a change in TfR expression can result in a different amount of antibody or antigen binding fragment binding to the TfR.

Some health or disease states can be capable of changing the binding affinity between transferrin and the TfR. For example, a mutation or mis-folding of either transferrin or the TfR can cause a change in affinity between these two molecules. Such a change can alter the amount of antibody or antigen binding fragment which can bind to the TfR. This can occur due to a change in the competition for binding.

Some health or disease states can be capable of changing the binding affinity between the TfR and an antibody or antigen binding fragment as described herein. For example, some health or disease states can affect the tertiary or quaternary structure of the TfR, which can alter how TfR interacts with other molecules, including antibodies or antigen binding fragments.

In some cases, a subject can have a mutation in a gene coding for TfR. Such a mutation can increase or decrease the affinity of the TfR for transferrin. In certain embodiments, such a mutation can increase or decrease the affinity of the TfR for an antibody or antigen-binding fragment. If a gene coding for TfR comprises a mutation, the dose of an antibody or antigen-binding fragment can be adjusted.

In some cases, a subject can have a mutation in the transferrin gene. Such a mutation can increase or decrease the affinity of the TfR for transferrin. In some cases, a difference in affinity between the TfR and transferrin can affect the amount of antibody or antigen-binding fragment endocytosed, as transferrin can compete for the antibody or antigen-binding fragment. In such cases, the dose of the antibody or antigen-binding fragment can be adjusted accordingly.

In some cases, a subject may have a neurological disease or disorder, a CNS disease or disorder, and/or a cancer or a metastasis thereof, but may have a normal level of transferrin and/or a TfR. In such cases, a multivalent binding agent as described herein may utilize a first antigen-binding fragment that selectively binds to TfR to transport the binding agent across a barrier to a site in the body such that the binding agent is released and a second antigen binding fragment that binds to a particular target antigen of interest for the disease or disorder. The second antigen binding fragment may itself exert a therapeutic effect, or the binding agent may be labeled with a therapeutic label that exerts a therapeutic effect.

In some cases, a subject can be administered one or more doses of an antibody or antigen-binding fragment, or a binding agent. In some cases, a dose can be a general dose which can be administered to any of a type of subject (e.g., human, pig, cow, etc.). In some cases, a dose can depend on the body weight of a subject. For example, a subject with a higher body weight might receive a higher dose of an antibody or antigen-binding fragment, or a binding agent. In some cases, the dose can depend on one or more biochemical or physiological aspects of the subject. For example, a dose can depend on a subject's fitness, immune function, nutritional status, general health, ability to metabolize the antibody or antigen-binding fragment, binding agent or an adjuvant, or other aspect.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

Reference to the term “antibody” in the examples is inclusive of an “antigen-binding fragment thereof.”

### Example 1: TFR Light Chain Mediated Binding Molecules that Selectively Bind to TfR

Antibodies having at least a variable light region comprising a CDR1 (L1), a CDR2 (L2) and a CDR3 (L3), as well as a variable heavy region comprising a CDR1 (H1), a CDR2 (H2), and a CDR3 (H3) were produced using a step by step process to identify and characterize antibodies from our antibody library (see, FIG. 1). Reagents are quality control checked on an Octet QK to confirm biomolecular binding interactions and by SDS-PAGE to verify the protein size and purity. Then the panning process is initiated with a variable light chain (VK) only single-chain variable fragment (scFv) antibody phage library using a KingFisher Instrument as described:

Step 1. Panning begins with deselections, depleting the library for magnetic bead binders, protein tag binders, binders to proteins with high homology to the target antigen, and hydrophobic binders. In this step, we want to remove any antibodies that bind to reagents and materials used in the panning process, not including the target antigen. For deselection, we maintain the same concentration of antigens throughout every round of panning.

Step 2. After 6 deselections, we take the remaining phage, add the target antigen and run the selection program, which has a variety of stringent selection pressures for affinity and thermostability. For selection, we decrease the concentration of the target antigen and shorten the selection times after each round of panning.

Step 3. Once selection is completed, we wash the phage with two different wash buffers (PBS and PBST). As the panning process is progressing, both the number of washes and duration of washes increases. In this step, we want to eliminate any low affinity binders with fast off-rates.

Step 4. When the washes are done, we elute the phage using triethylamine (TEA) in water. Then we add Tris-HCl pH 7.4 to neutralize the phage and infect chemically competent E. coli ER2738 cells using the entire the solution per panning arm condition.

Step 5. After infection, the E. coli cells with our antigen targeted scFv phage are concentrated, placed on an agar plate, and incubated overnight. In this step, we want to eliminate any cells that have not been infected with our phage plasmid vector. Once at least 3 rounds (usually 4 rounds) have 6been completed, the screening procedures were started.

Step 6. The next step in the work flow is to pick several plates worth of single colonies from the last panning round, Sanger Sequence the clones, and identify the unique antibodies to then reduce the number of clones needed to be screened. The unique scFv clones are screened using enzyme linked immunosorbent assays (ELISA), fluorescence-activated cell sorter (FACS), and/or The Carterra LSA surface plasmon resonance (SPR) Instrument for high throughput monoclonal antibody (mAb) characterization. Once the scFv screening process is complete, we then select the antigen specific binding molecules to reduce the antibody pool furthermore. The selected scFv's are then cloned into a modified pTT5 vector for full immunoglobulin G1 (IgG1) mammalian reformatting and expression. Finally, the purified IgGs are screened once again for cell binding on FACS as well as a functionality.

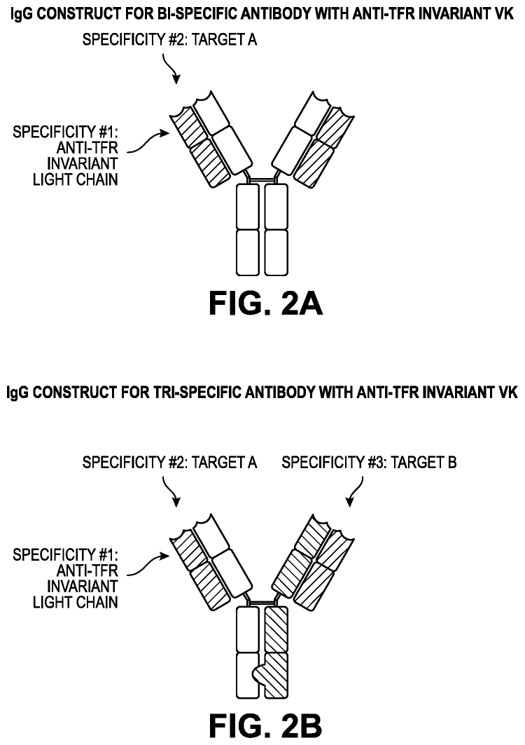

FIGS. 2A-B provide construct design for anti-TFR light chain bi-specific constructs. FIG. 2A illustrates the IgG1 antibody construct design for a bi-specific antibody with an anti-TFR invariant light chain (orange) and proprietary SuperHuman 2.0 (SH2.0) heavy chain variants (blue) for secondary specific binding. FIG. 2B illustrates the IgG1 antibody construct design for a tri-specific antibody with an anti-TFR invariant light chain (orange) and proprietary SH2.0 heavy chain variants (blue and green) for secondary and tertiary specific binding held together by a ‘Knobs-into-holes’ Fc constant heavy chain 3 (CH3) region.

### Example 2: Screening Methods

In order to confirm that the antibodies or antigen-binding fragments thereof, recognize human TfR, screening assays are carried out.

Immunoprecipitation

Five (5) mg of the antibody is immobilized with respect to 1 ml of CNBr-activated sepharose 4B in Glass Filter, to produce antibody beads. Subsequently, SKOV-3 cells cultured in a 10-cm³ dish are recovered to prepare 600 μL of a cell lysate. 60 μL of biotin id added to 600 μL of the cell lysate to biotinylate the antigen. 150 μL of a solution of the produced antibody beads and the biotinylated cell lysate are placed in a 2-mL tube, and the obtained mixture is then stirred at 4° C. for 6 hours. Thereafter, the tube is subjected to centrifugation (5500 g, 1 minute, 4° C.), and the supernatant is removed. Then, 800 μL of a washing buffer (0.5 mM Biotin and 0.1% Tween20/PBS) is added into the tube, and the beads are then washed by centrifugation. The beads are repeatedly washed three times, and 30 μL of a citric acid solution for elution (50 mM citric acid, pH 2.5) is then added thereto, followed by stirring and then centrifugation (5,500 g, 1 minute, 4° C.). An immune complex is eluted by recovering the supernatant. The elution operation is repeatedly performed three times, and the supernatant is recovered. It is neutralized by addition of 3 M Tris, and is then electrophoresed by SPS-PAGE. A band is confirmed by silver staining. This sample is also subjected to Western blotting using streptavidin-TRP (Anti-Streptavidin, IgG Fraction, Conjugated to Peroxidase, CORTEX Biochem). It is confirmed that antibodies tested with these methods bind to a protein with a molecular weight of approximately 90 KD (the molecular weight of TfR).

Enzyme Linked Immunosorbent Assay (ELISA)

The unique scFv clones identified from the panning process were screened by enzyme linked immunosorbent assay (ELISA) using the scFv periplasmic extract (PPE) as described below:

Step 1. The SH2.0 scFv's contain a V5 tag within its vector, which we use to capture the soluble scFv. Therefore, high protein binding plate(s) are coated overnight with an anti-V5 tag capture antibody. Once the incubation process is complete, the plate(s) are washed with buffer to remove any excess capture antibody not bound to the plate.

Step 2. Secondly, the plate(s) are then blocked with BSA to prevent any non-specific binding of the PPE. The plate(s) are again washed with buffer to remove extra blocking buffer.

Step 3. Next, the PPE is added to the plate(s) and incubated to be captured by the bound anti-V5 antibodies. Then the plate(s) are washed a few times with buffer to eliminate any non-binding soluble scFv.

Step 4. Then the plate(s) are coated with a predetermined concentration of tagged antigen to bind to the antibodies and detect the captured protein. The plate(s) are washed a few times with buffer to disregard any excess antigen.

Step 5. Afterward, a tag specific enzyme conjugated antibody is added to the plate(s) and will bind to the tagged antigens in each well. The plate(s) are wash one last time to remove any enzyme conjugated antibody not bound to the antigen.

Step 6. Finally, a colorimetric substrate is added to the wells such as Tetramethylbenzidine (TMB) and form a colored solution when catalyzed by the enzyme to illuminate the antigen specific binders. Before quantifying the absorbance with an ELISA reader, we add hydrochloric acid (HCl) to halt the colorimetric reaction. Once the absorbance is measured, the amount of protein in the samples is determined and the binders can be identified.

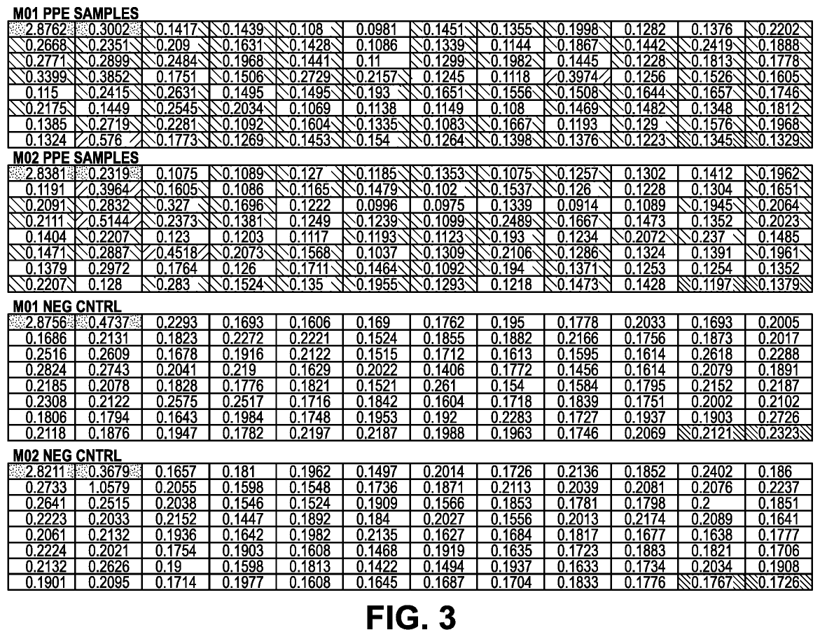

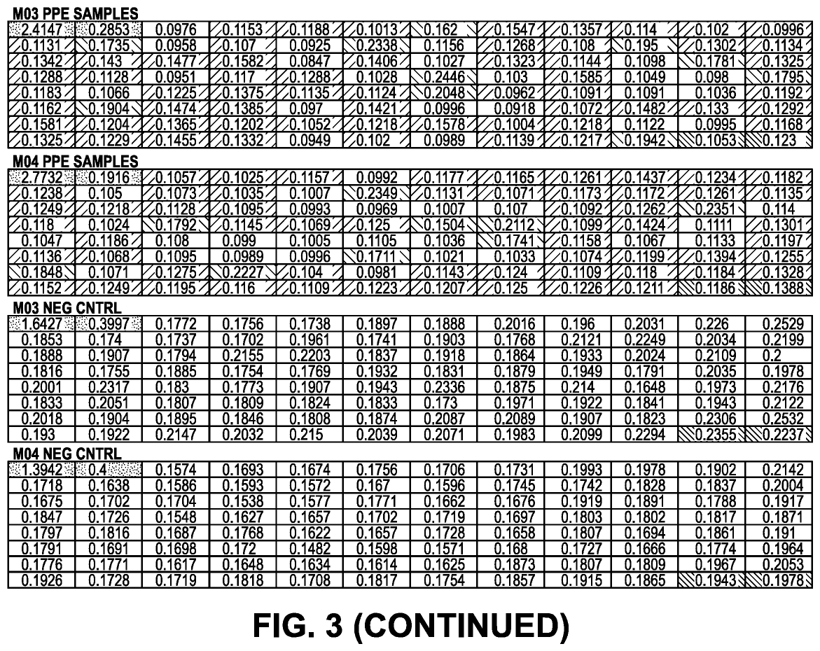

FIG. 3 provides data from the scFv periplasmic extract (PPE) screening methods by (Enzyme linked immunosorbent assay (ELISA).

Screening Methods FACS

The unique scFv antibodies identified after the panning process (as described in FIG. 1) are screened by fluorescence activated cell sorter (FACS) to confirm cell-based binding. The unique clones are tested against a cell line that overexpresses the antigen/receptor and a cell line that is negative for the antigen/receptor or expresses an off-target antigen/receptor using the PPE as described below:

Step 1. First, collect at least 10 million cells for each cell line per plate in order to have 100,000 cells per well of a 96 well plate and spin them down in a centrifuge to remove the media.

Step 1. Resuspend the cells in FACS buffer and aliquot them into the plate accordingly. Then spin the plate(s) to remove any remaining cell culture media.

Step 3. Next, add equal parts FACS buffer and scFv PPE and mix gently with the cells. Incubate the mixture on ice covered with foil for 1 hour.

Step 4. After the incubation is complete, wash the cells with FACS buffer to get rid of any PPE that is not binding to the cells. Then add an anti-myc conjugated antibody diluted in FACS buffer and incubate it on ice covered with foil for 30 minutes.

Step 5. Then wash the cells twice with FACS buffer. In this step, we want to eliminate as much excess conjugated antibody as possible to reduce the amount of background binding when absorbance is being measured.

Step 6. Lastly, resuspend the cells in FACS buffer and paraformaldehyde (PFA) in order to prevent the bound antibodies from internalizing into the cell and collect 10,000 events of the FACS analyzer.

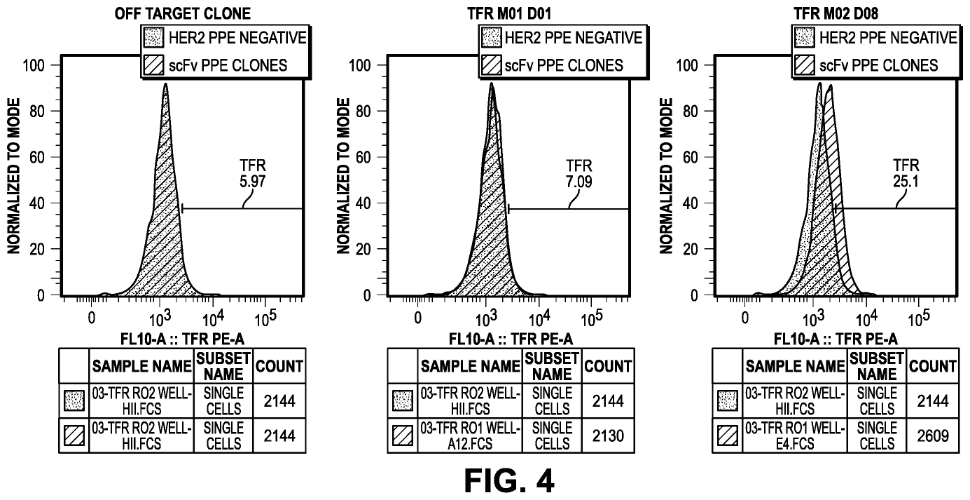

The results are shown in FIG. 4.

Construct Design for IgG1 Conversion

The construct design for both the heavy and light variable chains used to convert the confirmed scFv binders into full IgG1 antibodies.

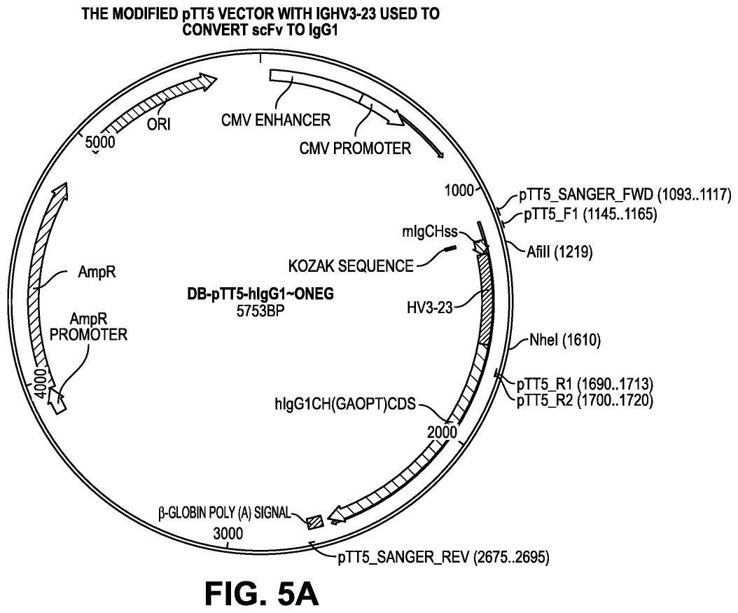

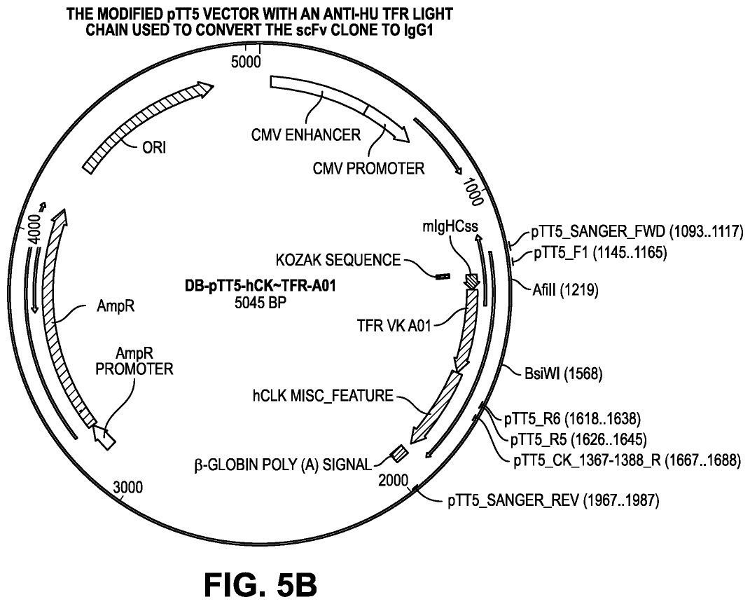

The modified IgG1 heavy chain pTT5 vector with an IgG1 constant heavy chain (purple) and an O negative IGHV3-23 variable domain (grey) inserted using AflII and NheI digestion enzymes is shown in FIG. 5A. The IGHV3-23 domain DNA sequence is shown below:

(SEQ ID NO: 116)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTCTCAGCT
ATTAGTGGTAGTGGTGGTAGCACATACTACGCAGACTCCGTGAAGGGCCG
GTTCACCATCTCCAGAGACAATTCCAAGAACACGCTGTATCTGCAAATGA
ACAGCCTGAGAGCCGAGGACACGGCCGTATATTACTGTGCGAAAGGCGGG
CGCGATGGGTATAAGGGCTACTTTGACTACTGGGGCCAAGGGACCCTGGT
CACCGTCTCCTCAG.

The modified IgG1 light chain pTT5 vector with a constant light chain region (blue) and an anti-TFR variable light chain (marron), which is a confirmed scFv binder, inserted using AflII and BsiWI digestion enzymes, is shown in FIG. 5B. The anti-TFR light chain DNA sequence is shown below:

(SEQ ID NO: 117)
GACATCCAGATGACCCAGTCTCCATCCTCCCTGTCTGCATCTGTAGGAGA
CAGAGTCACCATCACTTGCCGGGCAAGTCAGAACATTAATAAGAACTTAA
ATTGGTATCAGCAGAAACCAGGGAAAGCCCCTAAGCTCCTGATCTATAAG
GCATCCAGTTTGGAGAGTGGGGTCCCATCAAGGTTCAGTGGCAGTGGATC
TGGGACAGATTTCACTCTCACCATCAGCAGTCTGCAACCTGAAGATTTTG
CAACTTACTACTGTCAACAGGCAAAAAGTCTGCCTCTCACTTTCGGCGGA
GGGACCAAGGTGGAGATCAAA.

FACS Screening Methods

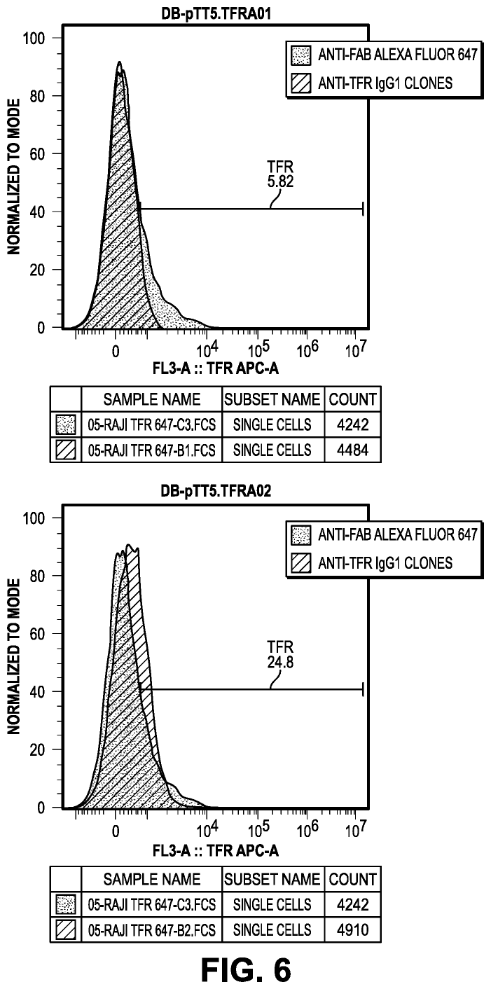

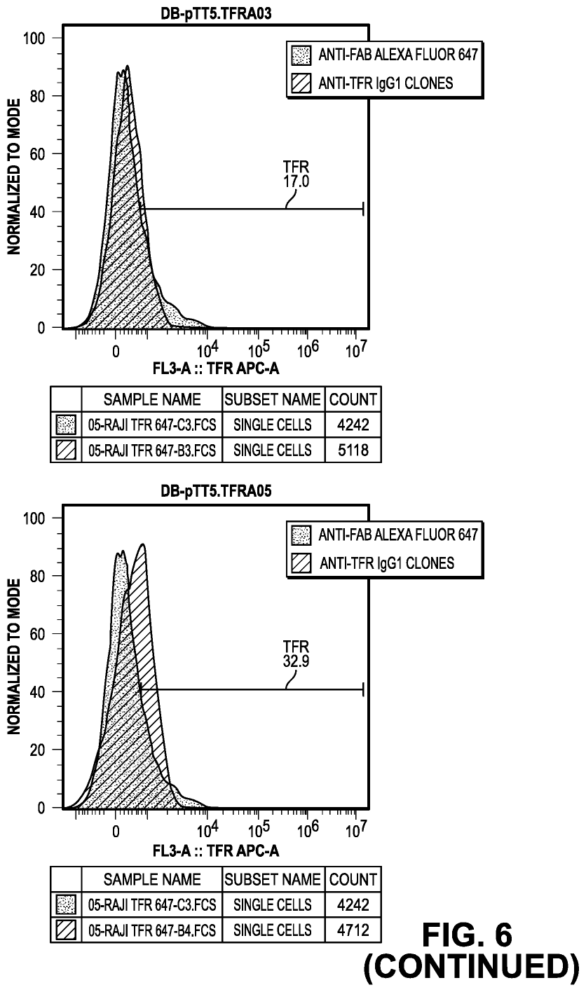

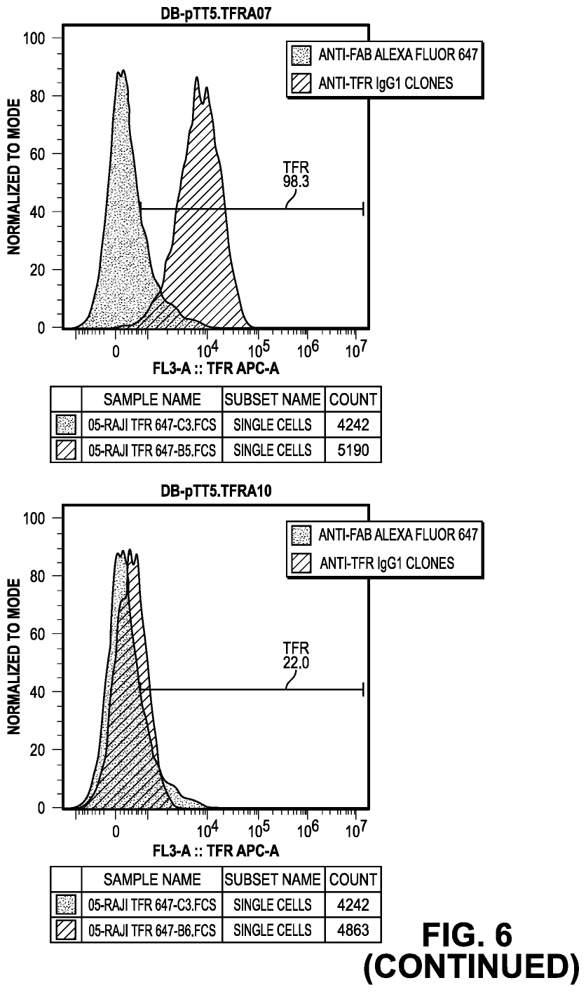

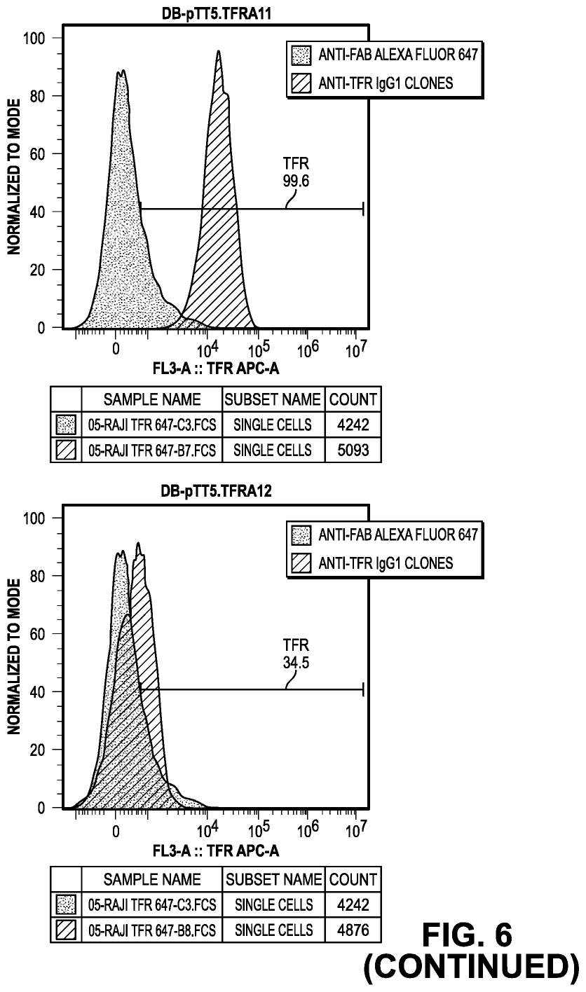

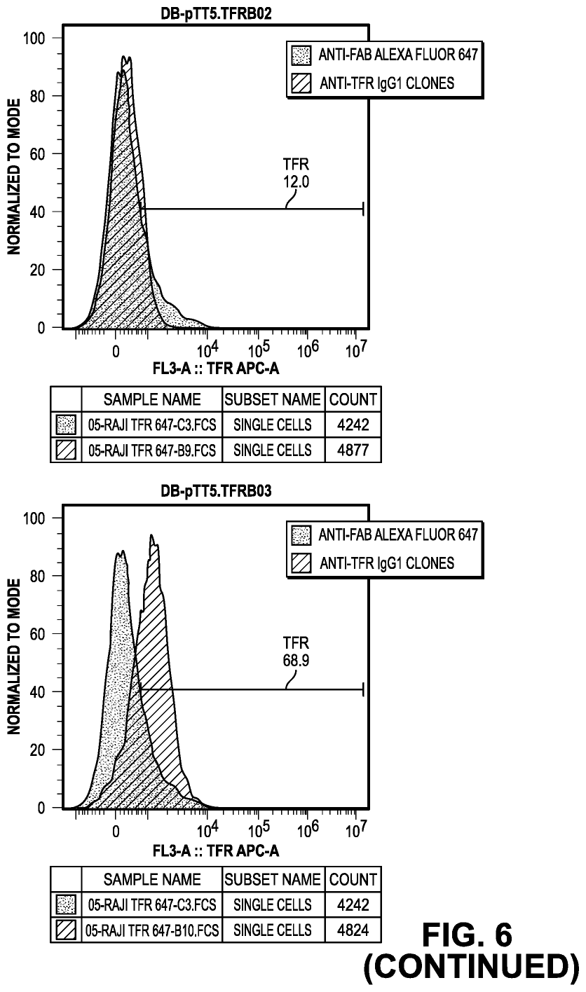

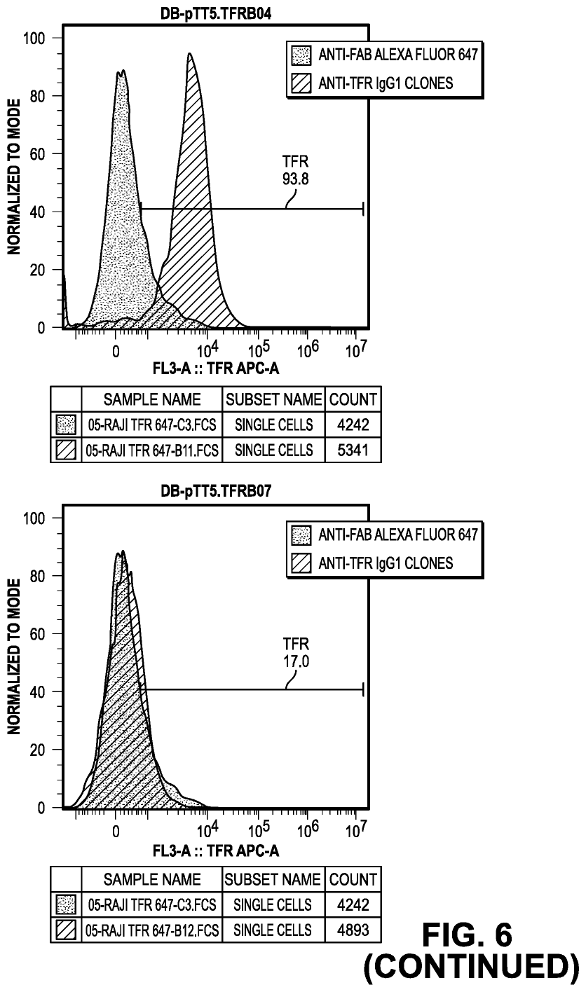

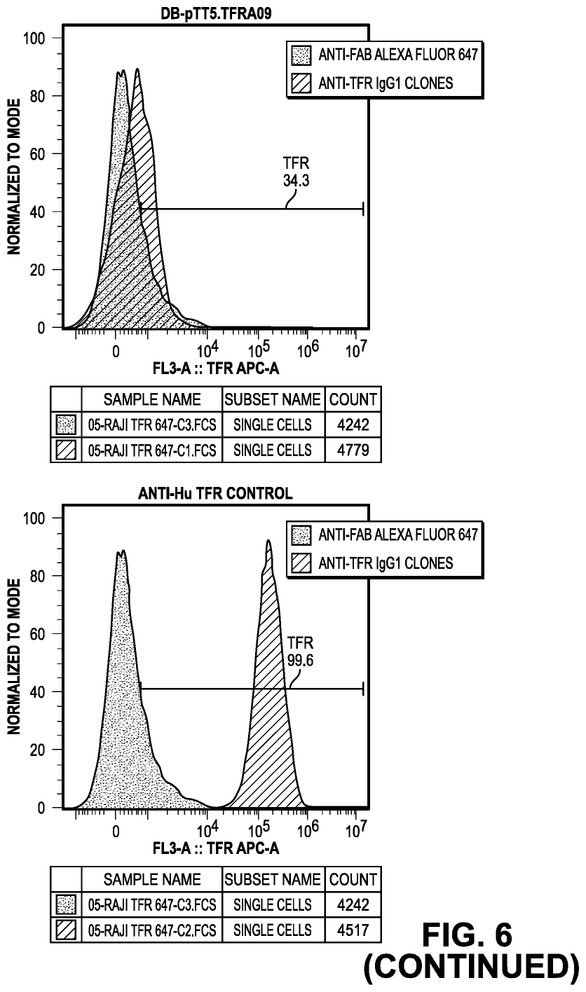

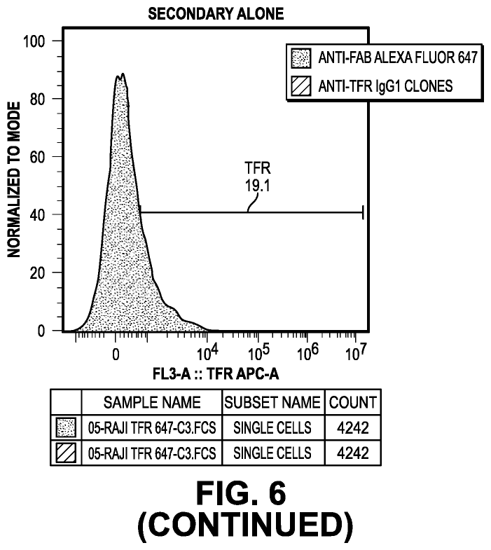

The TFR antigen specific binding scFv clones were reformatted into IgG1s and screened by FACS as described in FIG. 4 on Raji cells to confirm that the antibodies retained their binding profiles. An anti-Human Fab Alexa Fluor 647 was used as a negative control to measure background/non-specific binding. The results of the assay are provided in FIG. 6.

Cell Line Quality Control (QC) by FACS

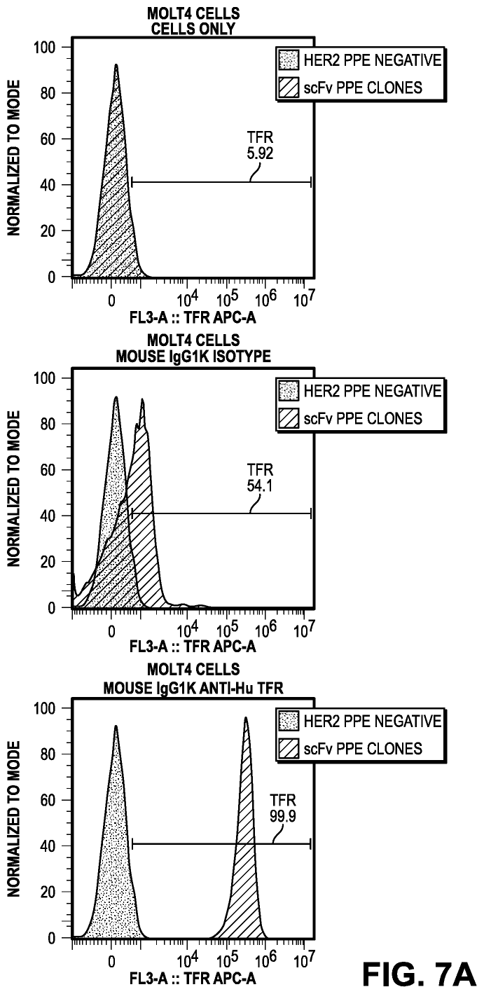

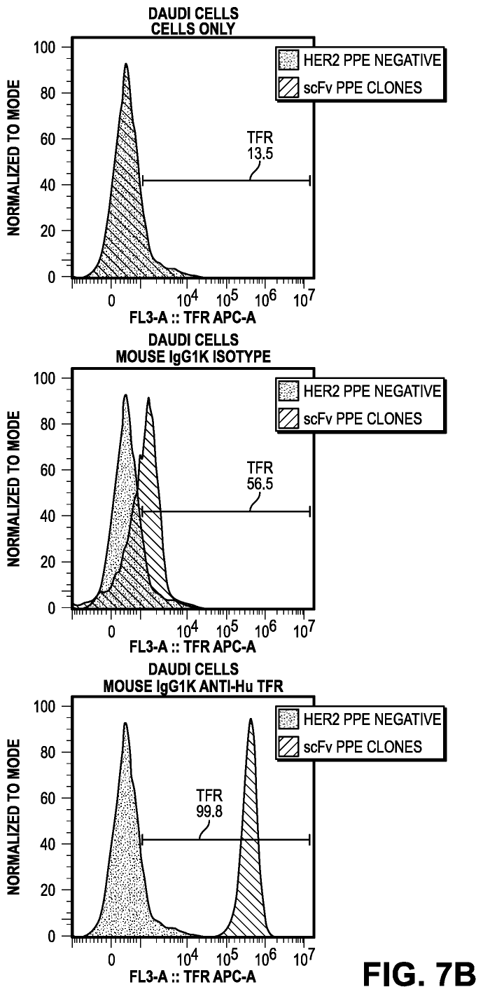

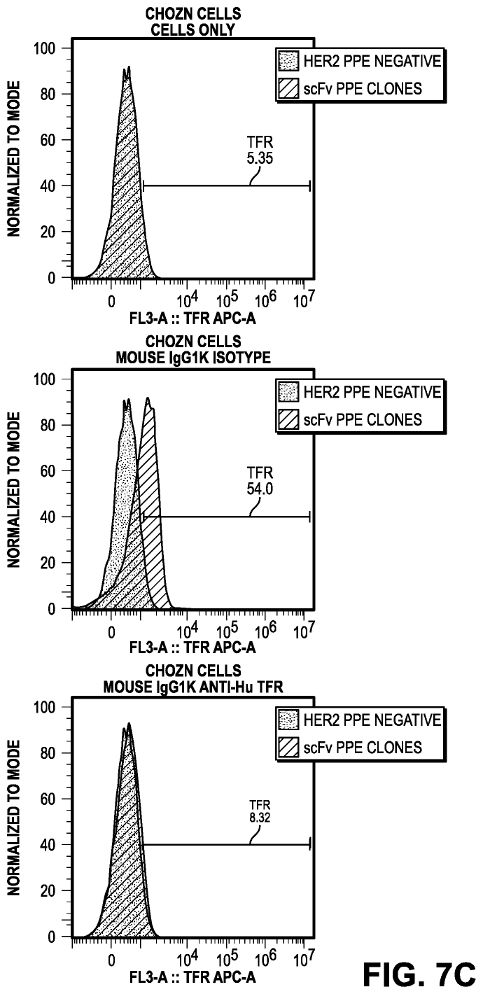

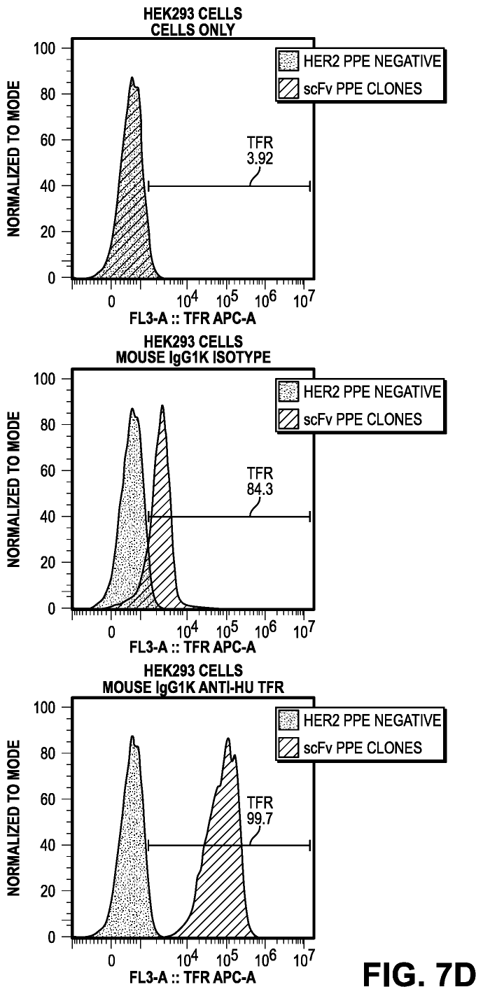

The cell lines used for functional assay screening methods were quality control (QC) checked by FACS to determine if the cells express TFR on their surface. The purpose of this QC is to find one cell line for a positive internalization assay and one cell line for a negative internalization assay. There were four cell lines tested, including Molt4 (A), Daudi (B), CHOZN (C), and HEK293 (D). Each cell line was tested against CD71 (Transferrin Receptor) Monoclonal Antibody (Invitrogen cat no. 16-0719-85) to test for receptor specific binding and Mouse IgG1 kappa Isotype Control (Invitrogen cat no. 16-4714-82) as a negative control to test for background/non-specific binding. (FIG. 7)

FACS Screening Methods

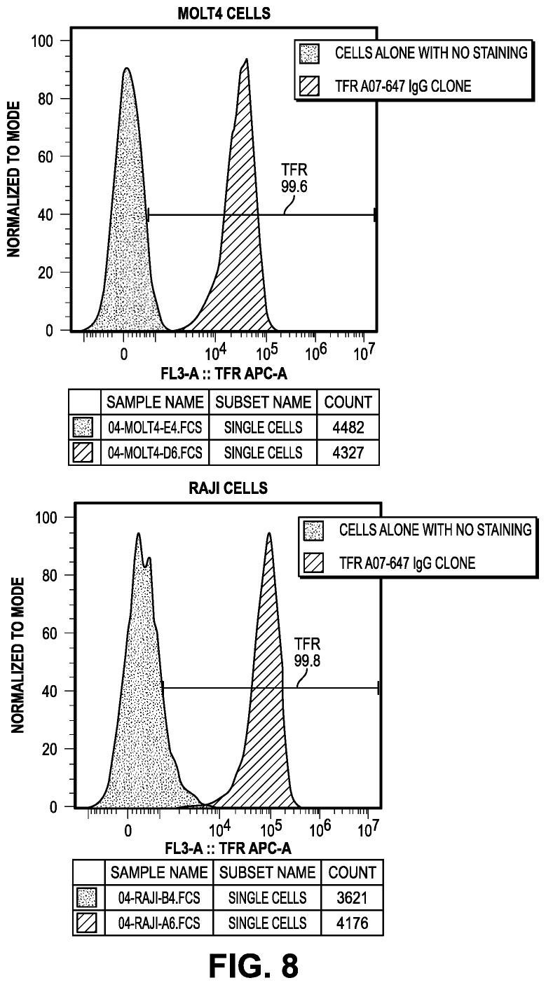

The anti-Human TFR IgG1 antibodies were screened by FACS on Raji and Molt4 cells as described in FIG. 4 to confirm binding after being conjugated with an Alexa Fluor. These antibodies were primary labelled with Alexa Fluor 647 using an Invitrogen antibody labeling kit (cat no. A20186) according to the manufacture's manual. (FIG. 8)

Internalization Screening Methods

The positive internalization assay was performed on the primary labeled anti-Human TFR IgG1 antibodies to test for receptor-mediated endocytic process in which the cell will only take in an extracellular molecule if it binds to its specific receptor on the cell's surface. This assay was done on Raji cells as described below:

Step 1. Count the appropriate cells, collect enough to have 1×106 cells per condition for each antibody, and wash the cells using FACS buffer (1×PBS+2% FBS).

Step 2. Then block the cells with 0.1% BSA in 1×PBS for 30 minutes at the appropriate temperature to prevent non-specific binding. All samples that are going to be incubated with the antibody at 37° C. should be blocked at 37° C. and all samples that are going to be incubated with the antibody at 4° C. should be blocked at 4° C. All samples must be protected from light during incubation periods from this point forward. All centrifuge steps are done at 4° C. and 1200 rpm.

Step 3. Add primary labeled antibodies at the appropriate concentration to saturate receptors on cells and incubate at 4° C. or 37° C. according to the chart and instructions below. Move samples to PCR strip tubes as needed.

| | |
|---|---|
| 4° C./30 min | Cells background no staining |
| 4° C./30 min Ab1 | Maximum external binding control |
| 4° C./30 min Ab1 + strip | Should be negative |
| (strip control) | |
| 37° C./5 min Ab1 + strip | |
| 37° C./15 min Ab1 + strip | |
| 37° C./30 min Ab1 + strip | |
| 37° C./60 min Ab1 + strip | |
| 37° C./120 min Ab1 + strip | Maximum internalization mAb within |
| | 2 hours |
| 37° C./120 min Ab1 | Maximum external + internalized Ab |

Start by adding antibody to the 120 min. sample tube; 1 hour later, add antibody to the 60 min. sample tube; 12 hour later, add antibody to all the 30 min. sample tubes, keeping the control tubes on ice; 15 min. later, add antibody to the 15 min. sample tube; 10 min. later, add antibody to the 5 min. sample tube; STOP all tubes after 5 min. is up; To stop internalization, collect all tubes and place directly into ice; and Move all samples to a 96 well u bottom plate.

Step 4. Wash the cells twice with ice cold FACS buffer.

Step 5. Strip the cells of any external binding by incubating the cells with Roswell Park Memorial Institute (RPMI) culture media at pH 2.8 for no longer than 10 min at room temperature.

Step 6. Wash the cells twice with ice cold FACS buffer, resuspend them in FACS buffer, and collect at least 10,000 events on the FACS analyzer.

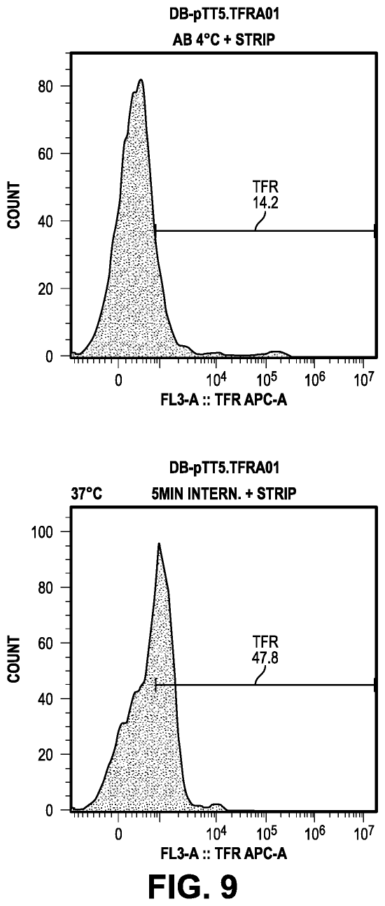

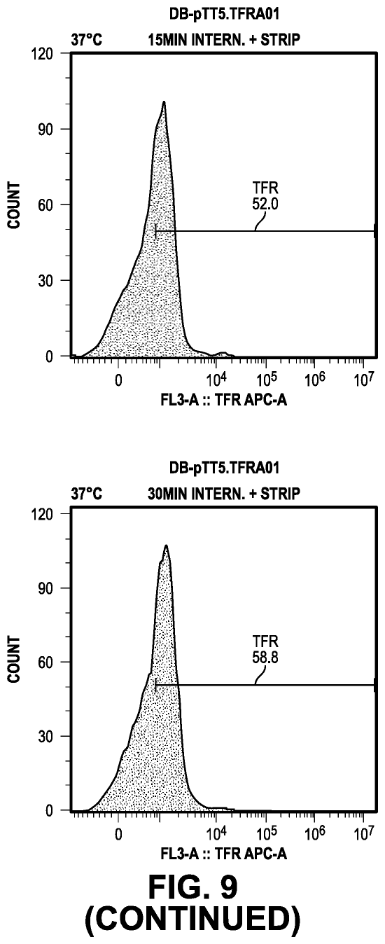

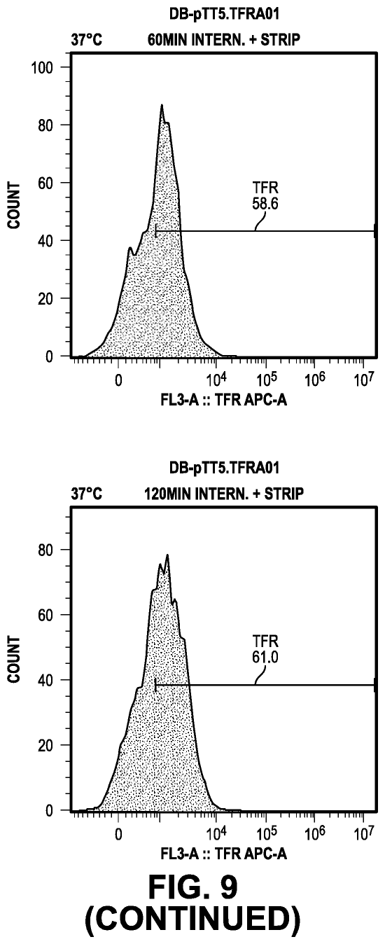

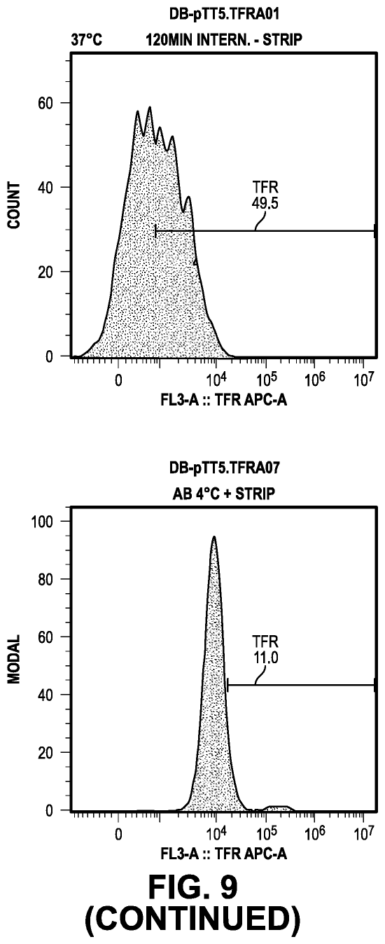

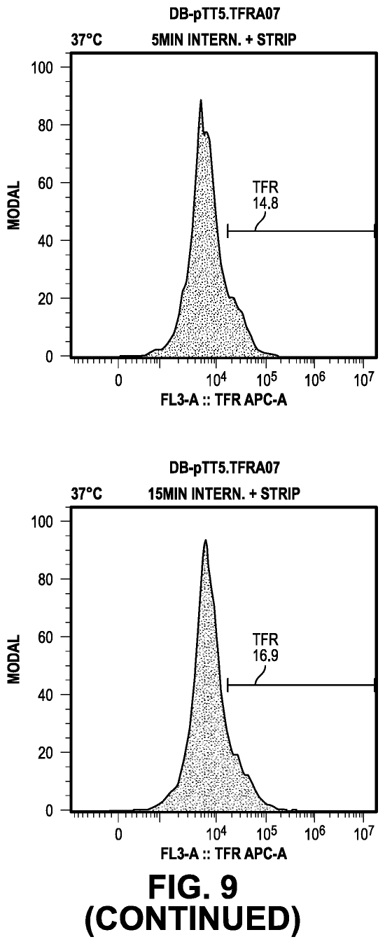

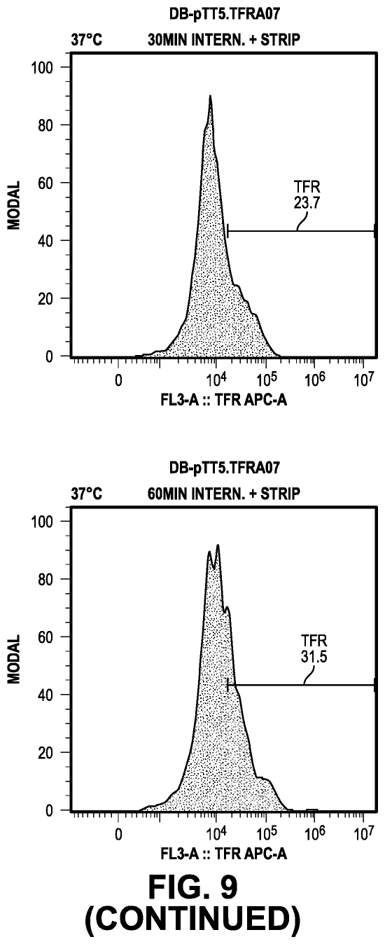

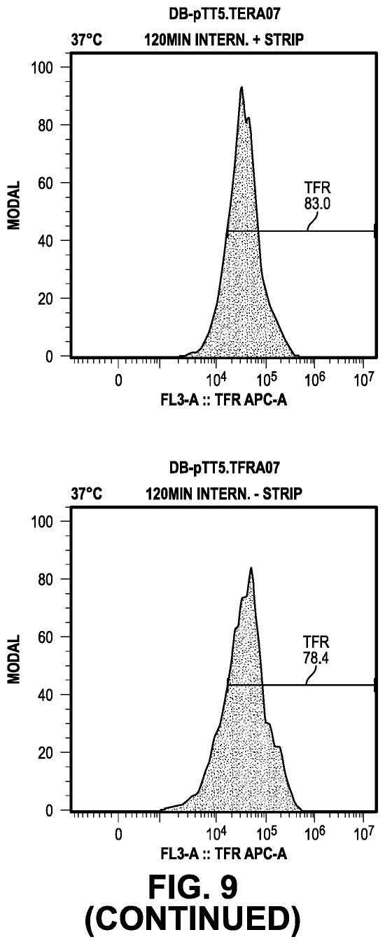

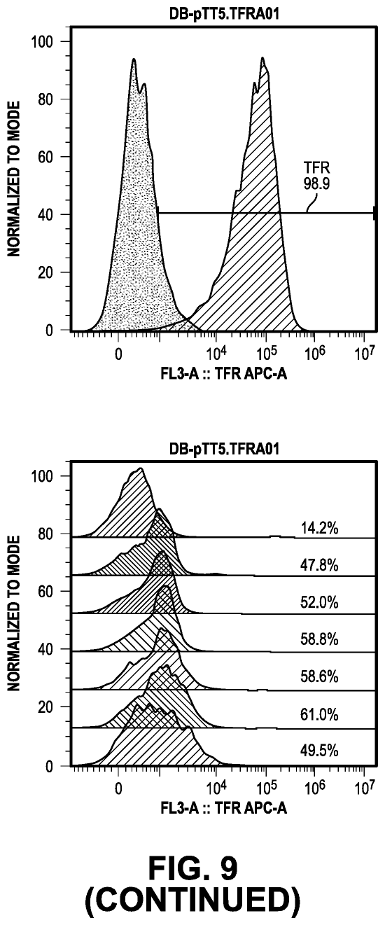

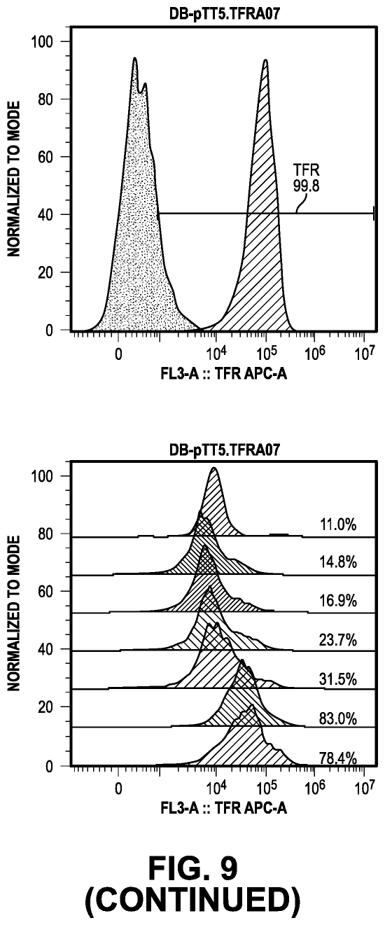

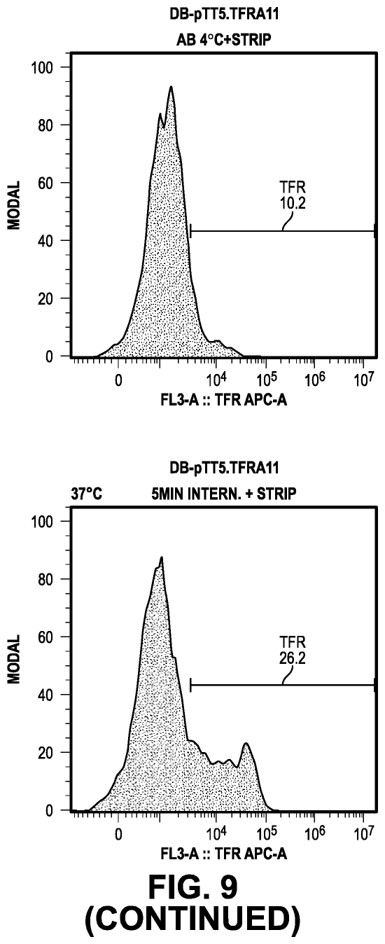

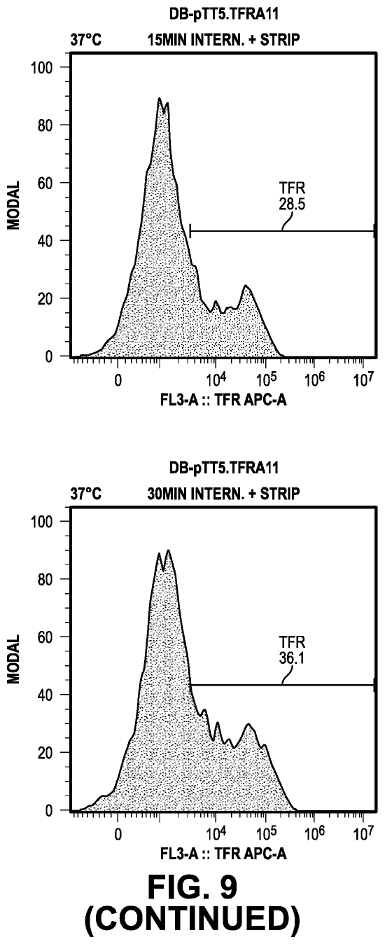

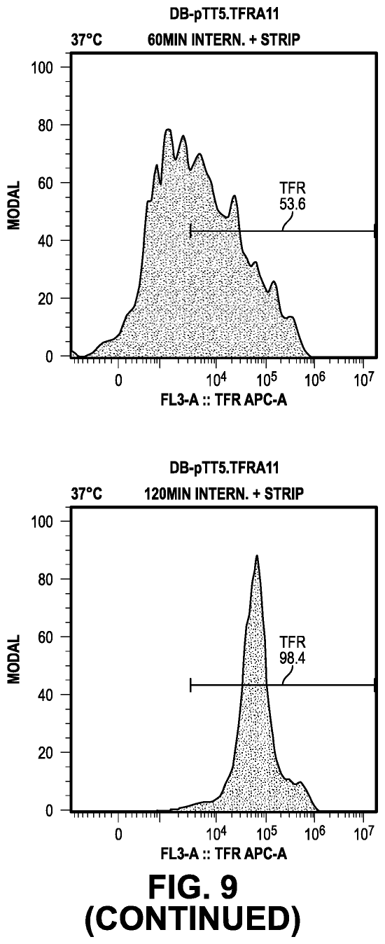

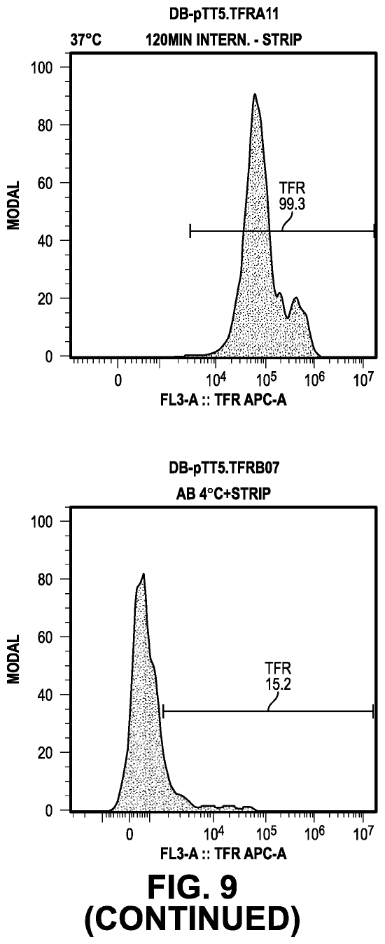

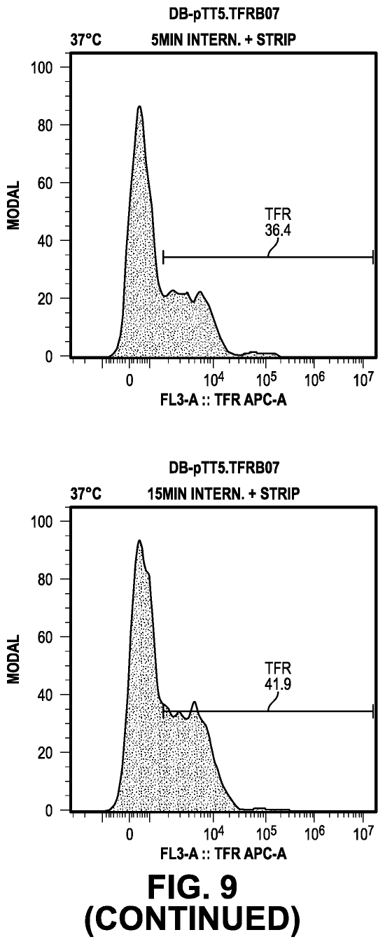

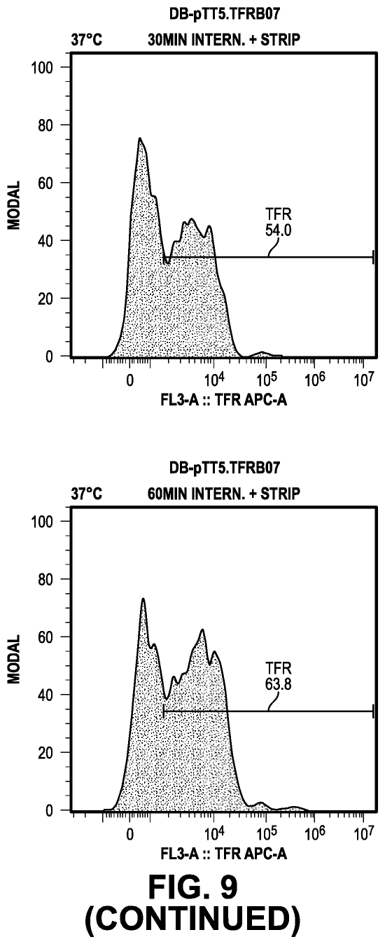

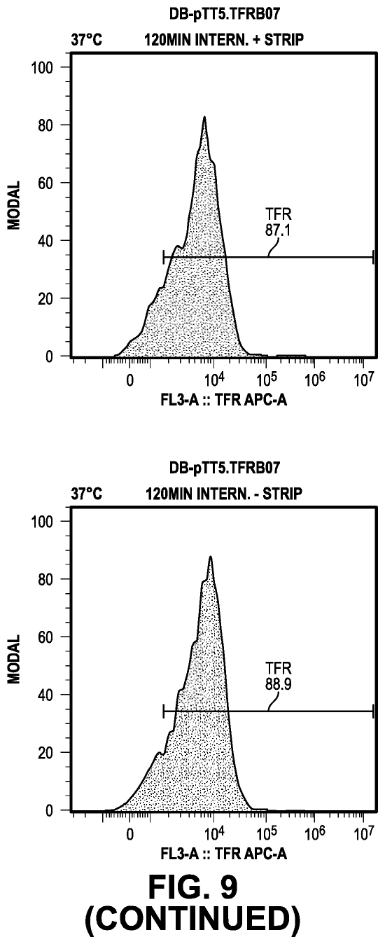

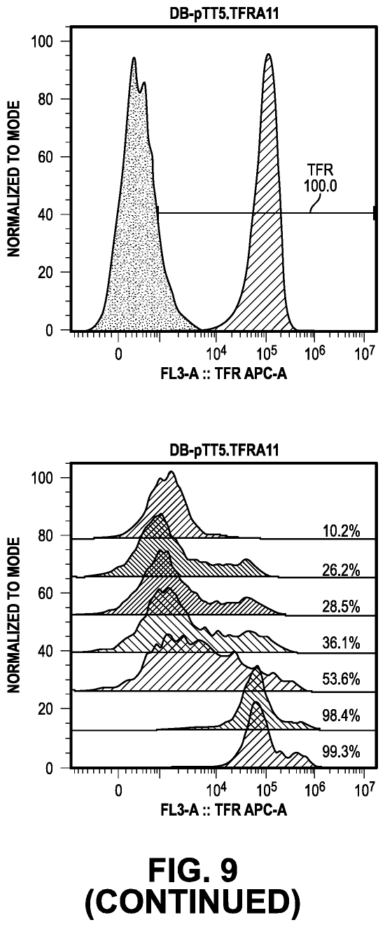

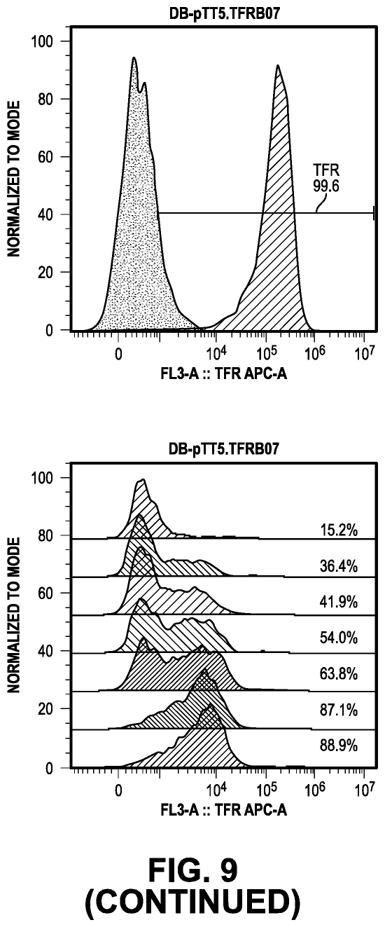

The results of this assay are provided in FIG. 9. In this cell line that expresses TFR, the data shows the anti-TFR light chains that were tested induced TFR-dependent internalization. Since the heavy chain of this antibody was a non-target specific epitope, it does not bind these cells.

### Example 3: Binding Affinity Assessment

This example describes the analysis of the binding affinity and dissociation constants of the interaction of the antibodies with TfR using surface plasmon resonance (SPR) or an ELISA.

BIAcore Surface Plasmon Resonance (SPR) Analysis of Anti-TfR Antibodies

SPR is performed on a Biacore 3000 System (BIAcore, Piscataway, N.J.) using CM5 sensor chip. CM5 chip matrix consists of a carboxymethylated dextran covalently attached to a gold surface. All measurements were performed at 25° C.

Neutravidin (Sigma, St. Louis, Mo.) is immobilized on a CM5 sensor chip (flow cells 1 to 4) by the amine-coupling protocol, at a level of 5000-10000 response units (RUs). The amine coupling protocol includes activation of the dextran matrix on the sensor chip surface with a 1:1 mixture of 0.4 M 1-ethyl-3-(3-dimethylaminopropyl carbodiimide (EDC) and 0.1 M N-hydroxysuccinimide (NHS), followed by injection of neutravidin in 10 mM sodium acetate buffer, pH 4. After neutravidin immobilization, the subsequent steps were carried out in HBS-EP buffer (10 mM HEPES, pH 7.4, 150 mM NaCl, 3 mM EDTA, and 0.005% surfactant P20).

Biotinylated TfR is immobilized in flow cells 2-4 (one species per flow cell) by injecting individual TfR at a concentration of 200 μg/ml at a flow rate of 5 μl/min, for 20 minutes (min).

After biotin-TFR analogs are captured on the chip, anti-TfR Ab is injected into flow cells 1-4 at the concentration of 24 μg/ml in HBS-EP buffer at the flow rate of 50 l/min, for 3 min. After 3 minutes, injection is stopped and dissociation is followed for 6 min. Regeneration is performed by injecting 10 mM glycine HCl, pH 1.5 at a flow rate of 10 μl/min for 1 min.

Resonance signals are corrected for non-specific binding by subtracting the signal of the control flow cell (cell 1) and is analyzed using BIAevaluation 4.1 software (Biacore).

The affinity and dissociation constants (Kds) can be determined.

ELISA

Binding affinities of the described antibodies can be measured using, for example, a competitive ELISA assay protocol. EC₅₀, or the concentration of antibody that gives half-maximal binding, is determined by direct and saturable binding of an antibody dilution series to both target antigen (TfR) and a non-specific control protein. An estimate of affinity is interpreted from one-half the concentration at which the antibody binding first achieves saturation. The assay can be done in a high-throughput manner using 3-5 fold dilutions of antibody of a stock antibody concentration in binding buffer. Since most antibodies will, at sufficiently high concentrations, begin to bind non-specifically to non-target proteins, this assay enables determination of an antibody that provides maximal specificity to non-specific signals when conducted using the appropriate non-specific control.

A determination of affinity can be obtained through direct binding of antibody to immobilized target protein in the presence of series of concentrations of soluble target antigen (TfR) in a competitive ELISA format. The quantitation of antibody EC₅₀ is required to establish an accurate sub-saturating concentration of antibody for conducting the competitive binding experiment. The multipoint competitive ELISA then evaluates binding of the sub-saturating antibody concentration to immobilized antigen competed by pre-incubation with serial dilutions of antigen (TfR) in solution to produce an inhibition curve from which the IC₅₀ value can be determined.

### Example 4: Transgenic Alzheimer's Disease Mouse Model

Animal models have been developed to test novel drugs. A standard model for Alzheimer's disease (AD) is transgenic mice that express human mutated genes for amyloid production that are known to induce Alzheimer's disease in humans. These APP/PS1 transgenic mice recapitulate some of the symptoms of Alzheimer's disease such as the aggregation of amyloid in the brain, memory loss and the loss of synapses (Radde et al., 2006, EMBO Rep., 7:940-647).

The APP/PS1 mice express human Swedish mutated APP and human mutated presenilin-1 which induce Alzheimer's disease in humans. Five-six (5-6) animals per group are tested.

Anti-TfR antibodies are administered at a dose of from about 5 nmol/kg to about 30 nmol/kg body weight. The antibodies are administered by a once-daily intraperitoneal injection for 8 weeks. Saline injections and/or isotype control antibody are administered as a control.

Brain Tissue Analysis for Amyloid Plaques and Synapse Numbers in AD Mice

Animals (5 per group) are perfused transcardially with 30 ml of ice-cold PBS and 30 ml of ice-cold 4% paraformaldehyde to postfix the brain. The brains are removed and placed in fresh 30% sucrose solution in PBS to cryoprotect tissue and cut at a thickness of 40 μm on a cryostat. Sections are chosen according to stereological rules, with the first section taken at random and every 5th section afterwards. Between 7 and 13 sections are analyzed per brain.

Immunostaining techniques are used to assess the neuronal plaque load (anti beta-amyloid rabbit polyclonal antibody (1:200, rabbit polyclonal-Invitrogen 71-5800) and of synaptophysin (polyclonal rabbit anti-synaptophysin primary antibody, 1:2000, Abcam, Cambridge, UK) to measure the number of synapses in the cortex. Brain samples are first exposed to 99% formic acid for 7 minutes and then washed 3 times for 10 minutes in TBS. Pre-treatment with 99% formic acid is known to drastically increase the detection of beta-amyloid in formalin-fixed brain samples. Then samples are incubated in 0.3% H₂O₂ (Sigma Aldrich; Cat.-No.: 516813) in TBS for 30 minutes on the shaker to deplete endogenous peroxides activity, and are 3 times washed in TBS for 10 minutes afterwards.

To increase permeability of membranes in the brain tissue, samples are exposed to 0.3% Triton X-100 (Sigma Aldrich; Cat.-No.: 516813) in TBS for 10 minutes on the shaker. Unspecific proteinophilic binding in the tissue is saturated by incubating the samples with 5% goat serum (Gibco; Cat.-No.: 16210-064) in TBS for 30 minutes on the shaker.

A binding agent is added and incubated overnight on the shaker at 4° C. The binding agent is used in a final dilution of 1 to 250 in TBS containing 2% goat serum and 10% Triton X-100. Afterwards, the samples are washed with TBS 3 times for 10 minutes and a secondary antibody was added for 90 minutes on the shaker at 4° C. The secondary antibody is a biotinylated anti-rabbit IgG raised in goat (Vectastain ABC Kit, Rabbit IgG; Vector Laboratories; Cat.-No.: PK-6101), and is used in a final dilution of 1 to 60 in TBS containing 1% goat serum and 10% Triton-X-100.

After washing 3 times for 10 minutes in TBS, samples are incubated in TBS containing 3% avidin solution (Vectastain ABC Kit, Rabbit IgG; Vector Laboratories; Cat.-No.: PK-6101) and 3% biotinylated horseradish peroxidase solution (Vectastain ABC Kit, Rabbit IgG; Vector Laboratories; Cat.-No.: PK-6101) for 90 minutes at 4° C. on the shaker. The samples are then washed 3 times in TBS for 10 minutes and then stained by adding phosphate-buffered saline containing 3% SG blue solution (SG Blue Peroxidase Kit; Vector Laboratories; Cat.-No.: SK-4700) and 3% H₂O₂ solution for 5 minutes and then again washed 3 times for 10 minutes with phosphate-buffered saline.

To enhance staining, samples are incubated with ddH₂O containing 0.5% CuSO₄ (w/w) for 5 minutes. After washing 3 times for 10 minutes in ddH₂O, the brain slices are put onto silane-coated glass slides with a fine brush where they dry overnight. Finally, the slides are cover slipped with an aquatic mounting medium (VectaMountAQ Mounting Medium; Vector Laboratories, Cat.-No.: H-5501). Samples from a 17 month old non-transgenic littermate processed together with the experimental samples were used as controls.

Sections are photographed under a microscope (Zeiss, Germany), randomized unbiased dissectors are overlain on to the brain section images and analyzed using a Multi threshold plug-in with the software Image J (NIH, USA). Data were analyzed using a one-way ANOVA with post-hoc Bonferroni tests.

### Example 5: Parkinson Mouse Model

A standard model to induce Parkinson-like symptoms in mice is the injection of the chemical, MPTP (Li et al., 2009). This chemical impairs or kills neurons in the brain that produce dopamine. The mice develop motor impairments, and the dopaminergic neurons in the brain are reduced in number and function. The enzyme tyrosine hydroxylase (TH) is required to synthetize dopamine. A loss of TH signifies a loss of dopamine production (Harkavyi et al., 2008, Journal of Neuroinflammation, 5:19).

Adult male C57BL/6 mice are given the dopaminergic toxin MPTP (20 mg/kg in 0.1 mL of PBS intraperitoneally (i.p.) at 2-h intervals of 4 doses MPTP; Sigma) or vehicle (PBS). This treatment selectively affects dopaminergic neurons and induces Parkinson-like symptoms in mice. One group does not receive MPTP as a non-lesioned control.

The dose of binding agent is tested at from about 5 nmol/kg to about 30 nmol/kg body weight. The binding agent is administered by a once-daily intraperitoneal injection for 8 weeks. Saline injections are administered as a control. Animals are tested in groups of 5 or 6 animals.

Rotarod Motor Control Test

The rotarod consists of a rotating pole that accelerates over time. Mice are placed on the rod, and motor skills are tested by accelerating the rotation. As the rotation increases, animals lose grip and fall on a cushion located below the rod. Mice are given three trials with 45 min inter-trial intervals on each of 2 consecutive days for 3 weeks. Each animal's endurance time is recorded, and the average is calculated. Data are analyzed using a one-way ANOVA with post-hoc Bonferroni tests.

Open-Field Motor Activity Task

Mice receive a session of 5 min in the empty open-field (58 cm in diameter; 35 cm high walls) with painted grey walls and grey floor. The movements and speed are tracked with a computerized video capture system and analysis software (Biosignals Inc., New York, USA). Motor activity is recorded by total path length and travel speed. Data are analyzed using two-way ANOVA with post-hoc tests.

Immunohistochemistry for Tyrosine Hydroxylase in the Substantia Nigra Pars Compacta and the Striatum

Animals are analyzed for expression of tyrosine hydroxylase (TH), a marker for dopamine production. Coronal brain sections (20 μm) from striatum (bregma 11 to 10.2) and SNpc (bregma 24.80 to 26.04) were analyzed by immunohistochemistry using antibodies recognizing TH.

Sections are cut on a cryostat and post-fixed in 4% paraformaldehyde, washed in PBS, treated with 0.3% H₂O₂ in methanol for 20 min, and washed again. Incubation with TH (1:800) antibody is at 48° C. overnight in PBS with 0.1% Tween and 10% goat serum. Sections are incubated for 1 hr at room temperature with biotinylated secondary antibody diluted in 0.1% PBS-Tween. DAB staining was performed according to the Vectastain ABC kit instructions (Vector Laboratories). For each animal, three tissue sections from one level of striatum was stained and analyzed for TH-positive fiber innervation. To achieve a TH cell count representative of the whole SN, each animal was analyzed at four and three rostrocaudal levels (bregma −4.80 to −6.04). Two tissue sections from each level were quantified by immunohistochemistry. Data are analyzed using a one-way ANOVA with post-hoc Bonferroni tests.

### Example 6: Mouse Model of Ovarian Cancer

To determine the ability of an antibody, antibody fragment, or binding agent to treat ovarian cancer, an ovarian cancer cell line can be used in SCID or nude mice. Briefly, ovarian cancer cells can be implanted into SCID or nude mice to generate ovarian tumors.

Groups of mice bearing established tumors can be treated by intravenous (i.v.) administration of escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The treatment can be performed 1-3 times per week for 4 weeks. The mice can be monitored, and tumor growth can be measured 2 to 3 times per week. Tumor growth can be measured as weight or volume. Mice receiving an antibody, antibody fragment, or binding agent can have less tumor growth than control mice receiving saline or control IgG. Tumor growth in mice receiving an antibody, antibody fragment, or binding agent can be at least about 5%, 10%, 15%, 20%, 25%, 30%, 35%, 40%, 45%, 50%, or more less than tumor growth in control mice.

### Example 7: Mouse Model of Kidney Cancer

To determine the ability of an antibody, antibody fragment, or binding agent to treat kidney cancer, a kidney cancer cell line can be used in SCID or nude mice. Briefly, kidney cancer cells can be implanted into SCID or nude mice to generate kidney tumors.

Groups of mice bearing established tumors can be treated by intravenous administration of escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The treatment can be performed 1-3 times per week for 4 weeks. The mice can be monitored, and tumor growth can be measured 2 to 3 times per week. Tumor growth can be measured as weight or volume. Mice receiving an antibody, antibody fragment, or binding agent can have less tumor growth than control mice receiving saline or control IgG. Tumor growth in mice receiving an antibody, antibody fragment, or binding agent can be at least about 5%, 10%, 15%, 20%, 25%, 30%, 35%, 40%, 45%, 50%, or more less than tumor growth in control mice.

### Example 8: Mouse Model of Myeloma

To determine the ability of an antibody, antibody fragment, or binding agent to treat myeloma, a myeloma cell line is used in SCID or nude mice. Briefly, myeloma cancer cells are implanted into SCID or nude mice to generate myeloma tumors.

Groups of mice bearing established tumors can be treated by intravenous administration of escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The treatment can be performed 1-3 times per week for 4 weeks. The mice can be monitored, and tumor growth can be measured 2 to 3 times per week. Tumor growth can be measured as weight or volume. Mice receiving an antibody, antibody fragment, or binding agent can have less tumor growth than control mice receiving saline or control IgG. Tumor growth in mice receiving an antibody, antibody fragment, or binding agent can be at least about 5%, 10%, 15%, 20%, 25%, 30%, 35%, 40%, 45%, 50%, or more less than tumor growth in control mice.

### Example 9: Mouse Model of Sarcoma

To determine the ability of an antibody, antibody fragment, or binding agent to treat sarcoma, a sarcoma cell line is used in SCID or nude mice. Briefly, sarcoma cancer cells are implanted into SCID or nude mice to generate sarcoma tumors.

Groups of mice bearing established tumors can be treated by intravenous administration of escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The treatment can be performed 1-3 times per week for 4 weeks. The mice can be monitored, and tumor growth can be measured 2 to 3 times per week. Tumor growth can be measured as weight or volume. Mice receiving an antibody, antibody fragment, or binding agent can have less tumor growth than control mice receiving saline or control IgG. Tumor growth in mice receiving an antibody, antibody fragment, or binding agent can be at least about 5%, 10%, 15%, 20%, 25%, 30%, 35%, 40%, 45%, 50%, or more less than tumor growth in control mice.

### Example 10: Mouse Model of Breast Cancer

To determine the ability of an antibody, antibody fragment, or binding agent to treat breast cancer, a breast cancer cell line is used in SCID or nude mice. Briefly, breast cancer cells can be implanted into SCID or nude mice to generate breast tumors.

Groups of mice bearing established tumors can be treated by intravenous administration of escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The treatment can be performed 1-3 times per week for 4 weeks. The mice can be monitored, and tumor growth can be measured 2 to 3 times per week. Tumor growth can be measured as weight or volume. Mice receiving an antibody, antibody fragment, or binding agent can have less tumor growth than control mice receiving saline or control IgG. Tumor growth in mice receiving an antibody, antibody fragment, or binding agent can be at least about 5%, 10%, 15%, 20%, 25%, 30%, 35%, 40%, 45%, 50%, or more less than tumor growth in control mice.

### Example 11: Systemic Toxicology in Cynomolgus Monkeys

Cynomolgus monkeys are utilized in a study to address the systemic toxicology of a binding agent. Briefly, monkeys are dosed weekly for three weeks with 10.0 mg/kg, 30.0 mg/kg, or 100.0 mg/kg of a binding agent. Placebo animals are dosed on the same schedule with an appropriate solution in the absence of the binding agent. The doses are administered as an intravenous bolus over 30 to 60 minutes and at least six animals are dosed at each dose level. Toxicology is assessed via one or more of the following indications: body weight measurements, basic physiologic clinical measurements, serial serum chemistry, hematologic evaluations, and histopathological evaluations.

### Example 12: Regional Toxicology Study in Cynomolgus Monkeys

Cynomolgus monkeys are utilized in a study to address the regional toxicology of binding agents. Briefly, monkeys are dosed by intravitreal injection weekly for six weeks with about 0.25 mg/kg, about 1.25 mg/kg, about 2.5 mg/kg, about 5 mg/kg, about 10 mg/kg, about 15 mg/kg, about 20 mg/kg, about 30 mg/kg, about 40 mg/kg, or about 50 mg/kg of a binding agent. Placebo animals are dosed on the same schedule with an appropriate solution in the absence of binding agent.

The doses are administered as intravitreal injections and at least six animals are dosed at each dose level. Toxicology is assessed via one or more of the following indications: body weight measurements, basic physiologic clinical measurements, serial serum chemistry, hematologic evaluations, and histopathological evaluations.

### Example 13: Animal Model for Crossing the Blood-Brain Barrier

An antibody, antibody fragment, or binding agent can be injected into an animal model such as a mouse model. Mice (4-6 mice per group) can receive about 1, 2, 5, 10, 20, 30, 40, 50, 100, 150, 200, 250, 300, 350, 400, 450, 500, 750, or 1000 mg/kg body weight. Control animals can be administered a control IgG or saline.

After 0.5, 1, 2, 3, 6, 12, 18, or 24 hours, mice can be sacrificed, and mice can be excised. Brains can be analyzed whole, or can be dissected into parts (e.g., cerebellum, hippocampus, brain stem, etc.). In some cases, cerebrospinal fluid can be collected via a cisterna magna puncture prior to sacrifice.

Brains or brain portions can be sectioned and fixed onto slides. Immunohistochemistry or immunofluorescence can be performed on brain tissues to determine the presence of the injected antibody.

Whole brain or brain portions can be homogenized, and the protein fraction can be extracted. Western blotting can be performed with the whole brain, brain portions, or CSF samples.

With either immunofluorescence, immunohistochemistry, or western blotting, antibodies, antibody fragments, or binding agents can be detected. In some cases, an optimum dose can be determined, such that at that dose, the highest proportion of injected antibody is able to cross the blood-brain barrier and be detected in brain tissue.

### Example 14: Mouse Model of Bell's Palsy

A mouse model of Bell's palsy, for example, a model induced by reactivation of herpes simplex virus 1, can be employed to determine the ability of an antibody, antibody fragment, or binding agent to treat Bell's palsy.

Mice which develop Bell's palsy can be administered escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

Symptoms of Bell's palsy in the mice can be monitored. For example, facial nerve paralysis can be monitored, in some cases by the blink reflex or vibrissae movement score. Facial nerve paralysis can improve in mice receiving an antibody, antibody fragment, or binding agent compared with control animals. Improvement of facial nerve paralysis can be at least 10%, 20%, 30%, 40%, 50%, or more improvement.

### Example 15: Mouse Model of Cerebral Palsy

A mouse model of cerebral palsy, for example, a model wherein newborn mice are exposed to hypoxic brain injury, can be employed to determine the ability of an antibody, antibody fragment, or binding agent to treat cerebral palsy.

Newborn, youth, adolescent, or adult mice which develop cerebral palsy can be administered escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

Symptoms of cerebral palsy in the mice can be monitored. For example, short-term memory is assessed via a step-down avoidance task, and step-down latency can be used as a measure of short-term memory. Short term memory can improve in mice receiving an antibody, antibody fragment, or binding agent compared with control animals, which can indicate an improvement in the cerebral palsy condition. Improvement of short-term memory can be at least 5%, 10%, 15%, 20%, 25%, 30%, or more.

### Example 16: Mouse Model of Epilepsy

A mouse model of epilepsy, for example, a model wherein a mouse is administered pilocarpine or kainic acid, can be employed to determine the ability of an antibody, antibody fragment, or binding agent to treat epilepsy.

Mice which develop epilepsy can be administered escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

Symptoms of epilepsy can be monitored, for example, by monitoring seizure frequency or severity, or using MRI to monitor hippocampal injury.

In some cases, epilepsy can improve in mice receiving an antibody, antibody fragment, or binding agent. Improvement can be measured as a reduction in frequency or severity of seizures compared with control mice. Treated mice can have at least a 5%, 10%, 15%, 20%, 25%, 35%, or more reduction in seizure frequency compared with control mice. In some cases, improvement can be measured as a reduction in hippocampal injury, or a slower progression of hippocampal injury compared with control mice. Treated mice can have at least a 5%, 10%, 15%, 20%, 25%, 35%, or more reduction of hippocampal injury compared with control mice. Treated mice can have at least a 5%, 10%, 15%, 20%, 25%, 35%, or more slowing of hippocampal injury compared with control mice.

### Example 17: Mouse Model of Motor Neurone Disease

A mouse model of motor neurone disease, for example, a model of amyotrophic lateral sclerosis, can be employed to determine the ability of an antibody, antibody fragment, or binding agent to treat motor neurone disease. Mice can have a superoxide dismutase (SOD1) mutation, a TAR DNA-binding protein 43 (TDP43) transgene or mutation, a TDP-43 heterozygous knockout, a TDP-43 overexpression, or other genetic defect leading to motor neurone disease.

Mice having motor neurone disease can be administered escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

Symptoms of motor neurone disease can be monitored, for example, by assessing the live expectancy of mice. Mice treated with an antibody, antibody fragment, or binding agent can improve the life expectancy of mice having motor neurone disease by at least 5%, 10%, 15%, 20%, 25%, 35%, or more compared with control animals.

### Example 18: Mouse Model of Multiple Sclerosis

A mouse model of multiple sclerosis, for example, experimental autoimmune encephalomyelitis (EAE) and Theiler's Murine Encephalitis Virus-Induced Demyelinating Disease (TMEV-IDD) models, can be employed to determine the ability of an antibody, antibody fragment, or binding agent to treat multiple sclerosis.

Mice having a multiple sclerosis model disease can be administered escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The disease can be monitored, for example, by assessing the development of new brain lesions after 1 month, 2 months, 3 months, 4 months, 5 months, or 6 months, for example, by MRI. Mice treated with an antibody, antibody fragment, or binding agent can have at least about 5%, 10%, 15%, 20%, 25%, 30%, 35%, 40%, 45%, 50%, 55%, 60%, 65%, 70%, 75%, 80%, 85%, 90%, or 95% fewer lesions at the end of the study period.

### Example 19: Mouse Model of Neurofitromatosis

A mouse model of neurofitromatosis, for example, a mouse with a mutation in Nf1, a Nf1 knockout, or a Nf1 transgene can be employed to determine the ability of an antibody, antibody fragment, or binding agent to treat neurofitromatosis.

Mice having neurofitromatosis can be administered escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The disease can be monitored, for example, by assessing or measuring tumorigenic effects in the mice. In some cases, mice treated with an antibody, antibody fragment, or binding agent can experience fewer or reduced tumorigenic effects compared with control animals.

### Example 20: Mouse Model of Stroke

A mouse model of stroke, for example, a cerebral ischemia-reperfusion model, can be employed to determine the ability of an antibody, antibody fragment, or binding agent to treat stroke.

Mice having experienced a stroke, or a model of stroke, can be administered escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The disease prognosis can be monitored, for example, by imaging (e.g., MRI, CT, or PET). Mice having been administered an antibody, antibody fragment, or binding agent can have a better prognosis than control animals.

### Example 21: Mouse Model of Sciatica

A mouse model of sciatica, such as a dorsal root ganglion compression model, can be employed to determine the ability of an antibody, antibody fragment, or binding agent to treat sciatica.

Mice having sciatica or a model of sciatica can be administered escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The sciatica can be monitored or assessed, for example, by analysis of the mouse's gait. Mice having been administered an antibody, antibody fragment, or binding agent can have gait improvement compared with control animals.

### Example 22: Mouse Model of Shingles

A mouse model of shingles, for example, a mouse having had a primary infection with varicella zoster virus followed by the establishment of latency in sensory ganglia, can be employed to determine the ability of an antibody, antibody fragment, or binding agent to treat shingles.

Mice having shingles can be administered escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The symptoms and duration of shingles can be monitored in the mouse. Mice having been administered an antibody, antibody fragment, or binding agent can have fewer symptoms, less severe symptoms, or a shorter duration of disease compared with control mice.

### Example 23: Mouse Model of Testicular Cancer

To determine the ability of an antibody, antibody fragment, or binding agent to treat testicular cancer, a testicular cancer mouse model can be employed. The mouse model can be a spontaneous model, or it can be a xenograft model in which a testicular cancer cell line can be used in SCID or nude mice.

Groups of mice bearing established tumors can be treated by intravenous administration of escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The treatment can be performed 1-3 times per week for 4 weeks. The mice can be monitored, and tumor growth can be measured 2 to 3 times per week. Tumor growth can be measured as weight or volume. Mice receiving an antibody, antibody fragment, or binding agent can have less tumor growth than control mice receiving saline or control IgG. Tumor growth in mice receiving an antibody, antibody fragment, or binding agent can be at least about 5%, 10%, 15%, 20%, 25%, 30%, 35%, 40%, 45%, 50%, or more less than tumor growth in control mice.

### Example 24: Mouse Model of Brain Tumor

A mouse model of a brain tumor can be used to determine the ability of an antibody, antibody fragment, or binding agent to treat breast cancer, a breast cancer cell line is used in a brain tumor model of mice. Mice can be SCID mice, immunodeficient mice, or nude mice. Some mouse models can be spontaneous tumor models, and some mouse models can be induced tumor models.

Models of brain tumor can include acoustic neuroma, astrocytoma, chordoma, CNS lymphoma, craniopharyngioma, brain stem glioma, ependymoma, mixed glioma, optic nerve glioma, subpendymoma, medulloblastoma, meningioma, metastatic brain tumor, glioblastoma multiforme, oligodendroglioma, pituitary tumor, primitive neurodectodermal tumor, schwannoma, juvenile pilocytic astrocytoma, pineal tumor, or rhabdoid tumor.

Briefly, groups of mice bearing established tumors can be treated by intravenous administration of escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The treatment can be performed 1-3 times per week for 4 weeks. The mice can be monitored, and tumor growth can be measured 2 to 3 times per week. Tumor growth can be measured as weight or volume. Mice receiving an antibody, antibody fragment, or binding agent can have less tumor growth than control mice receiving saline or control IgG. Tumor growth in mice receiving an antibody, antibody fragment, or binding agent can be at least about 5%, 10%, 15%, 20%, 25%, 30%, 35%, 40%, 45%, 50%, or more less than tumor growth in control mice.

In some cases, imaging studies such as MRI, PET, CT, or BLI can be performed to determine how much antibody crossed the blood-brain barrier or blood CSF barrier to enter the brain or CNS.

### Example 25: Mouse Model of Retinal Cancer

To determine the ability of an antibody, antibody fragment, or binding agent to treat retinal cancer, a retinal cancer mouse model can be employed. The mouse model can be a spontaneous model, or it can be a xenograft model in which a retinal cancer cell line can be used in SCID or nude mice.

Groups of mice bearing established tumors can be treated by intravenous administration of escalating doses (for example, starting at 1.8 mg/kg body weight) of an antibody, antibody fragment, or binding agent. In some cases, mice can receive up to 2, 5, 10, 20, 30, 40, 50, 60, 70, 80, 90, or 100 mg/kg body weight of an antibody, antibody fragment, or binding agent. Control animals can be administered a control IgG or saline.

The treatment can be performed 1-3 times per week for 4 weeks. The mice can be monitored, and tumor growth can be measured 2 to 3 times per week. Tumor growth can be measured as weight or volume. Mice receiving an antibody, antibody fragment, or binding agent can have less tumor growth than control mice receiving saline or control IgG. Tumor growth in mice receiving an antibody, antibody fragment, or binding agent can be at least about 5%, 10%, 15%, 20%, 25%, 30%, 35%, 40%, 45%, 50%, or more less than tumor growth in control mice.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-5. (canceled)

6. An antibody or an antigen-binding fragment that selectively binds to a transferrin receptor (TfR), that comprises a heavy chain variable region (VH) and a light chain variable region (VL), wherein the VL comprises:
(i) a complementarity determining region (CDR) 1 (CDR1) having an amino acid sequence that is at least 80%, identical to any one of SEQ ID NOS: 13-27 or an amino acid sequence of RASQTLYTNYLA (SEQ ID NO: 26); KSSRSVLRTSKNKNFLA (SEQ ID NO: 27); or X₁ASX₂X₃X₄X₅X₆X₇LX₈ (SEQ ID NO: 13), wherein X₁ comprises R or Q; X₂ comprises Q or R; X₃ comprises G, D, S or N; X₄ comprises I or V; X₅ comprises S, R, G, N or K; X₆ comprises R, K, S, G, or D; X₇ comprises N, W, Y, A, R, or K; and X₈ comprises A or N;
(ii) a CDR2 having an amino acid sequence that is at least 80%, identical to any one of SEQ ID NOS: 32-41 or an amino acid sequence of X₁X₂X₃X₄X₅ X₆X₇ (SEQ ID NO: 32), wherein X₁ comprises G, A, K, W, or S; X₂ comprises A or T; X₃ comprises F or S, X₄ comprises T, R, S, or N; X₅ comprises R or L; X₆ comprises R, Q, A, or E; and X₇ comprises S, N, or T; and
(iii) a CDR3 having an amino acid sequence that is at least 80%, identical to any one of SEQ ID NOS: 47-59 or an amino acid sequence of CQX₁X₂X₃X₄X₅PX₆TF (SEQ ID NO: 47), wherein X₁ comprises Q or K; X₂ comprises S, A, G, Y, H; X₃ comprises Y, N, F, K, G, or L; X₄ comprises K, S, or R; X₅ comprises T, F, Y, A, L, R, P, or S; and X₆ comprises Y, W, F, R, L, or I.

7-27. (canceled)

28. The antibody or the antigen-binding fragment of claim 6, wherein the VH comprises an amino acid sequence that is at least 80%, identical to SEQ ID NO: 87.

29. (canceled)

30. The antibody or the antigen-binding fragment of claim 6, wherein the antibody comprises a monoclonal antibody, a chimeric antibody, a human antibody, a bi-valent antibody, a multi-valent antibody, a maxibody, a humanized antibody, a deimmunized antibody, a humanized and deimmunized antibody, a mimetic thereof, a conjugate thereof, a fusion thereof, or a combination thereof.

31. The antibody or the antigen-binding fragment of claim 6, wherein the antigen-binding fragment is a Fab, a Fab′, a F(ab′)₂, a Fv, a scFv, a triabody, a tetrabody, a minibody, a bispecific F(ab′)₂, a trispecific F(ab′)₂, a diabody, a bispecific diabody, a single chain binding polypeptide, or a bispecific scFv.

32. The antibody or the antigen-binding fragment of claim 6, that comprises a binding affinity for the TfR of from about 1 nM to about 5 μM.

33. The antibody or the antigen-binding fragment of claim 6, that comprises a binding affinity for the TfR of from about 1 nM to about 500 nM, from about 50 nM to about 400 nM, from about 100 nM to about 300 nM, from about 150 nM to about 250 nM, or from about 175 nM to about 225 nM.

34. The antibody or the antigen-binding fragment of claim 6, that comprises a modified antibody or a modified antigen-binding fragment.

35. (canceled)

36. The antibody or the antigen-binding fragment of claim 6, that is an IgG, an IgA, an IgD, an IgE, or an IgM.

37. The antibody or the antigen-binding fragment of claim 34, comprising the modified antibody, wherein the modified antibody comprises a first polypeptide and a second polypeptide, each comprising a C_{H}3 antibody constant domain, wherein the first polypeptide and the second polypeptide meet at an engineered interface within the C_{H}3 domain, wherein the first polypeptide or the second polypeptide comprises a VH that selectively binds to a transferrin receptor, and the VH comprises a CDR3 that is encoded by the nucleic acid sequence of SEQ ID NO: 8, or a nucleic acid sequence that is at least 80%, identical to SEQ ID NO: 8.

38. (canceled)

39. (canceled)

40. (canceled)

41. The modified antibody of claim 37, wherein the VH comprises a CDR1 having an amino acid sequence that is at least 80% identical to SEQ ID NO: 2; a CDR2 having an amino acid sequence that is at least 80% identical to SEQ ID NO: 4; and a CDR3 having an amino acid sequence that is at least 80% identical to SEQ ID NO: 6.

42. (canceled)

43. (canceled)

44. (canceled)

45. (canceled)

46. The antibody or the antigen-binding fragment of claim 37, wherein the first polypeptide comprises an engineered protuberance in the interface of the first polypeptide within its C_{H}3 domain created by replacing at least one contact residue of the first polypeptide within its C_{H}3 domain, and wherein the second polypeptide comprises an engineered cavity in the interface of the second polypeptide within its C_{H}3 domain.

47. The antibody or the antigen-binding fragment of claim 46, wherein the engineered protuberance in the interface of the first polypeptide is positional in the engineered cavity of the second polypeptide so as to form a protuberance-into-cavity mutant pair.

48. The antibody or the antigen-binding fragment of claim 37, wherein the engineered interface within the C_{H}3 domain comprises at least two protuberance-into-cavity mutant pairs.

49. The antibody or the antigen-binding fragment of claim 48, wherein the at least two protuberance-into-cavity mutant pairs are created by creating at least one protuberance and at least one cavity on the first polypeptide and creating at least one cavity and at least one protuberance on the second polypeptide.

50. The antibody or the antigen-binding fragment of claim 48, wherein the at least two protuberance-into-cavity mutant pairs are created by creating more than one protuberance on the first polypeptide and creating more than one cavity on the second polypeptide.

51. (canceled)

52. (canceled)

53. (canceled)

54. (canceled)

55. The antibody or the antigen-binding fragment of claim 37, that comprises a bispecific modified antibody or a bispecific modified antigen-binding fragment, a trispecific modified antibody or a trispecific modified antigen-binding fragment, or a tetraspecific modified antibody or a tetraspecific modified antigen-binding fragment.

56. (canceled)

57. (canceled)

58. (canceled)

59. The antibody or the antigen-binding fragment of claim 6, that is capable of crossing the blood-brain barrier.

60. The antibody or the antigen-binding fragment of claim 6, that further binds to one or more brain agents.

61. The antibody or the antigen-binding fragment of claim 60, that further comprises a linker.

62. (canceled)

63. (canceled)

64. The antibody or the antigen-binding fragment of claim 60, that further comprises a fusion protein, wherein the fusion protein comprises another protein bound to the C-terminal side of the binding agent.

65. The antibody or the antigen-binding fragment of claim 64, wherein the fusion protein comprises a lysosomal enzyme.

66. (canceled)

67. (canceled)

68. The antibody or the antigen-binding fragment of claim 60, that is capable of crossing the blood-brain barrier.

69. (canceled)

70. (canceled)

71. (canceled)

72. (canceled)

73. (canceled)

74. (canceled)

75. A pharmaceutical composition or a medicament that comprises the antibody or the antigen-binding fragment of claim 6 and one or more pharmaceutically acceptable excipients.

76. (canceled)

77. (canceled)

78. (canceled)

79. (canceled)

80. (canceled)

81. A method of treating a neurological disease, a central nervous system (CNS) disease, a cancer or metastasis thereof, a neuroendocrine disease, a metabolic disease, or a combination thereof, in a subject in need thereof, comprising administering to the subject the antibody or the antigen-binding fragment of claim 6, whereby the neurological disease, the central nervous system (CNS) disease, the cancer or metastasis thereof, the neuroendocrine disease, the metabolic disease, or the combination thereof, is treated.

82. (canceled)

83. (canceled)

84. (canceled)

85. (canceled)

86. (canceled)

87. (canceled)

88. (canceled)

89. (canceled)

90. The method of claim 81, wherein the neurological disorder, the central nervous system (CNS) disease, or the combination thereof, is selected from the group consisting of Bell's palsy, cerebral palsy, epilepsy, Alzheimer's disease, motor neurone disease (MND), multiple sclerosis (MS), a neurofibromatosis, Parkinson's disease, stroke, sciatica, and shingles.

91. The antibody or the antigen-binding fragment of claim 6, wherein the VL comprises:
(i) a VL CDR1 having an amino acid sequence of SEQ ID NO: 26; a VL CDR2 having an amino acid sequence of SEQ ID NO: 33; and a VL CDR3 having an amino acid sequence of SEQ ID NO: 48;
(ii) a VL CDR1 having an amino acid sequence of SEQ ID NO: 15; a VL CDR2 having an amino acid sequence of SEQ ID NO: 34; and a VL CDR3 having an amino acid sequence of SEQ ID NO: 49;
(iii) a VL CDR1 having an amino acid sequence of SEQ ID NO: 14; a VL CDR2 having an amino acid sequence of SEQ ID NO: 35; and a VL CDR3 having an amino acid sequence of SEQ ID NO: 50;
(iv) a VL CDR1 having an amino acid sequence of SEQ ID NO: 16; a VL CDR2 having an amino acid sequence of SEQ ID NO: 36; and a VL CDR3 having an amino acid sequence of SEQ ID NO: 51;
(v) a VL CDR1 having an amino acid sequence of SEQ ID NO: 17; a VL CDR2 having an amino acid sequence of SEQ ID NO: 35; and a VL CDR3 having an amino acid sequence of SEQ ID NO: 50;
(vi) a VL CDR1 having an amino acid sequence of SEQ ID NO: 18; a VL CDR2 having an amino acid sequence of SEQ ID NO: 37; and a VL CDR3 having an amino acid sequence of SEQ ID NO: 50;
(vii) a VL CDR1 having an amino acid sequence of SEQ ID NO: 19; a VL CDR2 having an amino acid sequence of SEQ ID NO: 33; and a VL CDR3 having an amino acid sequence of SEQ ID NO: 5;
(viii) a VL CDR1 having an amino acid sequence of SEQ ID NO: 27; a VL CDR2 having an amino acid sequence of SEQ ID NO: 38; and a VL CDR3 having an amino acid sequence of SEQ ID NO: 53;
(ix) a VL CDR1 having an amino acid sequence of SEQ ID NO: 20; a VL CDR2 having an amino acid sequence of SEQ ID NO: 39; and a VL CDR3 having an amino acid sequence of SEQ ID NO: 54;
(x) a VL CDR1 having an amino acid sequence of SEQ ID NO: 21; a VL CDR2 having an amino acid sequence of SEQ ID NO: 40; and a VL CDR3 having an amino acid sequence of SEQ ID NO: 55;
(xi) a VL CDR1 having an amino acid sequence of SEQ ID NO: 22; a VL CDR2 having an amino acid sequence of SEQ ID NO: 41; and a VL CDR3 having an amino acid sequence of SEQ ID NO: 56;
(xii) a VL CDR1 having an amino acid sequence of SEQ ID NO: 23; a VL CDR2 having an amino acid sequence of SEQ ID NO: 33; and a VL CDR3 having an amino acid sequence of SEQ ID NO: 57;
(xiii) a VL CDR1 having an amino acid sequence of SEQ ID NO: 24; a VL CDR2 having an amino acid sequence of SEQ ID NO: 42; and a VL CDR3 having an amino acid sequence of SEQ ID NO: 58; and
(xiv) a VL CDR1 having an amino acid sequence of SEQ ID NO: 25; a VL CDR2 having an amino acid sequence of SEQ ID NO: 33; and a VL CDR3 having an amino acid sequence of SEQ ID NO: 59.


SEQUENCE LISTING (117 sequences)

SEQ ID NO: 1 - protein, 26 aa, Artificial Sequence
  Description of Artificial Sequence: Synthetic peptide
EVQLLESGGGLVQPGGSLRLSCAASG

SEQ ID NO: 2 - protein, 9 aa, Artificial Sequence
  Description of Artificial Sequence: Synthetic peptide
FTFSSYAMS

SEQ ID NO: 3 - protein, 13 aa, Artificial Sequence
  Description of Artificial Sequence: Synthetic peptide
WVRQAPGKGLEWV

SEQ ID NO: 4 - protein, 13 aa, Artificial Sequence
  Description of Artificial Sequence: Synthetic peptide
SAISGSGGSTYYA

SEQ ID NO: 5 - protein, 34 aa, Artificial Sequence
  Description of Artificial Sequence: Synthetic polypeptide
DSVKGRFTISRDNSKNTLYLQMNSLRAEDTAVYY

SEQ ID NO: 6 - protein, 16 aa, Artificial Sequence
  Description of Artificial Sequence: Synthetic peptide
CAKGGRDGYKGYFDYW

SEQ ID NO: 7 - protein, 10 aa, Artificial Sequence
  Description of Artificial Sequence: Synthetic peptide
GQGTLVTVSS

SEQ ID NO: 8 - DNA, 48 bp, Artificial Sequence
  Description of Artificial Sequence: Synthetic oligonucleotide
tgtgcgaaaggcgggcgcgatgggtataagggctactttgactactgg

SEQ ID NO: 9 - protein, 23 aa, Artificial Sequence
  Description of Artificial Sequence: Synthetic peptide
  MOD_RES (1)..(1): D or E
  MOD_RES (3)..(3): Q or V
  MOD_RES (9)..(9): S, D, or A
  MOD_RES (10)..(10): S or T
  MOD_RES (12)..(12): S or A
  MOD_RES (13)..(13): A or V
  MOD_RES (15)..(15): L or P
  MOD_RES (17)..(17): D or E
  MOD_RES (19)..(19): V or A
  MOD_RES (21)..(21): I or L
  MOD_RES (22)..(22): T, N, or S
XIXMTQSPXXLXXSXGXRXTXXC

SEQ ID NO: 10 - protein, 23 aa, Artificial Sequence
  Description of Artificial Sequence: Synthetic peptide
DIQMTQSPSSLSASVGDRVTITC

SEQ ID NO: 11 - protein, 23 aa, Artificial Sequence
  Description of Artificial Sequence: Synthetic peptide
DIVMTQSPDSLAVSLGERATINC

SEQ ID NO: 12 - protein, 23 aa, Artificial Sequence
  Description of Artificial Sequence: Synthetic peptide
EIVMTQSPATLSVSPGERATLSC

SEQ ID NO: 13 - protein, 11 aa, Artificial Sequence
  Description of Artificial Sequence: Synthetic peptide
  MOD_RES (1)..(1): R or Q
  MOD_RES (4)..(4): Q or R
  MOD_RES (5)..(5): G, D, S or N
  MOD_RES (6)..(6): I or V
  MOD_RES (7)..(7): S, R, G, N or K
  MOD_RES (8)..(8): R, K, S, G, or D
  MOD_RES (9)..(9): N, W, Y, A, R, or K
  MOD_RES (11)..(11): A or N
XASXXXXXXLX

SEQ ID NO: 14 - protein, 11 aa, Artificial Sequence
  Description of Artificial Sequence: Synthetic peptide
RASRGISRWLA

SEQ ID NO: 15 - protein, 11 aa, Artificial Sequence
  Description of Artificial Sequence: Synthetic peptide
QASQDIIDSLN

SEQ ID NO: 16 - protein, 11 aa, Artificial Sequence
  Description of Artificial Sequence: Synthetic peptide
RASQDIRRYLA

SEQ ID NO: 17 - protein, 11 aa, Artificial Sequence
  Description of Artificial Sequence: Synthetic peptide
RASRGVSKWLA

SEQ ID NO: 18 - protein, 11 aa, Artificial Sequence
  Description of Artificial Sequence: Synthetic peptide
RASRGVSSWLA

SEQ ID NO: 19 - protein, 11 aa, Artificial Sequence
  Description of Artificial Sequence: Synthetic peptide
RASRSVGGALA

SEQ ID NO: 20 - protein, 11 aa, Artificial Sequence
  Description of Artificial Sequence: Synthetic peptide
RASQSIRRYLN

SEQ ID NO: 21 - protein, 11 aa, Artificial Sequence
  Description of Artificial Sequence: Synthetic peptide
RASQNINKNLN

SEQ ID NO: 22 - protein, 11 aa, Artificial Sequence
  Description of Artificial Sequence: Synthetic peptide
RASQNIGSRLN

SEQ ID NO: 23 - protein, 11 aa, Artificial Sequence
  Description of Artificial Sequence: Synthetic peptide
RASRSISDYLA

SEQ ID NO: 24 - protein, 11 aa, Artificial Sequence
  Description of Artificial Sequence: Synthetic peptide
RASQNIKRYLN

SEQ ID NO: 25 - protein, 11 aa, Artificial Sequence
  Description of Artificial Sequence: Synthetic peptide
RASQSVRRKLA

SEQ ID NO: 26 - protein, 12 aa, Artificial Sequence
  Description of Artificial Sequence: Synthetic peptide
RASQTLYTNYLA

SEQ ID NO: 27 - protein, 17 aa, Artificial Sequence
  Description of Artificial Sequence: Synthetic peptide
KSSRSVLRTSKNKNFLA

SEQ ID NO: 28 - protein, 15 aa, Artificial Sequence
  Description of Artificial Sequence: Synthetic peptide
  MOD_RES (8)..(8): Q or K
  MOD_RES (9)..(9): A or P
  MOD_RES (11)..(11): R or K
WYQQKPGXXPXLLIY

SEQ ID NO: 29 - protein, 15 aa, Artificial Sequence
  Description of Artificial Sequence: Synthetic peptide
WYQQKPGQAPRLLIY

SEQ ID NO: 30 - protein, 15 aa, Artificial Sequence
  Description of Artificial Sequence: Synthetic peptide
WYQQKPGKAPKLLIY

SEQ ID NO: 31 - protein, 15 aa, Artificial Sequence
  Description of Artificial Sequence: Synthetic peptide
WYQQKPGQPPKLLIY

SEQ ID NO: 32 - protein, 7 aa, Artificial Sequence
  Description of Artificial Sequence: Synthetic peptide
  MOD_RES (1)..(1): G, A, K, W, or S
  MOD_RES (2)..(2): A or T
  MOD_RES (3)..(3): F or S
  MOD_RES (4)..(4): T, R, S, or N
  MOD_RES (5)..(5): R or L
  MOD_RES (6)..(6): R, Q, A, or E
  MOD_RES (7)..(7): S, N, or T
XXXXXXX

SEQ ID NO: 33 - protein, 7 aa, Artificial Sequence
  Description of Artificial Sequence: Synthetic peptide
GASTRAT

SEQ ID NO: 34 - protein, 7 aa, Artificial Sequence
  Description of Artificial Sequence: Synthetic peptide
AAFRLRS

SEQ ID NO: 35 - protein, 7 aa, Artificial Sequence
  Description of Artificial Sequence: Synthetic peptide
AASSLQS

SEQ ID NO: 36 - protein, 7 aa, Artificial Sequence
  Description of Artificial Sequence: Synthetic peptide
KASRLQS

SEQ ID NO: 37 - protein, 7 aa, Artificial Sequence
  Description of Artificial Sequence: Synthetic peptide
AASTLQS

SEQ ID NO: 38 - protein, 7 aa, Artificial Sequence
  Description of Artificial Sequence: Synthetic peptide
WASTRES

SEQ ID NO: 39 - protein, 7 aa, Artificial Sequence
  Description of Artificial Sequence: Synthetic peptide
KASSLAN

SEQ ID NO: 40 - protein, 7 aa, Artificial Sequence
  Description of Artificial Sequence: Synthetic peptide
KASSLES

SEQ ID NO: 41 - protein, 7 aa, Artificial Sequence
  Description of Artificial Sequence: Synthetic peptide
STSNLQS

SEQ ID NO: 42 - protein, 7 aa, Artificial Sequence
  Description of Artificial Sequence: Synthetic peptide
KASRLET

SEQ ID NO: 43 - protein, 31 aa, Artificial Sequence
  Description of Artificial Sequence: Synthetic polypeptide
  MOD_RES (2)..(2): I or V
  MOD_RES (4)..(4): A, D, or S
  MOD_RES (14)..(14): E or D
  MOD_RES (24)..(24): S, P, or A
  MOD_RES (27)..(27): F or V
  MOD_RES (29)..(29): V or T
GXPXRFSGSGSGTXFTLTISSLQXEDXAXYY

SEQ ID NO: 44 - protein, 31 aa, Artificial Sequence
  Description of Artificial Sequence: Synthetic polypeptide
GIPARFSGSGSGTEFTLTISSLQSEDFAVYY

SEQ ID NO: 45 - protein, 31 aa, Artificial Sequence
  Description of Artificial Sequence: Synthetic polypeptide
GVPSRFSGSGSGTDFTLTISSLQPEDFATYY

SEQ ID NO: 46 - protein, 31 aa, Artificial Sequence
  Description of Artificial Sequence: Synthetic polypeptide
GVPDRFSGSGSGTDFTLTISSLQAEDVAVYY

SEQ ID NO: 47 - protein, 11 aa, Artificial Sequence
  Description of Artificial Sequence: Synthetic peptide
  MOD_RES (3)..(3): Q or K
  MOD_RES (4)..(4): S, A, G, Y, H
  MOD_RES (5)..(5): Y, N, F, K, G, or L
  MOD_RES (6)..(6): K, S, or R
  MOD_RES (7)..(7): T, F, Y, A, L, R, P, or S
  MOD_RES (9)..(9): Y, W, F, R, L, or I
CQXXXXXPXTF

SEQ ID NO: 48 - protein, 11 aa, Artificial Sequence [sequence omitted]

SEQ ID NO: 49 - protein, 11 aa, Artificial Sequence
  Description of Artificial Sequence: Synthetic peptide
CQQAYSFPWTF

SEQ ID NO: 50 - protein, 11 aa, Artificial Sequence [sequence omitted]

SEQ ID NO: 51 - protein, 11 aa, Artificial Sequence [sequence omitted]

SEQ ID NO: 52 - protein, 11 aa, Artificial Sequence [sequence omitted]

SEQ ID NO: 53 - protein, 11 aa, Artificial Sequence [sequence omitted]

SEQ ID NO: 54 - protein, 11 aa, Artificial Sequence [sequence omitted]

SEQ ID NO: 55 - protein, 11 aa, Artificial Sequence
  Description of Artificial Sequence: Synthetic peptide
CQQAKSLPLTF

SEQ ID NO: 56 - protein, 11 aa, Artificial Sequence
  Description of Artificial Sequence: Synthetic peptide
CQQYKSRPLTF

SEQ ID NO: 57 - protein, 11 aa, Artificial Sequence
  Description of Artificial Sequence: Synthetic peptide
CQQHGSPPFTF

SEQ ID NO: 58 - protein, 11 aa, Artificial Sequence [sequence omitted]

SEQ ID NO: 59 - protein, 11 aa, Artificial Sequence
  Description of Artificial Sequence: Synthetic peptide
CQQYLRSPITF

SEQ ID NO: 60 - protein, 9 aa, Artificial Sequence
  Description of Artificial Sequence: Synthetic peptide
  MOD_RES (2)..(2): G, Q, or P
  MOD_RES (5)..(5): K or R
  MOD_RES (6)..(6): L or V
  MOD_RES (7)..(7): E or D
GXGTXXXIK

SEQ ID NO: 61 - protein, 9 aa, Artificial Sequence
  Description of Artificial Sequence: Synthetic peptide
GQGTKVEIK

SEQ ID NO: 62 - protein, 9 aa, Artificial Sequence
  Description of Artificial Sequence: Synthetic peptide
GQGTKLEIK

SEQ ID NO: 63 - protein, 9 aa, Artificial Sequence
  Description of Artificial Sequence: Synthetic peptide
GPGTKVDIK

SEQ ID NO: 64 - protein, 108 aa, Artificial Sequence
  Description of Artificial Sequence: Synthetic polypeptide
EIVMTQSPATLSVSPGERATLSCRASQTLYTNYLAWYQQKPGQAPRLLIYGASTRATGIPARFSGSGSGTEFTLTISSLQSEDFAVYYCQQSYKTPYTFGQGTKVEIK

SEQ ID NO: 65 - protein, 107 aa, Artificial Sequence
  Description of Artificial Sequence: Synthetic polypeptide
DIQMTQSPSSLSASVGDRVTITCQASQDIIDSLNWYQQKPGKAPKLLIYAAFRLRSGVPSRFSGSGSGTDFTLTISSLQPEDFATYYCQQAYSFPWTFGQGTKLEIK

SEQ ID NO: 66-69 - skipped (4 entries)

SEQ ID NO: 70 - protein, 107 aa, Artificial Sequence
  Description of Artificial Sequence: Synthetic polypeptide
DIQMTQSPSSLSASVGDRVTITCRASRGISRWLAWYQQKPGKAPKLLIYAASSLQSGVPSRFSGSGSGTDFTLTISSLQPEDFATYYCQQGYSTPFTFGQGTKLEIK

SEQ ID NO: 71 - protein, 107 aa, Artificial Sequence
  Description of Artificial Sequence: Synthetic polypeptide
DIQMTQSPSSLSASVGDRVTITCRASQDIRRYLAWYQQKPGKAPKLLIYKASRLQSGVPSRFSGSGSGTDFTLTISSLQPEDFATYYCQQYNSYPRTFGQGTKLEIK

SEQ ID NO: 72 - protein, 107 aa, Artificial Sequence
  Description of Artificial Sequence: Synthetic polypeptide
DIQMTQSPSSLSASVGDRVTITCRASRGVSKWLAWYQQKPGKAPKLLIYAASSLQSGVPSRFSGSGSGTDFTLTISSLQPEDFATYYCQQGYSTPFTFGPGTKVDIK

SEQ ID NO: 73 - protein, 107 aa, Artificial Sequence
  Description of Artificial Sequence: Synthetic polypeptide
DIQMTQSPSSLSASVGDRVTITCRASRGVSSWLAWYQQKPGKAPKLLIYAASTLQSGVPSRFSGSGSGTDFTLTISSLQPEDFATYYCQQGYSTPFTFGPGTKVDIK

SEQ ID NO: 74 - protein, 107 aa, Artificial Sequence
  Description of Artificial Sequence: Synthetic polypeptide
EIVMTQSPATLSVSPGERATLSCRASRSVGGALAWYQQKPGQAPRLLIYGASTRATGIPARFSGSGSGTEFTLTISSLQSEDFAVYYCQQYYSTPFTFGPGTKVDIK

SEQ ID NO: 75-79 - skipped (5 entries)

SEQ ID NO: 80 - protein, 113 aa, Artificial Sequence
  Description of Artificial Sequence: Synthetic polypeptide
DIVMTQSPDSLAVSLGERATINCKSSRSVLRTSKNKNFLAWYQQKPGQPPKLLIYWASTRESGVPDRFSGSGSGTDFTLTISSLQAEDVAVYYCQQYFSAPLTFGPGTKVDIK

SEQ ID NO: 81 - protein, 107 aa, Artificial Sequence
  Description of Artificial Sequence: Synthetic polypeptide
DIQMTQSPSSLSASVGDRVTITCRASQSIRRYLNWYQQKPGKAPKLLIYKASSLANGVPSRFSGSGSGTDFTLTISSLQPEDFATYYCQKYNSAPLTFGGGTKVEIK

SEQ ID NO: 82 - protein, 107 aa, Artificial Sequence
  Description of Artificial Sequence: Synthetic polypeptide
DIQMTQSPSSLSASVGDRVTITCRASQNINKNLNWYQQKPGKAPKLLIYKASSLESGVPSRFSGSGSGTDFTLTISSLQPEDFATYYCQQAKSLPLTFGGGTKVEIK

SEQ ID NO: 83 - protein, 107 aa, Artificial Sequence
  Description of Artificial Sequence: Synthetic polypeptide
DIQMTQSPSSLSASVGDRVTITCRASQNIGSRLNWYQQKPGKAPKLLIYSTSNLQSGVPSRFSGSGSGTDFTLTISSLQPEDFATYYCQQYKSRPLTFGGGTKVEIK

SEQ ID NO: 84 - protein, 107 aa, Artificial Sequence
  Description of Artificial Sequence: Synthetic polypeptide
EIVMTQSPATLSVSPGERATLSCRASRSISDYLAWYQQKPGQAPRLLIYGASTRATGIPARFSGSGSGTEFTLTISSLQSEDFAVYYCQQHGSPPFTFGGGTKVEIK

SEQ ID NO: 85 - protein, 107 aa, Artificial Sequence
  Description of Artificial Sequence: Synthetic polypeptide
DIQMTQSPSSLSASVGDRVTITCRASQNIKRYLNWYQQKPGKAPKLLIYKASRLETGVPSRFSGSGSGTDFTLTISSLQPEDFATYYCQQSYSTPLTFGQGTRLEIK

SEQ ID NO: 86 - protein, 107 aa, Artificial Sequence
  Description of Artificial Sequence: Synthetic polypeptide
EIVMTQSPATLSVSPGERATLSCRASQSVRRKLAWYQQKPGQAPRLLIYGASTRATGIPARFSGSGSGTEFTLTISSLQSEDFAVYYCQQYLRSPITFGQGTRLEIK

SEQ ID NO: 87 - protein, 121 aa, Artificial Sequence
  Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGRDGYKGYFDYWGQGTLVTVSS

SEQ ID NO: 88-109 - skipped (22 entries)

SEQ ID NO: 110 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 111 - protein, 801 aa, Homo sapiens
MERLWGLFQRAQQLSPRSSQTVYQRVEGPRKGHLEEEEEDGEEGAETLAHFCPMELRGPEPLGSRPRQPNLIPWAAAGRRAAPYLVLTALLIFTGAFLLGYVAFRGSCQACGDSVLVVSEDVNYEPDLDFHQGRLYWSDLQAMFLQFLGEGRLEDTIRQTSLRERVAGSAGMAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQFPPVASSGLPSIPAQPISADIASRLLRKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 112 - protein, 6 aa, Artificial Sequence
  Description of Artificial Sequence: Synthetic 6xHis tag
HHHHHH

SEQ ID NO: 113 - protein, 15 aa, Artificial Sequence
  Description of Artificial Sequence: Synthetic peptide
GGGGSGGGGSGGGGS

SEQ ID NO: 114 - protein, 630 aa, Homo sapiens
MAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQFPPVASSGLPSIPAQPISADIASRLLRKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 115 - protein, 774 aa, Homo sapiens
MERLWGLFQRAQQLSPRSSQTVYQRVEGPRKGHLEEEEEDGEEGAETLAHFCPMELRGPEPLGSRPRQPNLIPWAAAGRRAAPYLVLTALLIFTGAFLLGYVAFRGSCQACGDSVLVVSEDVNYEPDLDFHQGRLYWSDLQAMFLQFLGEGRLEDTIRQTSLRERVAGSAGMAALTQDIRAALSRQKLDHVWTDTHYVGLQFPDPAHPNTLHWVDEAGKVGEQLPLEDPDVYCPYSAIGNVTGELVYAHYGRPEDLQDLRARGVDPVGRLLLVRVGVISFAQKVTNAQDFGAQGVLIYPEPADFSQDPPKPSLSSQQAVYGHVHLGTGDPYTPGFPSFNQTQKLKGPVAPQEWQGSLLGSPYHLGPGPRLRLVVNNHRTSTPINNIFGCIEGRSEPDHYVVIGAQRDAWGPGAAKSAVGTAILLELVRTFSSMVSNGFRPRRSLLFISWDGGDFGSVGSTEWLEGYLSVLHLKAVVYVSLDNAVLGDDKFHAKTSPLLTSLIESVLKQVDSPNHSGQTLYEQVVFTNPSWDAEVIRPLPMDSSAYSFTAFVGVPAVEFSFMEDDQAYPFLHTKEDTYENLHKVLQGRLPAVAQAVAQLAGQLLIRLSHDRLLPLDFGRYGDVVLRHIGNLNEFSGDLKARGLTLQWVYSARGDYIRAAEKLRQEIYSSEERDERLTRMYNVRIMRVEFYFLSQYVSPADSPFRHIFMGRGDHTLGALLDHLRLLRSNSSGTPGATSSTGFQESRFRRQLALLTWTLQGAANALSGDVWNIDNNF

SEQ ID NO: 116 - DNA, 364 bp, Artificial Sequence
  Description of Artificial Sequence: Synthetic polynucleotide
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggcgggcgcgatgggtataagggctactttgactactggggccaagggaccctggtcaccgtctcctcag

SEQ ID NO: 117 - DNA, 321 bp, Artificial Sequence
  Description of Artificial Sequence: Synthetic polynucleotide
gacatccagatgacccagtctccatcctccctgtctgcatctgtaggagacagagtcaccatcacttgccgggcaagtcagaacattaataagaacttaaattggtatcagcagaaaccagggaaagcccctaagctcctgatctataaggcatccagtttggagagtggggtcccatcaaggttcagtggcagtggatctgggacagatttcactctcaccatcagcagtctgcaacctgaagattttgcaacttactactgtcaacaggcaaaaagtctgcctctcactttcggcggagggaccaaggtggagatcaaa